SEQUENCE LISTING (116 sequences)

SEQ ID NO: 1 - protein, 116 aa, synthetic construct
QVQLQESGGGLVQPGGSLRLSCAASGGLFSINTGGWYRQAPGKQRELVATITHDDRTNYAESVKGRFTLSRDNAKNTVYLQMNSLKPEDTAVYYCRLGSAAIRGYWGQGTQVTVSS

SEQ ID NO: 2 - protein, 117 aa, synthetic construct
QVQLQESGGGLVQPGGSLRLSCAASGFTFSDHTMTWVRQAPGKGLEWVSSISSGGSRIIYADSVKGRFTISRDNAKNTLYLQMNNLRPEDTAVYFCARGTRYKMSTSGPGTQVTVSS

SEQ ID NO: 3 - protein, 117 aa, synthetic construct
EVQLVESGGGVVQPGGSLRLSCAASGFTFSDHTMTWVRQAPGKGLEWVSSISSGGSRIIYADSVKGRFTISRDNAKNTLYLQMNSLRPEDTALYYCARGTRYKLSTSGQGTLVTVSS

SEQ ID NO: 4 - protein, 116 aa, synthetic construct
EVQLVESGGGVVQPGGSLRLSCAASGGLFSINTGGWYRQAPGKQRELVATITHDDRTNYAESVKGRFTISRDNAKNTVYLQMNSLRPEDTALYYCRLGSAAIRGYWGQGTLVTVSS

SEQ ID NO: 5 - protein, 108 aa, synthetic construct
EIVLTQSPATLSLSPGERATLSCGASQSVSINFLAWYQQKPGLAPRLLIYEASSRATGIPDRFSGSGSGTDFTLTISRLEPEDFAVYYCQQYGSSPYTFGQGTKLEIK

SEQ ID NO: 6 - protein, 119 aa, synthetic construct
QLQLQESGPGLVKPSETLSLTCTVSGGSISTSSYFWGWIRQPPGKGLEWIGSIYRSGSTYYNPSLKSRVTISVDTSKNQFSLKLSSVTAADTAVYYCARGITGDPGDYWGQGTLVTVSS

SEQ ID NO: 7 - protein, 108 aa, synthetic construct
EIVLTQSPATLSLSPGERATLSCGASQSVPINFLAWYQQKPGLAPRLLIYEASSRHTGIPDRFSGSGSGTDFTLTISRLEPEDFAVYYCGQYGSSPYTFGQGTKLEIK

SEQ ID NO: 8 - protein, 119 aa, synthetic construct
QLQLQESGPGLVKPSETLSLTCTVSGGSISSSSYFWGWIRQPPGKGLEWIGSIYRSGSTYYNPSLKSRVTISVDTSKNQFSLKLSSVTAADTAVYYCARGITGDPGDYWGQGTLVTVSS

SEQ ID NO: 9 - protein, 215 aa, synthetic construct
EIVLTQSPATLSLSPGERATLSCGASQSVSINFLAWYQQKPGLAPRLLIYEASSRATGIPDRFSGSGSGTDFTLTISRLEPEDFAVYYCQQYGSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 726 aa, synthetic construct
EVQLVESGGGVVQPGGSLRLSCAASGGLFSINTGGWYRQAPGKQRELVATITHDDRTNYAESVKGRFTISRDNAKNTVYLQMNSLRPEDTALYYCRLGSAAIRGYWGQGTLVTVSSGGGGSGGGGSGGGGSEVQLVESGGGVVQPGGSLRLSCAASGFTFSDHTMTWVRQAPGKGLEWVSSISSGGSRIIYADSVKGRFTISRDNAKNTLYLQMNSLRPEDTALYYCARGTRYKLSTSGQGTLVTVSSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSQLQLQESGPGLVKPSETLSLTCTVSGGSISTSSYFWGWIRQPPGKGLEWIGSIYRSGSTYYNPSLKSRVTISVDTSKNQFSLKLSSVTAADTAVYYCARGITGDPGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 11 - protein, 215 aa, synthetic construct
EIVLTQSPATLSLSPGERATLSCGASQSVPINFLAWYQQKPGLAPRLLIYEASSRHTGIPDRFSGSGSGTDFTLTISRLEPEDFAVYYCGQYGSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 726 aa, synthetic construct
EVQLVESGGGVVQPGGSLRLSCAASGGLFSINTGGWYRQAPGKQRELVATITHDDRTNYAESVKGRFTISRDNAKNTVYLQMNSLRPEDTALYYCRLGSAAIRGYWGQGTLVTVSSGGGGSGGGGSGGGGSEVQLVESGGGVVQPGGSLRLSCAASGFTFSDHTMTWVRQAPGKGLEWVSSISSGGSRIIYADSVKGRFTISRDNAKNTLYLQMNSLRPEDTALYYCARGTRYKLSTSGQGTLVTVSSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSQLQLQESGPGLVKPSETLSLTCTVSGGSISSSSYFWGWIRQPPGKGLEWIGSIYRSGSTYYNPSLKSRVTISVDTSKNQFSLKLSSVTAADTAVYYCARGITGDPGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 13 - protein, 255 aa, Homo sapiens
  DOMAIN 24..45: note = Cysteine-rich domain 1 (CRD1)
  DOMAIN 47..86: note = Cysteine-rich domain 2 (CRD2)
  DOMAIN 87..118: note = Cysteine-rich domain 3 (CRD3)
  DOMAIN 119..159: note = Cysteine-rich domain 4 (CRD4)
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 14 - protein, 10 aa, synthetic construct
ARGTRYKLST

SEQ ID NO: 15 - protein, 10 aa, synthetic construct
ARGTRYKMST

SEQ ID NO: 16 - protein, 8 aa, synthetic construct
GFTFSDHT

SEQ ID NO: 17 - protein, 8 aa, synthetic construct
ISSGGSRI

SEQ ID NO: 18 - protein, 7 aa, synthetic construct
QSVPINF

SEQ ID NO: 19 - protein, 7 aa, synthetic construct
QSVSINF

SEQ ID NO: 20 - protein, 9 aa, synthetic construct
GQYGSSPYT

SEQ ID NO: 21 - protein, 9 aa, synthetic construct
QQYGSSPYT

SEQ ID NO: 22 - protein, 10 aa, synthetic construct
GGSISSSSYF

SEQ ID NO: 23 - protein, 10 aa, synthetic construct
GGSISTSSYF

SEQ ID NO: 24 - protein, 7 aa, synthetic construct
IYRSGST

SEQ ID NO: 25 - protein, 11 aa, synthetic construct
ARGITGDPGDY

SEQ ID NO: 26 - protein, 5 aa, synthetic construct
GGGGS

SEQ ID NO: 27 - protein, 7 aa, synthetic construct
SGGSGGS

SEQ ID NO: 28 - protein, 8 aa, synthetic construct
GGGGSGGS

SEQ ID NO: 29 - protein, 9 aa, synthetic construct
GGGGSGGGS

SEQ ID NO: 30 - protein, 10 aa, synthetic construct
GGGGSGGGGS

SEQ ID NO: 31 - protein, 15 aa, synthetic construct
GGGGSGGGGSGGGGS

SEQ ID NO: 32 - protein, 18 aa, synthetic construct
GGGGSGGGGSGGGGSGGS

SEQ ID NO: 33 - protein, 20 aa, synthetic construct
GGGGSGGGGSGGGGSGGGGS

SEQ ID NO: 34 - protein, 25 aa, synthetic construct
GGGGSGGGGSGGGGSGGGGSGGGGS

SEQ ID NO: 35 - protein, 30 aa, synthetic construct
GGGGSGGGGSGGGGSGGGGSGGGGSGGGGS

SEQ ID NO: 36 - protein, 35 aa, synthetic construct
GGGGSGGGGSGGGGSGGGGSGGGGSGGGGSGGGGS

SEQ ID NO: 37 - protein, 40 aa, synthetic construct
GGGGSGGGGSGGGGSGGGGSGGGGSGGGGSGGGGSGGGGS

SEQ ID NO: 38 - protein, 15 aa, synthetic construct
EPKSCDKTHTCPPCP

SEQ ID NO: 39 - protein, 24 aa, synthetic construct
GGGGSGGGSEPKSCDKTHTCPPCP

SEQ ID NO: 40 - protein, 12 aa, synthetic construct
EPKTPKPQPAAA

SEQ ID NO: 41 - protein, 62 aa, synthetic construct
ELKTPLGDTTHTCPRCPEPKSCDTPPPCPRCPEPKSCDTPPPCPRCPEPKSCDTPPPCPRCP

SEQ ID NO: 42 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 43 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 44 - protein, 22 aa, synthetic construct
EVGSLFANVEVCPELQGIFCYR

SEQ ID NO: 45 - protein, 22 aa, synthetic construct
EVGSSVLDVEVCPELQGIFCYR

SEQ ID NO: 46 - protein, 13 aa, synthetic construct
GGGPLGLAGSGGS

SEQ ID NO: 47 - protein, 19 aa, synthetic construct
SGRSAGGGGPLGLAGSGGS

SEQ ID NO: 48 - protein, 151 aa, synthetic construct
EVGSLFANVEVCPELQGIFCYRGGGPLGLAGSGGSEVQLVESGGGVVQPGGSLRLSCAASGGLFSINTGGWYRQAPGKQRELVATITHDDRTNYAESVKGRFTISRDNAKNTVYLQMNSLRPEDTALYYCRLGSAAIRGYWGQGTLVTVSS

SEQ ID NO: 49 - protein, 151 aa, synthetic construct
EVGSSVLDVEVCPELQGIFCYRGGGPLGLAGSGGSEVQLVESGGGVVQPGGSLRLSCAASGGLFSINTGGWYRQAPGKQRELVATITHDDRTNYAESVKGRFTISRDNAKNTVYLQMNSLRPEDTALYYCRLGSAAIRGYWGQGTLVTVSS

SEQ ID NO: 50 - protein, 157 aa, synthetic construct
EVGSLFANVEVCPELQGIFCYRSGRSAGGGGPLGLAGSGGSEVQLVESGGGVVQPGGSLRLSCAASGGLFSINTGGWYRQAPGKQRELVATITHDDRTNYAESVKGRFTISRDNAKNTVYLQMNSLRPEDTALYYCRLGSAAIRGYWGQGTLVTVSS

SEQ ID NO: 51 - protein, 157 aa, synthetic construct
EVGSSVLDVEVCPELQGIFCYRSGRSAGGGGPLGLAGSGGSEVQLVESGGGVVQPGGSLRLSCAASGGLFSINTGGWYRQAPGKQRELVATITHDDRTNYAESVKGRFTISRDNAKNTVYLQMNSLRPEDTALYYCRLGSAAIRGYWGQGTLVTVSS

SEQ ID NO: 52 - protein, 761 aa, synthetic construct
EVGSLFANVEVCPELQGIFCYRGGGPLGLAGSGGSEVQLVESGGGVVQPGGSLRLSCAASGGLFSINTGGWYRQAPGKQRELVATITHDDRTNYAESVKGRFTISRDNAKNTVYLQMNSLRPEDTALYYCRLGSAAIRGYWGQGTLVTVSSGGGGSGGGGSGGGGSEVQLVESGGGVVQPGGSLRLSCAASGFTFSDHTMTWVRQAPGKGLEWVSSISSGGSRIIYADSVKGRFTISRDNAKNTLYLQMNSLRPEDTALYYCARGTRYKLSTSGQGTLVTVSSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSQLQLQESGPGLVKPSETLSLTCTVSGGSISSSSYFWGWIRQPPGKGLEWIGSIYRSGSTYYNPSLKSRVTISVDTSKNQFSLKLSSVTAADTAVYYCARGITGDPGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 53 - protein, 761 aa, synthetic construct
EVGSSVLDVEVCPELQGIFCYRGGGPLGLAGSGGSEVQLVESGGGVVQPGGSLRLSCAASGGLFSINTGGWYRQAPGKQRELVATITHDDRTNYAESVKGRFTISRDNAKNTVYLQMNSLRPEDTALYYCRLGSAAIRGYWGQGTLVTVSSGGGGSGGGGSGGGGSEVQLVESGGGVVQPGGSLRLSCAASGFTFSDHTMTWVRQAPGKGLEWVSSISSGGSRIIYADSVKGRFTISRDNAKNTLYLQMNSLRPEDTALYYCARGTRYKLSTSGQGTLVTVSSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSQLQLQESGPGLVKPSETLSLTCTVSGGSISSSSYFWGWIRQPPGKGLEWIGSIYRSGSTYYNPSLKSRVTISVDTSKNQFSLKLSSVTAADTAVYYCARGITGDPGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 54 - protein, 767 aa, synthetic construct
EVGSLFANVEVCPELQGIFCYRSGRSAGGGGPLGLAGSGGSEVQLVESGGGVVQPGGSLRLSCAASGGLFSINTGGWYRQAPGKQRELVATITHDDRTNYAESVKGRFTISRDNAKNTVYLQMNSLRPEDTALYYCRLGSAAIRGYWGQGTLVTVSSGGGGSGGGGSGGGGSEVQLVESGGGVVQPGGSLRLSCAASGFTFSDHTMTWVRQAPGKGLEWVSSISSGGSRIIYADSVKGRFTISRDNAKNTLYLQMNSLRPEDTALYYCARGTRYKLSTSGQGTLVTVSSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSQLQLQESGPGLVKPSETLSLTCTVSGGSISSSSYFWGWIRQPPGKGLEWIGSIYRSGSTYYNPSLKSRVTISVDTSKNQFSLKLSSVTAADTAVYYCARGITGDPGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 55 - protein, 767 aa, synthetic construct
EVGSSVLDVEVCPELQGIFCYRSGRSAGGGGPLGLAGSGGSEVQLVESGGGVVQPGGSLRLSCAASGGLFSINTGGWYRQAPGKQRELVATITHDDRTNYAESVKGRFTISRDNAKNTVYLQMNSLRPEDTALYYCRLGSAAIRGYWGQGTLVTVSSGGGGSGGGGSGGGGSEVQLVESGGGVVQPGGSLRLSCAASGFTFSDHTMTWVRQAPGKGLEWVSSISSGGSRIIYADSVKGRFTISRDNAKNTLYLQMNSLRPEDTALYYCARGTRYKLSTSGQGTLVTVSSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSQLQLQESGPGLVKPSETLSLTCTVSGGSISSSSYFWGWIRQPPGKGLEWIGSIYRSGSTYYNPSLKSRVTISVDTSKNQFSLKLSSVTAADTAVYYCARGITGDPGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 56 - protein, 6 aa, synthetic construct
PLGLAG

SEQ ID NO: 57 - protein, 5 aa, synthetic construct
SGRSA

SEQ ID NO: 58 - protein, 117 aa, synthetic construct
QVQLQESGGGWVQPGGSLRLSCAASGFAFRDFTMSWARQAPGERFEWISSINPSGGSQSYLPSVKGRFTISRDNAKNTMFLQMDNLTPEDTAVYFCARGTRYKMSTSGPGTQVTVSS

SEQ ID NO: 59 - protein, 117 aa, synthetic construct
QVQLQESGGGWVQPGGSLRLSCAASGFAFRDFTMSWARQAPGERFEWISSINPSGGSQSYLPSVKGRFTISRDNAKNTMFLQMDNLTPEDTAVYFCARGTRYKMSTSGSGTQVTVSS

SEQ ID NO: 60 - protein, 117 aa, synthetic construct
QVQLQESGGGLVQAGGSLRLSCAASGDTFSSYAMGWFRQAPGERLEWVASINPSGGSQSYHPSVKDRFTISRDNGKNILFLQLDKLNPEDTAVYVCARGTRYKIFASGQGTQVTVSS

SEQ ID NO: 61 - protein, 117 aa, synthetic construct
QVQLQESGGGLVQPGGSLRLSCVASGFTFANYRMSWVRQAPGKGLEWVSSIKKSGNRTTYSDSVKGRFTISRDNAKNTMFLQMDNLTPEDTAVYFCARGTRYKMSTSGPGTQVTVSS

SEQ ID NO: 62 - protein, 10 aa, synthetic construct
ARGTRYKIFA

SEQ ID NO: 63 - protein, 8 aa, synthetic construct
GTRYKMST

SEQ ID NO: 64 - protein, 8 aa, synthetic construct
GTRYKLST

SEQ ID NO: 65 - protein, 8 aa, synthetic construct
GTRYKIFA

SEQ ID NO: 66 - protein, 8 aa, synthetic construct
GFAFRDFT

SEQ ID NO: 67 - protein, 8 aa, synthetic construct
GDTFSSYA

SEQ ID NO: 68 - protein, 8 aa, synthetic construct
GFTFANYR

SEQ ID NO: 69 - protein, 5 aa, synthetic construct
DHTMT

SEQ ID NO: 70 - protein, 5 aa, synthetic construct
DFTMS

SEQ ID NO: 71 - protein, 5 aa, synthetic construct
SYAMG

SEQ ID NO: 72 - protein, 5 aa, synthetic construct
NYRMS

SEQ ID NO: 73 - protein, 7 aa, synthetic construct
GFTFSDH

SEQ ID NO: 74 - protein, 7 aa, synthetic construct
GFAFRDF

SEQ ID NO: 75 - protein, 7 aa, synthetic construct
GDTFSSY

SEQ ID NO: 76 - protein, 7 aa, synthetic construct
GFTFANY

SEQ ID NO: 77 - protein, 8 aa, synthetic construct
INPSGGSQ

SEQ ID NO: 78 - protein, 8 aa, synthetic construct
IKKSGNRT

SEQ ID NO: 79 - protein, 17 aa, synthetic construct
SISSGGSRIIYADSVKG

SEQ ID NO: 80 - protein, 17 aa, synthetic construct
SINPSGGSQSYLPSVKG

SEQ ID NO: 81 - protein, 17 aa, synthetic construct
SINPSGGSQSYHPSVKD

SEQ ID NO: 82 - protein, 17 aa, synthetic construct
SIKKSGNRTTYSDSVKG

SEQ ID NO: 83 - protein, 6 aa, synthetic construct
SSGGSR

SEQ ID NO: 84 - protein, 6 aa, synthetic construct
NPSGGS

SEQ ID NO: 85 - protein, 6 aa, synthetic construct
KKSGNR

SEQ ID NO: 86 - protein, 8 aa, synthetic construct
GGLFSINT

SEQ ID NO: 87 - protein, 5 aa, synthetic construct
INTGG

SEQ ID NO: 88 - protein, 7 aa, synthetic construct
GGLFSIN

SEQ ID NO: 89 - protein, 7 aa, synthetic construct
ITHDDRT

SEQ ID NO: 90 - protein, 16 aa, synthetic construct
TITHDDRTNYAESVKG

SEQ ID NO: 91 - protein, 5 aa, synthetic construct
THDDR

SEQ ID NO: 92 - protein, 10 aa, synthetic construct
RLGSAAIRGY

SEQ ID NO: 93 - protein, 8 aa, synthetic construct
GSAAIRGY

SEQ ID NO: 94 - protein, 9 aa, synthetic construct
CPELQGIFC

SEQ ID NO: 95 - protein, 11 aa, synthetic construct
CPELQGIFCYR

SEQ ID NO: 96 - protein, 12 aa, synthetic construct
VEVCPELQGIFC

SEQ ID NO: 97 - protein, 14 aa, synthetic construct
VEVCPELQGIFCYR

SEQ ID NO: 98 - protein, 22 aa, synthetic construct
  VARIANT 1..11: note = Each of X represents any amino acid residue
  VARIANT 21..22: note = Each of X represents any amino acid residue
XXXXXXXXXXXCPELQGIFCXX

SEQ ID NO: 99 - protein, 22 aa, synthetic construct
  VARIANT 1..11: note = Each of X represents any amino acid residue
XXXXXXXXXXXCPELQGIFCYR

SEQ ID NO: 100 - protein, 22 aa, synthetic construct
  VARIANT 1..8: note = Each of X represents any amino acid residue
  VARIANT 21..22: note = Each of X represents any amino acid residue
XXXXXXXXVEVCPELQGIFCXX

SEQ ID NO: 101 - protein, 22 aa, synthetic construct
  VARIANT 1..8: note = Each of X represents any amino acid residue
XXXXXXXXVEVCPELQGIFCYR

SEQ ID NO: 102 - protein, 22 aa, synthetic construct
  VARIANT 5..11: note = Each of X represents any amino acid residue
  VARIANT 21..22: note = Each of X represents any amino acid residue
EVGSXXXXXXXCPELQGIFCXX

SEQ ID NO: 103 - protein, 22 aa, synthetic construct
  VARIANT 5..11: note = Each of X represents any amino acid residue
EVGSXXXXXXXCPELQGIFCYR

SEQ ID NO: 104 - protein, 22 aa, synthetic construct
  VARIANT 5..8: note = Each of X represents any amino acid residue
  VARIANT 21..22: note = Each of X represents any amino acid residue
EVGSXXXXVEVCPELQGIFCXX

SEQ ID NO: 105 - protein, 22 aa, synthetic construct
  VARIANT 5..8: note = Each of X represents any amino acid residue
EVGSXXXXVEVCPELQGIFCYR

SEQ ID NO: 106 - protein, 12 aa, synthetic construct
GASQSVPINFLA

SEQ ID NO: 107 - protein, 12 aa, synthetic construct
GASQSVSINFLA

SEQ ID NO: 108 - protein, 7 aa, synthetic construct
EASSRHT

SEQ ID NO: 109 - protein, 7 aa, synthetic construct
EASSRAT

SEQ ID NO: 110 - protein, 7 aa, synthetic construct
SSSYFWG

SEQ ID NO: 111 - protein, 7 aa, synthetic construct
TSSYFWG

SEQ ID NO: 112 - protein, 16 aa, synthetic construct
SIYRSGSTYYNPSLKS

SEQ ID NO: 113 - protein, 9 aa, synthetic construct
GITGDPGDY

SEQ ID NO: 114 - protein, 9 aa, synthetic construct
GGSISSSSY

SEQ ID NO: 115 - protein, 9 aa, synthetic construct
GGSISTSSY

SEQ ID NO: 116 - protein, 5 aa, synthetic construct
YRSGS

(19) United States
(12) Patent Application Publication
(10) Pub. No.: US 2024/0262923 A1
(43) Pub. Date: Aug. 8, 2024

(54) ANTI-PD-1×4-1BB BINDING PROTEINS

(71) Applicant: Genzyme Corporation, Cambridge, MA (US)

(72) Inventors: Marta AMARAL, FRANKFURT AM MAIN (DE); Christian BEIL, FRANKFURT AM MAIN (DE); Laurence BERTHOU-SOULIÉ, PARIS (FR); Joerg BIRKENFELD, FRANKFURT AM MAIN (DE); Béatrice CAMERON, PARIS (FR); Margot CUCCHETTI, PARIS (FR); Tarik DABDOUBI, PARIS (FR); Klervi DESRUMEAUX, PARIS (FR); Fangyong DU, SAN DIEGO, CA (US); Norbert FURTMANN, FRANKFURT AM MAIN (DE); Soraya HOELPER, FRANKFURT AM MAIN (DE); Yan LI, SAN DIEGO, CA (US); Guizhong LIU, SAN DIEGO, CA (US); Peter Peizhi LUO, SAN DIEGO, CA (US); Ercole RAO, FRANKFURT AM MAIN (DE); Ingrid SASSOON, PARIS (FR); Marion SCHNEIDER, FRANKFURT AM MAIN (DE); Fabienne SOUBRIER, PARIS (FR); Emmanuelle VIGNE, PARIS (FR)

(21) Appl. No.: 18/389,951

(22) Filed: Dec. 20, 2023

Related U.S. Application Data
(60) Provisional application No. 63/476,584, filed on Dec. 21, 2022.

(30) Foreign Application Priority Data
Apr. 13, 2023 (WO) PCT/CN2023/088095

(51) Int. Cl. C07K 16/28; C07K 14/47

(52) U.S. Cl. CPC C07K 16/2878; C07K 14/47; C07K 16/2818; C07K 2317/31; C07K 2317/35; C07K 2317/55; C07K 2317/569; C07K 2317/75; C07K 2317/76; C07K 2317/92; C07K 2319/50

(57) ABSTRACT

Anti-4-1BB and anti-PD-1 binding proteins, as well as bispecific anti-4-1BB/anti-PD-1 binding proteins, including conditionally-active derivatives thereof, are provided. Therapeutic and diagnostic methods of using binding proteins are provided.

### CROSS-REFERENCE TO RELATED APPLICATION

This application claims the benefit of priority to U.S. Provisional Application No. 63/476,584, filed Dec. 21, 2022, and claims the benefit of priority to International Application No. PCT/CN2023/088095, filed Apr. 13, 2023, both of which is herein incorporated by reference in its entirety.

### REFERENCE TO ELECTRONIC SEQUENCE LISTING

The application contains a Sequence Listing which has been submitted electronically in .XML format and is hereby incorporated by reference in its entirety. Said .XML copy, created on Dec. 19, 2023, is named “01183-0320-00US_SequenceListing.xml” and is 115,625 bytes in size. The sequence listing contained in this .XML file is part of the specification and is hereby incorporated by reference herein in its entirety.

### FIELD OF THE INVENTION

The subject disclosure relates to immunoglobulin single variable domains (ISV) against the co-stimulatory molecule 4-1BB. Via binding to 4-1BB, these proteins can activate 4-1BB-expressing cells, like T-cells. It also relates to anti-PD-1 binding proteins capable for preventing T-cell activation inhibition. It further relates to bispecific antigen-binding proteins, binding to both 4-1BB and PD-1, and to conditionally-active derivatives thereof. Such proteins are useful in the field of immuno-oncology.

### BACKGROUND

A decisive factor in the treatment of cancer is the patient's own immune system. The immune system has the capacity to attack and destroy tumor cells. However, several factors can prevent an effective reaction of the immune system, e.g., a tumor microenvironment which inhibits immune cells. In particular, tumors can prevent or interfere with the activation of T-cells.

T-cell activation is a complex process which depends on several signals. This includes the stimulation of a T-cell via T-cell receptor (TCR) signaling as well as co-stimulatory signals via other receptors. Co-stimulation is crucial for effective T-cell activation. One important co-stimulatory molecule is the tumor necrosis factor receptor superfamily member 9 (TNFRSF9, also known as 4-1BB or CD137, and encoded by the TNFRSF9 gene), a fellow of the tumor necrosis factor (TNF) receptor superfamily. Its expression is transiently induced by TCR signaling. 4-1BB was originally identified in mice by a modified differential screening procedure. The human homologue of 4-1BB was cloned from activated human T-cell leukemia virus type 1-transformed human T-lymphocytes library.

4-1BB is a co-stimulatory molecule with roles in expansion, acquisition of effector function, survival, and development of T-cell memory. In addition to being expressed on activated T-cells, NK cells, NK T-cells and T_{reg} cells, 4-1BB is also expressed on several cell types in the hematopoietic lineage, as well as endothelial and epithelial cells. 4-1BBL (TNFSF9), the primary ligand of 4-1BB, is expressed predominantly on professional antigen-presenting cell (APC) populations, B-cells, macrophages and other cell types, and binds with 4-1BB to induce signaling through TRAF1 and TRAF2 to activate the NF-κB, AKT, p38 MAPK, and ERK pathways. These signaling pathways induce the expression of survival genes encoding survivin, Bcl-2, Bcl-XL, and Bfl-1 and decrease the expression of pro-apoptotic Bim. Therefore, the 4-1BB/4-1BBL signaling pathway can promote different cell types survival. Interestingly, the phenotype in response to virus challenge differs between 4-1BB and its ligand. For instance, 4-1BBL-deficient mice show reduced accumulation of memory CD8⁺ T-cells, whereas 4-1BB-deficient mice have decreased memory but enhanced accumulation of acute CD8⁺ T-cells. This type of results indicates a bidirectional signaling or a distinct receptor signaling mechanism. Other models of infectious disease have shown that the effect of 4-1BB blockade depends upon the context and disease features. For instance, infections that are quickly cleared or produce minimal inflammation are often unaffected by 4-1BB deficiency, whereas chronic or highly inflammatory disease requires 4-1BB for memory development and/or viral clearance.

In tumor immunotherapy, it is desirable to activate the 4-1BB signaling pathway resulting in T-cell proliferation, increased effector activity (including cytokine production), memory formation, resistance to apoptosis and methylation reprogramming. An obvious approach for such a stimulation is an agonist antibody. Several such antibodies have been developed by various companies, including urelumab and utomilumab. However, this approach faces some challenges due to the structure of 4-1BB: activated 4-1BB, as well as its ligand, are mainly present as trimers on the cell surface. This can complicate effective targeting of 4-1BB with conventional antibodies, which only have two binding sites; hence can fail to provide an agonistic stimulation resulting in signal activation as effective as the 4-1BBL trimer.

In principle, this lack of stoichiometry could be compensated for by crosslinking multiple antibody molecules, e.g., by binding to Fc receptors (FcRs). However, such an approach could cause severe side effect by multiplying systemic toxic effects of 4-1BB.

Therefore, it would be advantageous to have a molecule which acts as an effective agonist for 4-1BB, but does not require any crosslinking, e.g., FcR-mediated crosslinking or any other target-mediated crosslinking such as tumor-associated antigen-mediated crosslinking, immune cell surface marker-mediated crosslinking, stromal antigen protein-mediated crosslinking or crosslinking mediated by any other target expressed in cis or trans by tumor cells, immune cells, and/or normal cells.

The present invention provides such a molecule. The activating effect of this molecule is based on an anti-4-1BB V_{HH} with pure agonist activity which activity is independent of any crosslinking.

In a further aspect, it was observed that the effect of an anti-4-1BB binding moiety could be markedly enhanced, when the anti-4-1BB binding moiety was coupled to another moiety, such as one binding to the inhibitory receptor programmed death-1 (PD-1). Hypothetically, this could be explained by a delivery of the anti-4-1BB binding moiety in cis to PD-1, whereby the anti-PD-1 binding moiety could help anchoring, or promote clusterization of, the anti-4-1BB binding moiety onto PD-1+CD8⁺ T-cells.

PD-1 (also called CD279) is a 288-amino acid protein receptor expressed on activated T-cells and B-cells, natural killer cells and monocytes. PD-1 is a member of the CD28/CTLA-4 (cytotoxic T lymphocyte antigen)/ICOS (inducible co-stimulator) family of T-cell co-inhibitory receptors. Its primary function is to attenuate the immune response. PD-1 has two ligands, PD-Ligand 1 (PD-L1) and PD-L2. PD-L1 (also called CD274 or B7H1) is expressed widely on both lymphoid and non-lymphoid tissues such as CD4⁺ and CD8⁺ T-cells, macrophage lineage cells, peripheral tissues as well as on tumor cells and virally-infected cells. PD-L2 (also called CD273 or B7-DC) has a more restricted expression than PD-L1, being expressed on activated dendritic cells and macrophages. PD-L1 is expressed in most human cancers, including melanoma, glioma, non-small cell lung cancer, squamous cell carcinoma of head and neck, leukemia, pancreatic cancer, renal cell carcinoma, and hepatocellular carcinoma, and may be inducible in nearly all cancer types. PD-1 binding to its ligands results in decreased T-cell proliferation and cytokine secretion, compromising humoral and cellular immune responses in diseases such as cancer or viral infection. Blockade of PD-1 binding to reverse immunosuppression has been studied in viral and tumor immunotherapy.

T-cell co-stimulatory and co-inhibitory molecules (collectively named co-signaling molecules) play a crucial role in regulating T-cell activation, subset differentiation, effector function and survival. Following recognition of cognate peptide-MHC complexes on APC by the TCR, co-signaling receptors co-localize with TCR at the immune synapse, where they synergize with TCR signaling to promote or inhibit T-cell activation and function. The ultimate immune response is regulated by a balance between co-stimulatory and co-inhibitory signals (“immune checkpoints”). PD-1 functions as one such “immune checkpoint” in mediating peripheral T-cell tolerance and in avoiding autoimmunity: PD-1 binding to PD-L1 or PD-L2 inhibits T-cell activation. This ability of PD-1 to inhibit T-cell activation is exploited by chronic viral infections and tumors to evade immune response. In chronic viral infections, PD-1 is highly expressed on virus-specific T-cells and these T-cells become “exhausted” with loss of effector functions and proliferative capacity. PD-L1 is expressed on a wide variety of tumors and studies on animal models have shown that PD-L1 on tumors inhibits T-cell activation and lysis of tumor cells and may lead to increased death of tumor-specific T-cells. The PD-1/PD-L1 complex also plays an important role in induced T-regulatory (T_{reg}) cell development and in sustaining T_{reg} function.

Since PD-1 plays an important role in autoimmunity, tumor immunity and infectious immunity, it is an ideal target for immunotherapy. Blocking PD-1 with antagonists, including monoclonal antibodies, has been studied in treatments of cancer and chronic viral infections.

The present invention provides such PD-1 antagonists.

### BRIEF SUMMARY OF THE INVENTION

In a first aspect, a multispecific antigen-binding protein comprising at least one immunoglobulin single variable domain (ISV) specifically binding to 4-1BB is provided, wherein the at least one ISV has pure agonist activity.

In some embodiments, a pure agonist activity means that the at least one ISV is capable of activating a T-cell via 4-1BB signaling (i) in soluble conditions, and/or (ii) in the absence of a cross-linking reagent, and/or (iii) in an FcγR-independent manner (i.e., independently of Fcγ receptor engagement), and/or (iv) in the absence of target-mediated crosslinking of 4-1BB. In some embodiments, the pure agonist activity may be determined by a NF-κB pathway activation assay in the absence of a cross-linking reagent, such as, e.g., in absence of anti-human Fab antibody.

In some embodiments, the at least one ISV competes with 4-1BBL for 4-1BB binding.

In some embodiments, the at least one ISV interacts with the cysteine-rich domain 2 (CRD2) and/or cysteine-rich domain 3 (CRD3) domain of 4-1BB; preferably the ISV interacts with the CRD2 and CRD3 domains of 4-1BB.

In some embodiment, 4-1BB is human 4-1BB, an exemplary amino acid sequence of which is SEQ ID NO: 13.

In some embodiments, the at least one ISV interacts with at least one amino acid residue of 4-1BB selected from the group consisting of residues K69, G70, V71, F72, R73, F92, L95, S100, M101, C102, E103, Q104, K114, K115, and G116 of SEQ TD NO: 13.

In some embodiments, the at least one ISV is a V_{HH}.

In some embodiments, the at least one ISV comprises three complementary determining regions CDR1, CDR2 and CDR3; and CDR3 comprises or consists of the amino acid sequence ARGTRYKLST (SEQ ID NO: 14), ARGTRYKMST (SEQ ID NO: 15), or ARGTRYKIFA (SEQ ID NO: 62).

In some embodiments, CDR1 comprises or consists of the amino acid sequence GFTFSDHT (SEQ ID NO: 16), GFAFRDFT (SEQ ID NO: 66), GDTFSSYA (SEQ ID NO: 67), or GFTFANYR (SEQ ID NO: 68).

In some embodiments, CDR2 comprises or consists of the amino acid sequence ISSGGSRI (SEQ ID NO: 17), INPSGGSQ (SEQ ID NO: 77), or IKKSGNRT (SEQ ID NO: 78).

In some embodiments, the at least one ISV comprises or consists of:
(i) an amino acid sequence selected from the group consisting of SEQ ID NOs: 2, 3, 58, 59, 60 and 61, or
(ii) an amino acid sequence sharing at least 70% of sequence identity over the non-CDR regions of SEQ ID NOs: 2, 3, 58, 59, 60 or 61.

In some embodiments, the ISV comprises or consists of an amino acid sequence of SEQ ID NO: 2 or 3. In some embodiments, the ISV comprises or consists of an amino acid sequence of SEQ ID NO: 3.

In some embodiments, the multispecific antigen-binding protein comprises at least two ISVs specifically binding to 4-1BB.

In some embodiments, the at least two ISVs specifically binding to 4-1BB are identical. Alternatively, the at least two ISVs specifically binding to 4-1BB may be different and may then bind (i) to the same epitope, (ii) to overlapping epitopes, or (iii) to distinct epitopes of 4-1BB. In some embodiments, the at least two ISVs specifically binding to 4-1BB are different and bind to distinct epitopes of 4-1BB.

In some embodiments, at least a second ISV specifically binding to 4-1BB comprises or consists of:
an amino acid sequence selected from the group consisting of SEQ ID NOs: 1 and 4, or
an amino acid sequence sharing at least 70% sequence identity over the non-CDR regions of SEQ ID NOs: 1 or 4.

In some embodiments, the at least second ISV comprises or consists of an amino acid sequence with SEQ ID NO: 4.

In some embodiments, the multispecific antigen-binding protein comprises at least two ISVs, wherein one of the at least two ISVs specifically binds to 4-1BB, and the other one of the at least two ISVs specifically binds to another target antigen. In some embodiments, the other target antigen may be a T-cell antigen, a tumor-associated antigen (TAA) or tumor-specific antigen (TSA), or a non-self antigen.

In some embodiments, the multispecific antigen-binding protein comprises at least four ISVs specifically binding to 4-1BB. In some embodiments, these at least four ISVs comprise:
(i) a first set of at least two identical ISVs specifically binding to 4-1BB, and (ii) a second set of at least two other identical ISVs specifically binding to 4-1BB; or
(i′) a first set of at least two ISVs specifically binding to a first epitope of 4-1BB, and (ii′) a second set of at least two other ISVs specifically binding to a second epitope of 4-1BB.

In some embodiments, the first set of at least two ISVs of (i) or (i′) above are ISVs specifically binding to 4-1BB with pure agonist activity as defined above (for instance, but not limited to, an ISV comprising or consisting of an amino acid sequence of SEQ ID NO: 2 or 3).

In some embodiments, the second set of at least two ISVs of (ii) or (ii′) above are ISVs specifically binding to 4-1BB as defined above (for instance, but not limited to, an ISV comprising or consisting of an amino acid sequence of SEQ ID NOs: 1 or 4).

In some embodiments, the multispecific antigen-binding protein further comprises an antibody Fc region or a fragment thereof. In some embodiments, the Fc region or fragment thereof is ADCC- and/or ADCP-silenced.

In some embodiments, the multispecific antigen-binding protein further comprises at least one Fab fragment.

In some embodiments, the multispecific antigen-binding protein comprises:
(a) a first polypeptide comprising, preferably from N-terminus to C-terminus:
   a first ISV specifically binding to 4-1BB;
   a second ISV specifically binding to 4-1BB, preferably wherein the second ISV is different from the first ISV;
   at least one C_{H} domain of a Fc region; and
   a variable and constant domain of a Fab fragment;
(b) a second polypeptide comprising a variable and constant domain of a Fab fragment; wherein the variable and constant domains of the first and second polypeptide form a Fab fragment.

In some embodiments, the multispecific antigen-binding protein further comprises a third and fourth polypeptide identical to the first and second polypeptide, respectively, wherein the at least one C_{H} domain of the first and third polypeptides form an Fc region.

In some embodiments,
the variable and constant domain of the first polypeptide are a V_{H} and C_{H}1 domain, and the variable and constant domain of the second polypeptide are a V_{L} and C_{L} domain; or
the variable and constant domain of the first polypeptide are a V_{L} and C_{L} domain, and the variable and constant domain of the second polypeptide are a V_{H} and C_{H}1 domain.

In some embodiments, the at least one C_{H} domain of the first polypeptide comprise:
C_{H}2 and C_{H}3 domains of IgG;
C_{H}2 and C_{H}3 domains of IgD;
C_{H}2 and C_{H}3 domains of IgA;
C_{H}2, C_{H}3 and C_{H}4 domains of IgM; or
C_{H}2, C_{H}3, and C_{H}4 domains of IgE.

In some embodiments, the at least one C_{H} domain of the first polypeptide comprise C_{H}2 and C_{H}3 domains of IgG. In some embodiments, the at least one C_{H} domain of the first polypeptide comprise C_{H}2 and C_{H}3 domains of IgG1 or IgG4. In some embodiments, the at least one C_{H} domain of the first polypeptide comprise C_{H}2 and C_{H}3 domains of IgG1.

In some embodiments, the first polypeptide of the multispecific antigen-binding protein comprises, preferably from N-terminus to C-terminus:
a first ISV specifically binding to 4-1BB;
a first linker;
a second ISV specifically binding to 4-1BB, preferably wherein the second ISV is different from the first ISV;
a second linker;
an IgG hinge region;
an IgG C_{H}2 domain;
and IgG C_{H}3 domain;
a third linker;
a V_{H} domain of a Fab fragment; and
a C_{H}1 domain of a Fab fragment.

In some embodiments, the second polypeptide of the multispecific antigen-binding protein comprises, preferably from N-terminus to C-terminus:
a V_{L} domain of a Fab fragment; and
a C_{L} domain of a Fab fragment.

In some embodiments, the at least one Fab fragment binds specifically to a B- and/or T-cell surface protein other than 4-1BB. In some embodiments, the at least one Fab fragment binds specifically to an immune checkpoint molecule. In some embodiments, the at least one Fab fragment is a PD-1 antagonist. In some embodiments, the at least one Fab fragment is an antigen-binding protein that specifically binds to PD-1, and which comprises:
three light chain complementarity determining region (CDR) sequences found in SEQ ID NO: 7 or 5, and
three heavy chain CDR sequences found in SEQ ID NO: 8 or 6.

In some embodiments, the at least one Fab fragment is an antigen-binding protein that specifically binds to PD-1, and which comprises:
(i) a light chain variable region comprising the three following CDR sequences:
   VL-CDR1: QSVPINF (SEQ ID NO: 18) or QSVSINF (SEQ ID NO: 19);
   VL-CDR2: EAS; and
   VL-CDR3: GQYGSSPYT (SEQ ID NO: 20) or QQYGSSPYT (SEQ ID NO: 21); and
(ii) a heavy chain variable region comprising the three following CDR sequences:
   VH-CDR1: GGSISSSSYF (SEQ ID NO: 22) or GGSISTSSYF (SEQ ID NO: 23);
   VH-CDR2: IYRSGST (SEQ ID NO: 24); and
   VH-CDR3: ARGITGDPGDY (SEQ ID NO: 25).

In some embodiments, the at least one Fab fragment is an antigen-binding protein that specifically binds to PD-1, and which comprises:
(i) a light chain variable region comprising the three following CDR sequences:
   VL-CDR1: QSVPINF (SEQ ID NO: 18);
   VL-CDR2: EAS; and
   VL-CDR3: GQYGSSPYT (SEQ ID NO: 20); and
(ii) a heavy chain variable region comprising the three following CDR sequences:
   VH-CDR1: GGSISSSSYF (SEQ ID NO: 22);
   VH-CDR2: IYRSGST (SEQ ID NO: 24); and
   VH-CDR3: ARGITGDPGDY (SEQ ID NO: 25).

In some embodiments, the at least one Fab fragment is an antigen-binding protein that specifically binds to PD-1, and which comprises:
a light chain variable region with SEQ ID NO: 7 or 5, or a light chain variable region sharing at least 70% of sequence identity over the non-CDR regions of SEQ ID NO: 7 or 5; and
a heavy chain variable region with SEQ ID NO: 8 or 6, or a heavy chain variable region sharing at least 70% of sequence identity over the non-CDR regions of SEQ ID NO: 8 or 6.

In some embodiments, the at least one Fab fragment is an antigen-binding protein that specifically binds to PD-1, and comprises a light chain variable region with SEQ ID NO: 7 and a heavy chain variable region with SEQ ID NO: 8.

In some embodiments, the multispecific antigen-binding protein provided herein comprises
at least a first polypeptide with SEQ ID NO: 11 or 9, and at least a second polypeptide with SEQ ID NO: 12 or 10; or
at least a first polypeptide sharing at least 70% of sequence identity over the non-CDR regions of SEQ ID NO: 11 or 9, and at least a second polypeptide sharing at least 70% of sequence identity over the non-CDR regions of SEQ ID NO: 12 or 10.

In some embodiments, the multispecific antigen-binding protein provided herein comprises at least a first polypeptide with SEQ ID NO: 11 and at least a second polypeptide with SEQ ID NO: 12.

In some embodiments, the multispecific antigen-binding protein provided herein comprises at least a first polypeptide with SEQ ID NO: 9 and at least a second polypeptide with SEQ ID NO: 10.

In a second aspect, a conditionally-active multispecific antigen-binding protein is provided.

In some embodiments, the conditionally-active multispecific antigen-binding protein comprises:
the multispecific antigen-binding protein described above, and
at least one masking moiety reducing or inhibiting the binding of the multispecific antigen-binding protein to at least one of its target antigens.

In some embodiments, the at least one masking moiety comprises or consists of an amino acid sequence of SEQ ID NO: 97, or an amino acid sequence sharing at least 70% of sequence identity with SEQ ID NO: 97. In some embodiments, the at least one masking moiety comprises or consists of an amino acid sequence of SEQ ID NO: 44 or 45, or an amino acid sequence sharing at least 70% of sequence identity with SEQ ID NO: 44 or 45.

In some embodiments, the conditionally-active multispecific antigen-binding protein further comprises at least one linker between the multispecific antigen-binding protein and the masking moiety. In some embodiments, the at least one linker is cleavable. In some embodiments, the at least one linker is cleavable by at least one tumor-specific protease. In some embodiments, the at least one tumor-specific protease is selected from the group consisting of matrix metalloproteinase-9 (MMP-9), urokinase-type plasminogen activator (uPa), matrix metalloproteinase-2 (MMP-2), matriptase, legumain, kallikrein-related peptidase-3, human neutrophil elastase, proteinase 3 (Pr3), cathepsin B, and cathepsin K. In some embodiments, the at least one tumor-specific protease is MMP-9 or uPa, or a combination thereof. In some embodiments, the at least one linker comprises an amino acid sequence of SEQ ID NO: 56 and/or 57. In some embodiments, the at least one linker comprises or consists of an amino acid sequence of SEQ ID NO: 46 or 47.

In some embodiments, the conditionally-active multispecific antigen-binding protein comprises:
a. at least a first polypeptide with SEQ ID NO: 11 or 9; and
b. at least a second polypeptide with SEQ ID NO: 52, 53, 54 or 55.

In some embodiments, the conditionally-active multispecific antigen-binding protein further comprises a third and fourth polypeptide identical to the first and second polypeptide, respectively.

In a third aspect, an immunoglobulin single variable domain (ISV) specifically binding to 4-1BB is provided, wherein the ISV has pure agonist activity.

In some embodiments, a pure agonist activity means that the ISV is capable of activating a T-cell via 4-1BB signaling (i) in soluble conditions, and/or (ii) in the absence of a cross-linking reagent, and/or (iii) in an FcγR-independent manner (i.e., independently of Fcγ receptor engagement), and/or (iv) in the absence of target-mediated crosslinking of 4-1BB. In some embodiments, the pure agonist activity may be determined by a NF-κB pathway activation assay in the absence of a cross-linking reagent.

In some embodiments, the ISV competes with 4-1BBL for 4-1BB binding.

In some embodiments, the ISV interacts with the cysteine-rich domain 2 (CRD2) and/or cysteine-rich domain 3 (CRD3) domain of 4-1BB. In some embodiments, the ISV interacts with the CRD2 and CRD3 domains of 4-1BB.

In some embodiment, 4-1BB is human 4-1BB, an exemplary amino acid sequence of which is SEQ ID NO: 13.

In some embodiments, the ISV interacts with one or several amino acid residues of 4-1BB selected from the group consisting of residues K69, G70, V71, F72, R73, F92, L95, S100, M101, C102, E103, Q104, K114, K115 and G116 of SEQ ID NO: 13.

In some embodiments, the ISV is a V_{HH}.

In some embodiments, the ISV comprises three complementary determining regions CDR1, CDR2 and CDR3; and CDR3 comprises or consists of the amino acid sequence ARGTRYKLST (SEQ ID NO: 14), ARGTRYKMST (SEQ ID NO: 15), or ARGTRYKIFA (SEQ ID NO: 62).

In some embodiments, CDR1 comprises or consists of the amino acid sequence GFTFSDHT (SEQ ID NO: 16), GFAFRDFT (SEQ ID NO: 66), GDTFSSYA (SEQ ID NO: 67), or GFTFANYR (SEQ ID NO: 68).

In some embodiments, CDR2 comprises or consists of the amino acid sequence ISSGGSRI (SEQ ID NO: 17), INPSGGSQ (SEQ ID NO: 77), or IKKSGNRT (SEQ ID NO: 78).

In some embodiments, the ISV comprises or consists of:
(i) an amino acid sequence selected from the group consisting of SEQ ID NOs: 2, 3, 58, 59, 60 and 61, or
(ii) an amino acid sequence sharing at least 70% of sequence identity over the non-CDR regions of SEQ ID NOs: 2, 3, 58, 59, 60 or 61.

In some embodiments, the ISV comprises or consists of an amino acid sequence of SEQ ID NO: 2 or 3. In some embodiments, the ISV comprises or consists of an amino acid sequence of SEQ ID NO: 3.

In some embodiments, the ISV is a V_{HH}.

In a fourth aspect, an immunoglobulin single variable domain (ISV) specifically binding to 4-1BB is provided, wherein the ISV comprises or consists of:
an amino acid sequence selected from the group consisting of SEQ ID NOs: 1 and 4, or
an amino acid sequence sharing at least 70% sequence identity over the non-CDR regions of SEQ ID NOs: 1 or 4.

In some embodiments, the ISV comprises or consists of an amino acid sequence with SEQ ID NO: 4.

In some embodiments, the ISV is a V_{HH}.

In a fifth aspect, a bivalent or bispecific antigen-binding protein comprising at least one immunoglobulin single variable domain (ISV) is provided, wherein the bivalent or bispecific antigen-binding protein comprises at least a first ISV specifically binding to 4-1BB with pure agonist activity as defined above (for instance, but not limited to, an ISV comprising or consisting of an amino acid sequence of SEQ ID NO: 2 or 3), and at least a second ISV specifically binding to the same or another target antigen.

In some embodiments, the other target antigen may be a T-cell antigen, a tumor-associated antigen (TAA) or tumor-specific antigen (TSA), or a non-self antigen.

In some embodiments, the at least second ISV is an ISV specifically binding to 4-1BB as defined above (for instance, but not limited to, an ISV comprising or consisting of an amino acid sequence of SEQ ID NOs: 1 or 4).

In a sixth aspect, a conditionally-active immunoglobulin single variable domain (ISV) specifically binding to 4-1BB is provided, wherein the conditionally-active ISV comprises:
an ISV specifically binding to 4-1BB as defined above (for instance, but not limited to, an ISV comprising or consisting of an amino acid sequence of SEQ ID NOs: 1 or 4), and
at least one masking moiety reducing or inhibiting the binding of the ISV to its target antigen.

In some embodiments, the at least one masking moiety comprises or consists of an amino acid sequence of SEQ ID NO: 97, or an amino acid sequence sharing at least 70% of sequence identity with SEQ ID NO: 97. In some embodiments, the at least one masking moiety comprises or consists of an amino acid sequence of SEQ ID NO: 44 or 45, or an amino acid sequence sharing at least 70% of sequence identity with SEQ ID NO: 44 or 45.

In some embodiments, the conditionally-active ISV further comprises at least one linker between the ISV and the masking moiety. In some embodiments, the at least one linker is cleavable. In some embodiments, the at least one linker is cleavable by at least one tumor-specific protease. In some embodiments, the at least one tumor-specific protease is selected from the group consisting of matrix metalloproteinase-9 (MMP-9), urokinase-type plasminogen activator (uPa), matrix metalloproteinase-2 (MMP-2), matriptase, legumain, kallikrein-related peptidase-3, human neutrophil elastase, proteinase 3 (Pr3), cathepsin B, and cathepsin K. In some embodiments, the at least one tumor-specific protease is MMP-9 or uPa, or a combination thereof. In some embodiments, the at least one linker comprises an amino acid sequence of SEQ ID NO: 56 and/or 57. In some embodiments, the at least one linker comprises or consists of an amino acid sequence of SEQ ID NO: 46 or 47.

In some embodiments, the ISV is a V_{HH}.

In some embodiments, the conditionally-active ISV comprises or consists of an amino acid sequence selected from the group consisting of SEQ ID NOs: 48, 49, 50 and 51.

In a seventh aspect, a conditionally-active immunoglobulin single variable domain (ISV) specifically binding to 4-1BB is provided, wherein the conditionally-active ISV comprises:
an ISV specifically binding to 4-1BB; and
at least one masking moiety reducing or inhibiting the binding of the ISV to 4-1BB.

In some embodiments, the at least one masking moiety comprises or consists of an amino acid sequence of SEQ ID NO: 97, or an amino acid sequence sharing at least 70% of sequence identity with SEQ ID NO: 97. In some embodiments, the at least one masking moiety comprises or consists of an amino acid sequence of SEQ ID NO: 44 or 45, or an amino acid sequence sharing at least 70% of sequence identity with SEQ ID NO: 44 or 45.

In some embodiments, the conditionally-active ISV further comprises at least one linker between the ISV and the masking moiety. In some embodiments, the at least one linker is cleavable. In some embodiments, the at least one linker is cleavable by at least one tumor-specific protease. In some embodiments, the at least one tumor-specific protease is selected from the group consisting of matrix metalloproteinase-9 (MMP-9), urokinase-type plasminogen activator (uPa), matrix metalloproteinase-2 (MMP-2), matriptase, legumain, kallikrein-related peptidase-3, human neutrophil elastase, proteinase 3 (Pr3), cathepsin B, and cathepsin K. In some embodiments, the at least one tumor-specific protease is MMP-9 or uPa, or a combination thereof. In some embodiments, the at least one linker comprises an amino acid sequence of SEQ ID NO: 56 and/or 57. In some embodiments, the at least one linker comprises or consists of an amino acid sequence of SEQ ID NO: 46 or 47.

In some embodiments, the ISV is a V_{HH}.

In an eighth aspect, a conditionally-active immunoglobulin single variable domain (ISV) specifically binding to a target antigen is provided, wherein the conditionally-active ISV comprises:
an ISV specifically binding to a target antigen; and
at least one masking moiety reducing or inhibiting the binding of the ISV to its target antigen.

In some embodiments, the at least one masking moiety comprises or consists of an amino acid sequence of SEQ ID NO: 97, or an amino acid sequence sharing at least 70% of sequence identity with SEQ ID NO: 97. In some embodiments, the at least one masking moiety comprises or consists of an amino acid sequence of SEQ ID NO: 44 or 45, or an amino acid sequence sharing at least 70% of sequence identity with SEQ ID NO: 44 or 45.

In some embodiments, the conditionally-active ISV further comprises at least one linker between the ISV and the masking moiety. In some embodiments, the at least one linker is cleavable. In some embodiments, the at least one linker is cleavable by at least one tumor-specific protease. In some embodiments, the at least one tumor-specific protease is selected from the group consisting of matrix metalloproteinase-9 (MMP-9), urokinase-type plasminogen activator (uPa), matrix metalloproteinase-2 (MMP-2), matriptase, legumain, kallikrein-related peptidase-3, human neutrophil elastase, proteinase 3 (Pr3), cathepsin B, and cathepsin K. In some embodiments, the at least one tumor-specific protease is MMP-9 or uPa, or a combination thereof. In some embodiments, the at least one linker comprises an amino acid sequence of SEQ ID NO: 56 and/or 57. In some embodiments, the at least one linker comprises or consists of an amino acid sequence of SEQ ID NO: 46 or 47.

In some embodiments, the ISV is a V_{HH}.

In a ninth aspect, an antibody or antigen-binding fragment thereof is provided, wherein the antibody or antigen-binding fragment thereof comprises
(i) three light chain complementarity determining region (CDR) sequences set forth in SEQ ID NOs: 5 or 7, and
(ii) three heavy chain CDR sequences set forth in SEQ ID NOs: 6 or 8.

In some embodiments, the antibody or antigen-binding fragment thereof specifically binds to PD-1. In some embodiments, PD-1 is human PD-1, an exemplary amino sequence of which is SEQ ID NO: 42.

In some embodiments, the antibody or antigen-binding fragment thereof comprises:
(i) a light chain variable region comprising the three following CDR sequences:
   a. V_{L}-CDR1: QSVPINF (SEQ ID NO: 18) or QSVSINF (SEQ ID NO: 19);
   b. V_{L}-CDR2: EAS; and
   c. V_{L}-CDR3: GQYGSSPYT (SEQ ID NO: 20) or QQYGSSPYT (SEQ ID NO: 21); and
(ii) a heavy chain variable region comprising the three following CDR sequences:
   a. V_{H}-CDR1: GGSISSSSYF (SEQ ID NO: 22) or GGSISTSSYF (SEQ ID NO: 23);
   b. V_{H}-CDR2: IYRSGST (SEQ ID NO: 24); and
   c. V_{H}-CDR3: ARGITGDPGDY (SEQ ID NO: 25).

In some embodiments, the antibody or antigen-binding fragment thereof comprises:
(i) a light chain variable region comprising the three following CDR sequences:
   a. V_{L}-CDR1: QSVPINF (SEQ ID NO: 18);
   b. V_{L}-CDR2: EAS; and
   c. V_{L}-CDR3: GQYGSSPYT (SEQ ID NO: 20); and
(ii) a heavy chain variable region comprising the three following CDR sequences:
   a. V_{H}-CDR1: GGSISSSSYF (SEQ ID NO: 22);
   b. V_{H}-CDR2: IYRSGST (SEQ ID NO: 24); and
   c. V_{H}-CDR3: ARGITGDPGDY (SEQ ID NO: 25).

In some embodiments, the antibody or antigen-binding fragment thereof comprises:
(i) a light chain variable region with SEQ ID NO: 7 or 5, or a light chain variable region sharing at least 70% of sequence identity over the non-CDR regions of SEQ ID NO: 7 or 5; and
(ii) a heavy chain variable region with SEQ ID NO: 8 or 6, or a heavy chain variable region sharing at least 70% of sequence identity over the non-CDR regions of SEQ ID NO: 8 or 6.

In some embodiments, the antibody or antigen-binding fragment thereof comprises alight chain variable region with SEQ ID NO: 7 and a heavy chain variable region with SEQ ID NO: 8.

In some embodiments, the antibody or antigen-binding fragment thereof comprises alight chain variable region with SEQ ID NO: 5 and a heavy chain variable region with SEQ ID NO: 6.

In some other aspects, provided are compositions, comprising the multispecific antigen-binding protein disclosed herein; or the conditionally-active multispecific antigen-binding protein disclosed herein; or the immunoglobulin single variable domains (ISV) disclosed herein; or the bivalent or bispecific antigen-binding protein disclosed herein; or the conditionally-active immunoglobulin single variable domain (ISV) disclosed herein; or the antibody or antigen-binding fragment thereof disclosed herein; and a pharmaceutically acceptable carrier or excipient.

In some other aspects, provided are methods of treating a subject in need thereof, comprising administering an effective amount of one of the compositions disclosed herein. In some embodiments, the subject has cancer. In some embodiments, the subject is a human.

In some other aspects, provided are one of the compositions disclosed herein for use in treating cancer in a subject in need thereof. In some embodiments, the subject is a human.

Also provided herein are isolated polynucleotides encoding any of the multispecific antigen-binding protein disclosed herein; or the conditionally-active multispecific antigen-binding protein disclosed herein; or the immunoglobulin single variable domains (ISV) disclosed herein; or the bivalent or bispecific antigen-binding protein disclosed herein; or the conditionally-active immunoglobulin single variable domain (ISV) disclosed herein; or the antibody or antigen-binding fragment thereof disclosed herein. Also provided herein are vectors comprising such polynucleotide; as well as host cells comprising such polynucleotide.

Also provided herein are methods of making any of the multispecific antigen-binding protein disclosed herein; or the conditionally-active multispecific antigen-binding protein disclosed herein; or the immunoglobulin single variable domains (ISV) disclosed herein; or the bivalent or bispecific antigen-binding protein disclosed herein; or the conditionally-active immunoglobulin single variable domain (ISV) disclosed herein; or the antibody or antigen-binding fragment thereof disclosed herein; which methods comprise expressing one of the polynucleotides above in a cell.

The summary of the disclosure described above is non-limiting and other features and advantages of the disclosed antigen-binding proteins and methods will be apparent from the following brief description of the drawings, detailed description of the disclosure, and claims.

### BRIEF DESCRIPTION OF THE FIGURES

FIG. 1 schematically depicts a “Fab-like” construct as used in Example 1. In each Fab-like construct, one V_{HH} is fused to a human C_{L} domain and another one is fused to a human IgG1 C_{H}1 domain: the two V_{HH}s associate through C_{H}1-C_{L} interaction. Human influenza hemagglutinin [HA] and 6-His [His] tags are used for purification purposes. These Fab-like constructs can be monospecific: either bivalent monoparatopic (i.e., comprising twice the same V_{HH} against the same target), or bivalent biparatopic (i.e., two different V_{HH}s against the same target); or they can be bispecific (i.e., one V_{HH} against a first target and one V_{HH} against another target). They can also be monovalent (i.e., one V_{HH} against a first target and one V_{HH} directed to an irrelevant target).

FIG. 2 is a graph depicting cross-reactivity of seven anti-4-1BB V_{HH} clones (clones #1 to 7) with human 4-1BB [h4-1BB], cynomolgus monkey 4-1BB [c4-1BB], and two other members of the tumor necrosis factor receptor superfamily/TNFRSF (human OX40 [hOX40] and human CD40 [CD40]).

FIG. 3 depicts the results of a competition ELISA assay between three anti-4-1BB V_{HH}s (clones #2, 3 and 5) and 4-1BB natural ligand, 4-1BBL.

FIGS. 4A-4F depict the activation of the NF-κB pathway via 4-1BB in reporter functional assays, shown as signal-to-background ratio (S/B). The graphs show the activation of the NF-κB pathway in genetically engineered Jurkat T-cells that constitutively express 4-1BB using increasing doses of anti-4-1BB V_{HH} clones:
in a bivalent monoparatopic Fab-like format (i.e., with two identical anti-4-1BB V_{HH}s) in presence of a cross-linker reagent (anti-Fab antibody): FIG. 4A;
in a bivalent monoparatopic Fab-like format (i.e., with two identical anti-4-1BB V_{HH}s) in absence of cross-linker reagent: FIG. 4B;
in a monovalent Fab-like format (i.e., with one anti-4-1BB V_{HH} and one V_{HH} to an irrelevant target, e.g., FMDV) in presence of a cross-linker reagent (anti-Fab antibody): FIG. 4C;
in a monovalent Fab-like format (i.e., with one anti-4-1BB V_{HH} and one V_{HH} to an irrelevant target, e.g., FMDV) in absence of cross-linker reagent: FIG. 4D;
in a bivalent monoparatopic Fab-like format (i.e., with two identical anti-4-1BB V_{HH}s) versus bivalent biparatopic Fab-like format (i.e., with two different anti-4-1BB V_{HH}s) in absence of cross-linker reagent: FIG. 4E; and
in a bispecific Fab-like format (i.e., with one anti-4-1BB V_{HH} and one V_{HH} to another target, e.g., CD28): FIG. 4F.

FIGS. 5A-5B depict the activation of the NF-κB pathway via 4-1BB in reporter functional assays, shown as signal-to-background ratio (S/B), using genetically engineered Jurkat T-cells that constitutively express 4-1BB, induced by increasing doses of anti-4-1BB V_{HH} clones in a bispecific Fab-like format (i.e., with one anti-4-1BB V_{HH} and one V_{HH} to another target, e.g., OX40), in absence (FIG. 5A) or in presence (FIG. 5B) of OX40⁺ cells.

FIG. 6 is an X-ray crystallography 3-D structure of 4-1BBL and the anti-4-1BB clone #2 and clone #5 V_{HH}s, in complex with the extracellular domain of human 4-1BB (amino acid residues 24-186 of SEQ ID NO: 13). 4-1BB is shown in “surface” representation; 4-1BBL and the two anti-4-1BB V_{HH}s are shown in “cartoon” representation. Cysteine-rich domains 1-4 of 4-1BB (CRD1-CRD4) are indicated with arrows.

FIGS. 7A-7B depict the properties of four anti-4-1BB clone #5 variants (clones #5a-5d): binding assessed by ELISA (FIG. 7A) and activation of the NF-κB pathway via 4-1BB in a reporter functional assay shown as signal-to-background ratio (S/B) (FIG. 7B).

FIG. 8 schematically depicts anti-4-1BB/anti-PD-1 bispecific binding molecules according to certain exemplary embodiments.

FIGS. 9A-7C depict the activity of anti-4-1BB/anti-PD-1 bispecific binding molecules [Constructs #1, 3, 5 and 6] versus control antibodies and isotype controls, in a T-cell activation assay (FIG. 9A), a mixed lymphocyte reaction (MLR; FIG. 9B) and a CD3-PBMC activation assay (FIG. 9C).

FIGS. 10A-10G depict the results of binding assays on:
human 300.19 pre-B-cells expressing either human PD-1 (FIG. 10A), cynomolgus monkey PD-1 (FIG. 10B), human 4-1BB (FIG. 10C) or cynomolgus monkey 4-1BB (FIG. 10D);
Jurkat T-cells expressing either human PD-1 (FIG. 10E) or human 4-1BB (FIG. 10F);
stimulated primary T-cells (FIG. 10G),
   using one of “Construct #3”, the anti-PD-1 arm alone (clone T5), or a control anti-4-1BB or anti-PD-1 antibody, in a dose-dependent manner.

FIGS. 11A-11B depict the effects in reporter assays (4-1BB reporter assay: FIG. 11A; PD-1 reporter assay: FIG. 11B) of the anti-4-1BB/anti-PD-1 bispecific binding molecule “Construct #3” versus control antibodies and isotype controls, shown as signal-to-noise ratio (S/N).

FIGS. 12A-12B depict the effects in an antibody-dependent cellular cytotoxicity (ADCC) assay of the anti-4-1BB/anti-PD-1 bispecific binding molecule “Construct #3” versus control antibodies (one control anti-4-1BB antibody [Ctrl anti-4-1BB Ab], FIG. 12A; two control anti-PD-1 antibodies [Ctrl anti-PD-1 Ab #1 and #2], FIG. 12B) and isotype controls.

FIGS. 13A-13B depict the effects in an antibody-dependent cellular phagocytosis (ADCP) assay of the anti-4-1BB/anti-PD-1 bispecific binding molecule “Construct #3” versus control antibodies (one control anti-4-1BB antibody [Ctrl anti-4-1BB Ab], FIG. 13A; two control anti-PD-1 antibodies [Ctrl anti-PD-1 Ab #1 and #2], FIG. 13B) and isotype controls.

FIGS. 14A-14B depict the effects in a complement-dependent cytotoxicity (CDC) assay of the anti-4-1BB/anti-PD-1 bispecific binding molecule “Construct #3” versus control antibodies (one control anti-4-1BB antibody [Ctrl anti-4-1BB Ab], FIG. 14A; two control anti-PD-1 antibodies [Ctrl anti-PD-1 Ab #1 and #2], FIG. 14B) and isotype controls.

FIGS. 15A-15C depict the effects in a T-cell activation (TCA) assay of the anti-4-1BB/anti-PD-1 bispecific binding molecule “Construct #3” versus control anti-4-1BB antibody [Ctrl anti-4-1BB Ab] and isotype controls. FIG. 15A shows the release of interferon-gamma [IFN-γ] from activated human T-cells; FIG. 15B shows the release of tumor necrosis factor-alpha [TNF-α] from activated human T-cells in a dose-dependent manner; FIG. 15C shows the release of interleukin-2 [IL-2] from activated cynomolgus monkey T-cells.

FIGS. 16A-16C depict the activity of the anti-4-1BB/anti-PD-1 bispecific binding molecule “Construct #3” versus the combination of a control anti-4-1BB antibody [Ctrl anti-4-1BB Ab] and one or two control anti-PD-1 antibodies [Ctrl anti-PD-1 Ab [#1 & #2]], and isotype controls, in a mixed lymphocyte reaction (MLR) assay. The release of interleukin-2 [IL-2] from the co-culture is depicted on FIG. 16A; FIG. 16B shows the release of interleukin-2 [IL-2] in a dose-dependent manner; FIG. 16C shows the release of tumor necrosis factor-alpha [TNF-α] in a dose-dependent manner.

FIGS. 17A-17B depict the activity of the anti-4-1BB/anti-PD-1 bispecific binding molecule “Construct #3” versus a control anti-4-1BB antibody [Ctrl anti-4-1BB Ab], a control anti-PD-1 antibody [Ctrl anti-PD-1 Ab], and combination thereof, and isotype controls, in a CD3-PBMC activation assay. The release of interferon-gamma [IFN-γ] (FIG. 17A) and of tumor necrosis factor-alpha [TNF-α] (FIG. 17B) from PBMC is depicted.

FIGS. 18A-18C depict the activity of the anti-4-1BB/anti-PD-1 bispecific binding molecule “Construct #3” versus a control anti-4-1BB antibody [Ctrl anti-4-1BB Ab], a control anti-PD-1 antibody [Ctrl anti-PD-1 Ab], and the combination thereof, and isotype controls, in a CD3-PBMC activation assay. The release of interleukin-2 [IL-2] (FIG. 18A), of tumor necrosis factor-alpha [TNF-α] (FIG. 18B), and of interleukin-5 [IL-5] (FIG. 18C) from PBMC in a dose-dependent manner is depicted.

FIGS. 19A-19C depict the reinvigoration of exhausted T-cells in a Modular IMmune In vitro Construct (MIMIC) CD8⁺ T-cell exhaustion assay, using the anti-4-1BB/anti-PD-1 bispecific binding molecule “Construct #3” versus one control anti-4-1BB antibody [Ctrl anti-4-1BB Ab], two control anti-PD-1 antibodies [Ctrl anti-PD-1 Ab #1 & #2], and combination thereof, and isotype controls. Also tested were two “Construct #3” mutants, one devoid of functional PD-1 arm [Construct #3 ΔPD-1] and one devoid of functional 4-1BB arm [Construct #3 Δ4-1BB]. FIG. 19A shows the total number of antigen-specific divided CD8⁺ T-cells at day 19 after treatment; FIG. 19B shows the secretion of interferon-gamma [IFN-γ] from reinvigorated CD8⁺ T-cells; FIG. 19C shows the secretion of tumor necrosis factor-alpha [TNF-α] from reinvigorated CD8⁺ T-cells.

FIG. 20 depicts the results of a regulatory T-cells (T_{reg}) suppression assay, using the anti-4-1BB/anti-PD-1 bispecific binding molecule “Construct #3” versus a control anti-4-1BB antibody [Ctrl anti-4-1BB Ab], and isotype controls, at several ratios of T effector cells [Tᵣₑₛₚ] to expanded T regulatory cells [eT_{reg}].

FIG. 21 depicts the results of an in vivo efficacy study in tumor-bearing mice (n=9 mice), using the anti-4-1BB/anti-PD-1 bispecific binding molecule “Construct #3” (at low or high dose) versus a control anti-4-1BB antibody [Ctrl anti-4-1BB Ab] (at 0.3 mpk), a control anti-PD-1 antibody [Ctrl anti-PD-1 Ab] (at 3 mpk), and combination thereof (at 0.3+3 mpk), and isotype control (at 3 mpk). DPI: days post-implant.

FIGS. 22A-22B depict the density of binding, in a dose-dependent manner, of anti-4-1BB/anti-PD-1 bispecific binding molecules “Construct #3” (FIG. 22A) and “Optimized Construct #3” (FIG. 22B), expressed as the number of molecules bound per μm² of Jurkat cells' surface expressing 4-1BB and PD-1. Controls include “Construct #3” and “Optimized Construct #3” mutants, either devoid of functional PD-1 arm [ΔPD-1], of functional 4-1BB arm [Δ4-1BB] or both [Δ4-1BB ΔPD-1].

FIGS. 23A-23B depict the effects in a T-cell activation (TCA) assay of the anti-4-1BB/anti-PD-1 bispecific binding molecules “Construct #3” and “Optimized Construct #3” versus a control anti-4-1BB antibody [Ctrl anti-4-1BB Ab] and isotype control. FIG. 23A shows the release of interferon-gamma [IFN-γ] from activated human T-cells; FIG. 23B shows the release of interleukin-2 [IL-2] from activated human T-cells.

FIGS. 24A-24B depict the activity of the anti-4-1BB/anti-PD-1 bispecific binding molecules “Construct #3” and “Optimized Construct #3” versus the combination of a control anti-4-1BB antibody and a control anti-PD-1 antibody [Ctrl anti-4-1BB Ab+Ctrl anti-PD-1 Ab], and isotype controls, in a mixed lymphocyte reaction (MLR) assay. FIG. 24A shows the release of interferon-gamma [IFN-γ] from the co-culture FIG. 24B shows the release of interleukin-2 [IL-2] from the co-culture.

FIG. 25 depicts the results of a surface plasmon resonance (SPR) binding assay on human 4-1BB and human PD-1, using “Optimized Construct #3”, or one of 4 masked version thereof (MC1-MC4), or a protease-activated version (i.e., demasked) of the masked compound ([MMP9-activated]), versus a control anti-4-1BB antibody [Ctrl anti-4-1BB Ab], a control anti-PD-1 antibody [Ctrl anti-PD-1 Ab], and isotype controls. Controls also include “Optimized Construct #3” mutants, either devoid of functional PD-1 arm [ΔT5_optimized], or partially or totally devoid of functional 4-1BB arm ([Δclone #2.1] or [Δclones #2.1 & #5.1], respectively).

FIGS. 26A-26D depict the results of a binding assay on human 300.19 pre-B-cells expressing either human 4-1BB (FIG. 26A), cynomolgus monkey 4-1BB (FIG. 26B), human PD-1 (FIG. 26C) or cynomolgus monkey PD-1 (FIG. 26D), using one of “Optimized Construct #3”, or one of 4 masked version thereof (MC1-MC4), or a protease-activated version (i.e., demasked) of the masked compound ([MMP9-activated]), in a dose-dependent manner.

FIG. 27 depicts the results of a binding assay on Jurkat/NF-κB-4-1BB cells, using “Optimized Construct #3”, or one of 4 masked version thereof (MC1-MC4), or a protease-activated version (i.e., demasked) of the masked compound ([MMP9-activated] or [uPa-activated]).

FIGS. 28A-28B depict the results of a binding assay on human Jurkat T-cells expressing either human 4-1BB (FIG. 28A) or human PD-1 (FIG. 28B), using one of “Optimized Construct #3”, or one of 4 masked version thereof (MC1-MC4), or a protease-activated version (i.e., demasked) of the masked compound ([MMP9-activated]), in a dose-dependent manner.

FIG. 29 depicts the results of a luciferase-based reporter assay, using “Optimized Construct #3”, or one of 4 masked version thereof (MC1-MC4), or a protease-activated version (i.e., demasked) of the masked compound ([MMP9-activated] or [uPa-activated]).

FIGS. 30A-30B depict the results of a luciferase-based reporter assay on 4-1BB⁺ Jurkat T-cells (FIG. 30A) or PD-1⁺ Jurkat T-cells (FIG. 30B), shown as signal-to-noise ratio (S/N), using “Optimized Construct #3”, or one of 4 masked version thereof (MC1-MC4), or a protease-activated version (i.e., demasked) of the masked compound ([MMP9-activated]).

FIG. 31 depicts the results of a T-cell activation (TCA) assay, using “Optimized Construct #3”, or one of 4 masked version thereof (MC1-MC4), or a protease-activated version (i.e., demasked) of the masked compound ([MMP9-activated] or [uPa-activated]).

FIGS. 32A-32D depict the results of a murine T-cell activation (TCA) assay using “Optimized Construct #3”, or one of 4 masked version thereof (MC1-MC4), or a protease-activated version (i.e., demasked) of the masked compound ([MMP9-activated]) versus isotype control. Figures show the release of interleukin-4 ([IL-4]; FIG. 32A); of interleukin-2 ([IL-2]; FIG. 32B); of interferon-gamma ([IFN-γ]; FIG. 32C); and of tumor necrosis factor-alpha ([TNF-α]; FIG. 32D).

FIGS. 33A-33C depict the activity of “Optimized Construct #3”, or one of 4 masked version thereof (MC1-MC4), or a protease-activated version (i.e., demasked) of the masked compound ([MMP9-activated]), in a mixed lymphocyte reaction (MLR) assay. FIG. 33A shows the release of interleukin-2 [IL-2] from the co-culture; FIG. 33B shows donor-dependent activity expressed as the EC₅₀ for interleukin-2 [IL-2] release; FIG. 33C shows donor-dependent activity expressed as the EC₅₀ for interferon-gamma [IFN-γ] release.

FIGS. 34A-34E depict the activity of “Optimized Construct #3”, or one of 4 masked version thereof (MC1-MC4), or a protease-activated version (i.e., demasked) of the masked compound ([MMP9-activated]), in a CD3-PBMC activation assay. FIG. 34A shows the release of interleukin-2 [IL-2] from PBMC; FIG. 34B shows donor-dependent activity expressed as the EC₅₀ for interleukin-2 [IL-2] release; FIG. 34C shows donor-dependent activity expressed as the EC₅₀ for interferon-gamma [IFN-γ] release; FIG. 34D shows donor-dependent activity expressed as the EC₅₀ for tumor necrosis factor-alpha [TNF-α] release; FIG. 34E shows donor-dependent activity expressed as the EC₅₀ for interleukin-5 [IL-5] release.

FIG. 35 depicts the results of an in vivo efficacy study in tumor-bearing mice (n=11 mice/group), using the anti-4-1BB/anti-PD-1 bispecific binding molecule “Optimized Construct #3” (at low, medium, or high dose), and the masked version thereof “MC1” (at the same low, medium, and high doses), versus isotype control [iso ctrl] at high dose. Data are shown as the log 2 median tumor volume (in mm³) of all mice per group. DPI: days post-implant.

### DETAILED DESCRIPTION

Before the present disclosure is described, it is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

### Definitions

Unless defined otherwise, all technical and scientific terms used herein have the same meaning as commonly understood by one of ordinary skill in the art to which this disclosure belongs.

As used herein, the term “about”, when used in reference to a particular recited numerical value, means that the value may vary from the recited value by no more than 5%, preferably no more than 2%, and more preferably no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice of the present disclosure, exemplary methods and materials are now described. All publications mentioned herein are incorporated herein by reference to describe in their entirety.

The term “PD-1” refers to the programmed death-1 protein, a T-cell co-inhibitor, also known as CD279. Unless specified as being from a non-human species, the term “PD-1” means human PD-1. PD-1 is a member of the CD28/CTLA-4/ICOS family of T-cell co-inhibitors. PD-1 is a 288-amino acid protein with an extracellular N-terminal domain which is IgV-like, a transmembrane domain and an intracellular domain containing an immunoreceptor tyrosine-based inhibitory (ITIM) motif and an immunoreceptor tyrosine-based switch (ITSM) motif (Chattopadhyay et al., Immunol Rev. 2009 May;229(1):356-86). The PD-1 receptor has two ligands, PD-ligand-1 (PD-L1) and PD-L2. An exemplary amino acid sequence of PD-1 is set forth in SEQ ID NO: 42, which corresponds to human PD-1 (hPD-1) with GenBank accession number NP 005009.2.

SEQ ID NO: 42
MQIPQAPWPVVWAVLQLGWRPGWFLDSPDRPWNPPTFSPALLVVTEGDN
ATFTCSFSNTSESFVLNWYRMSPSNQTDKLAAFPEDRSQPGQDCRFRVT
QLPNGRDFHMSVVRARRNDSGTYLCGAISLAPKAQIKESLRAELRVTER
RAEVPTAHPSPSPRPAGQFQTLVVGVVGGLLGSLVLLVWVLAVICSRAA
RGTIGARRTGQPLKEDPSAVPVFSVDYGELDFQWREKTPEPPVPCVPEQ
TEYATIVFPSGMGTSSPARRGSADGPRSAQPLRPEDGHCSWPL

The term “PD-L1” refers to the ligand of the PD-1 receptor also known as CD274 and B7H1. PD-L1 is a 290-amino acid protein with an extracellular IgV-like domain, a transmembrane domain and a highly conserved intracellular domain of approximately 30 amino acids. PD-L1 is constitutively expressed on many cells such as antigen presenting cells (e.g., dendritic cells, macrophages, and B-cells) and on hematopoietic and non-hematopoietic cells (e.g., vascular endothelial cells, pancreatic islets, and sites of immune privilege). PD-L1 is also expressed on a wide variety of tumors and virally-infected cells, and is a component of the immunosuppressive milieu (Ribas, N Engl J Med. 2012 Jun. 28; 366(26):2517-9). An exemplary amino acid sequence of PD-L1 is set forth in SEQ ID NO: 43, which corresponds to human PD-L1 (hPD-L1) with GenBank accession number NP 054862.1.

SEQ ID NO: 43
MRIFAVFIFMTYWHLLNAFTVTVPKDLYVVEYGSNMTIECKFPVEKQLDL
AALIVYWEMEDKNIIQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGKTTTTNSKREEKLFNVTSTLRIN
TTTNEIFYCTFRRLDPEENHTAELVIPELPLAHPPNERTHLVILGAILLC
LGVALTFIFRLRKGRMMDVKKCGIQDTNSKKQSDTHLEET

As used herein, the term “4-1BB” or “CD137” refers to a surface glycoprotein that belongs to the tumor necrosis factor receptor family (TNFRSF9). Its expression is induced on activation on a number of leukocyte types. 4-1BB becomes expressed on primed T- and natural killer (NK) cells, which on ligation provides powerful costimulatory signals. Perturbation of 4-1BB by 4-1BBL or agonist monoclonal antibodies on activated CD8⁺ T-cells protects such antigen-specific cytotoxic T-lymphocytes from apoptosis, enhances effector functionalities and favors persistence and memory differentiation. 4-1BB is transiently expressed on activated T-cells that have encountered cognate antigen, activated NK cells or mature dendritic cells (DCs). A unique functional ligand has been identified for 4-1BB, 4-1BBL, expressed on the surface of professional antigen-presenting cells such as DCs, macrophages and B-cells. 4-1BB trimerization leads to 4-1BBL receptor clustering and TRAFs-mediated activation of NF-κB and MAPK intracellular signaling cascades, ultimately leading to cell activation, proliferation and survival. On T-cells, T-cell receptor (TCR) stimulation and subsequent CD3 signaling induce transient expression of 4-1BB that on ligation with agonist antibodies or the natural ligand favors Th1 responses. In addition to inducing effector cytokine production, 4-1BB co-stimulation favors T-cell memory and effector differentiation, protects T-cells from apoptosis, changes the mitochondrial metabolism to increase T-cell respiratory capacities and induces overall DNA demethylation and chromatin reprogramming. An exemplary amino acid sequence of 4-1BB is set forth in SEQ ID NO: 13, which corresponds to human 4-1BB (h4-1BB) with UniProt accession number Q07011. 4-1BB contains four cysteine-rich domains (CRD) in the N-terminal extracellular region: CRD1 (amino acid residues 24-45 of SEQ ID NO: 13); CRD2 (amino acid residues 47-86 of SEQ ID NO: 13); CRD3 (amino acid residues 87-118 of SEQ ID NO: 13); and CRD4 (amino acid residues 119-159 of SEQ ID NO: 13).

SEQ ID NO: 13
MGNSCYNIVATLLLVLNFERTRSLQDPCSNCPAGTFCDNNRNQICSPCPP
NSFSSAGGQRTCDICRQCKGVFRTRKECSSTSNAECDCTPGFHCLGAGCS
MCEQDCKQGQELTKKGCKDCCFGTFNDQKRGICRPWTNCSLDGKSVLVNG
TKERDVVCGPSPADLSPGASSVTPPAPAREPGHSPQIISFFLALTSTALL
FLLFFLTLRFSVVKRGRKKLLYIFKQPFMRPVQTTQEEDGCSCRFPEEEE
GGCEL

As used herein, the term “T-cell co-inhibitor” refers to a receptor expressed on a T-cell and/or to such receptor's ligand, which modulates the immune response via T-cell receptor (TCR) signaling suppression. The term “T-cell co-inhibitor”, also known as “T-cell co-signaling molecule”, includes, but is not limited to, PD-1; lymphocyte activation gene 3 protein (LAG-3, also known as CD223); cytotoxic T-lymphocyte antigen-4 (CTLA-4); B and T lymphocyte attenuator (BTLA); 2B4; T-cell immunoglobulin and mucin-3 (TIM-3); T-cell immunoreceptor with immunoglobulin and ITIM (TIGIT; also known as VSIG9); leucocyte associated immunoglobulin-like receptor-1 (LAIR-1; also known as CD305); V-domain Ig suppressor of T-cell activation (VISTA); PD-L1; PD-L2; CEACAM; B7-H3; B7-H4; KIR; A2aR; GAL9; and TGFR. For a review of T-cell co-stimulation and co-inhibition, see Chen & Flies (Nat Rev Immunol. 2013 April; 13(4):227-42).

As used herein, the term “T-cell co-stimulator” refers to a receptor expressed on a T-cell and/or to such receptor's ligand, which modulates the immune response via T-cell receptor (TCR) signaling activation. The term “T-cell co-stimulator” includes, but is not limited to, CD28; inducible T-cell co-stimulator (ICOS); OX40; CD27; 4-1BB (also known as CD137); death receptor 3 (DR3); B7; CD226; CRTAM; glucocorticoid-induced TNFR-related protein (GITR); CD30; CD2; herpes virus entry mediator (HVEM); BAFFR; BAFF; and Light. For a review of T-cell co-stimulation and co-inhibition, see Chen & Flies (Nat Rev Immunol. 2013 April;13(4):227-42).

The term “antigen-binding protein”, as used herein, refers to a protein capable of binding specifically to at least one target via at least one immunoglobulin (Ig) variable domain. Examples for antigen-binding proteins include, but are not limited to, antibodies or fragments thereof, single domain antibodies, Fab fragments, immunoglobulin single variable domains (ISV), and combinations thereof. Antigen-binding proteins may be of non-human (e.g., murine) or human origin. If such antigen-binding proteins are of non-human (e.g., murine) origin, these may be “humanized” to decrease immunogenicity or to increase stability.

The term “antibody”, as used herein, is intended to refer to immunoglobulin (Ig) molecules comprised of four polypeptide chains, two heavy (H) chains and two light (L) chains inter-connected by disulfide bonds (i.e., “full antibody molecules”), as well as multimers thereof (e.g., IgM) or antigen-binding fragments thereof. Each heavy chain is comprised of a heavy chain variable region (“HCVR” or “V_{H}”) and a heavy chain constant region (“HCCR” or “C_{H}”; comprised of domains C_{H}1, C_{H}2 and C_{H}3). Each light chain is comprised of a light chain variable region (“LCVR or “V_{L}”) and a light chain constant region (“LCCR” or “C_{L}”). The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In certain embodiments of the disclosure, the FRs of the antibody (or antigen-binding fragment thereof) may be identical to the human germline sequences, or may be naturally or artificially modified. An amino acid consensus sequence may be defined based on a side-by-side analysis of two or more CDRs.

The term “immunoglobulin single variable domain” (ISV), interchangeably used with “single variable domain”, defines immunoglobulin molecules wherein the antigen-binding site is present on, and formed by, a single immunoglobulin domain. This sets immunoglobulin single variable domains apart from “conventional” immunoglobulins (e.g., monoclonal antibodies) or their fragments (such as Fab, Fab′, F(ab′)₂, scFv, di-scFv), wherein two immunoglobulin domains, in particular two variable domains, interact to form an antigen-binding site. Typically, in conventional immunoglobulins, a heavy chain variable domain (V_{H}) and a light chain variable domain (V_{L}) interact to form an antigen binding site. In this case, the complementarity determining regions (CDRs) of both V_{H} and V_{L} will contribute to the antigen binding site, i.e., a total of 6 CDRs will be involved in antigen-binding site formation. In view of the above definition, the antigen-binding domain of a conventional 4-chain antibody (such as an IgG, IgM, IgA, IgD or IgE molecule, known in the art) or of a Fab fragment, a F(ab′)₂ fragment, an Fv fragment such as a disulfide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associating) immunoglobulin domains such as light and heavy chain variable domains, i.e., by a V_{H}-V_{L} pair of immunoglobulin domains, which jointly bind to an epitope of the respective antigen.

In contrast, immunoglobulin single variable domains are capable of specifically binding to an epitope of the antigen without pairing with an additional immunoglobulin variable domain. The binding site of an immunoglobulin single variable domain is formed by a single V_{H}, a single V_{HH} or single V_{L} domain. Hence, the antigen binding site of an immunoglobulin single variable domain is formed by no more than three CDRs. As such, the single variable domain may be a light chain variable domain sequence (e.g., a V_{L}-sequence) or a suitable fragment thereof, or a heavy chain variable domain sequence (e.g., a V_{H}-sequence or V_{HH} sequence) or a suitable fragment thereof, as long as it is capable of forming a single antigen-binding unit (i.e., a functional antigen-binding unit that essentially consists of the single variable domain, such that the single antigen-binding domain does not need to interact with another variable domain to form a functional antigen-binding unit).

An immunoglobulin single variable domain (ISV) can for example be a heavy chain ISV, such as a V_{H}, V_{HH}, including a camelized V_{H} or humanized V_{HH}. In one embodiment, it is a V_{HH}, including a camelized V_{H} or humanized V_{HH}. Heavy chain ISVs can be derived from a conventional four-chain antibody or from a heavy chain antibody. For example, the immunoglobulin single variable domain may be a (single) domain antibody (or an amino acid sequence that is suitable for use as a single domain antibody), a “dAb” or dAb (or an amino acid sequence that is suitable for use as a dAb) or a Nanobody® ISV (as defined herein and including, but not limited to, a V_{HH}); other single variable domains, or any suitable fragment of any one thereof. In particular, the immunoglobulin single variable domain may be a Nanobody® ISV (such as a V_{HH}, including a humanized V_{HH} or camelized V_{H}) or a suitable fragment thereof.

“V_{HH} domains”, also known as “V_{HH}S”, “V_{HH} antibody fragments”, and “V_{HH} antibodies”, have originally been described as the antigen binding immunoglobulin variable domain of “heavy chain antibodies” (i.e., of “antibodies devoid of light chains”; Hamers-Casterman et al., Nature. 1993 Jun. 3; 363(6428):446-8). The term “V_{HH} domain” has been chosen in order to distinguish these variable domains from the heavy chain variable domains that are present in conventional 4-chain antibodies (which are referred to herein as “V_{H} domains”) and from the light chain variable domains that are present in conventional 4-chain antibodies (which are referred to herein as “V_{L} domains”). For a further description of V_{HH}'s, reference is made to the review article by Muyldermans (J Biotechnol. 2001 June;74(4):277-302). For the term “dAb's” and “domain antibody”, reference is for example made to Ward et al. (Nature. 1989 Oct. 12;341(6242):544-6), to Holt et al. (Trends Biotechnol. 2003 November; 21(11):484-90); as well as to, for example, WO 2004/068820, WO 2006/030220, WO 2006/003388 and other published patent applications of Domantis Ltd. It should also be noted that, although less preferred because they are not of mammalian origin, single variable domains can be derived from certain species of shark (for example, the so-called “IgNAR domains”, see for example WO 2005/18629).

Typically, the generation of immunoglobulins involves the immunization of experimental animals, fusion of immunoglobulin producing cells to create hybridomas and screening for the desired specificities. Alternatively, immunoglobulins can be generated by screening of naïve, immune or synthetic libraries e.g., by phage display. The generation of immunoglobulin sequences, such as V_{HH}s, has been described extensively in various publications, among which WO 1994/04678, Hamers-Casterman et al. (Nature. 1993 Jun. 3; 363(6428):446-8) and Muyldermans (J Biotechnol. 2001 June;74(4):277-302). In these methods, camelids are immunized with the target antigen in order to induce an immune response against said target antigen. The repertoire of V_{HH}s obtained from said immunization is further screened for V_{HH}s that bind the target antigen. In these instances, the generation of antibodies requires purified antigen for immunization and/or screening. Antigens can be purified from natural sources, or in the course of recombinant production. Immunization and/or screening for immunoglobulin sequences can be performed using peptide fragments of such antigens.

Immunoglobulin sequences of different origin, comprising mouse, rat, rabbit, donkey, human and camelid immunoglobulin sequences can be used herein. Also, fully human, humanized or chimeric sequences can be used in the method described herein. For example, camelid immunoglobulin sequences and humanized camelid immunoglobulin sequences, or camelized domain antibodies, e.g., camelized dAb as described by Ward et al. (Nature. 1989 Oct. 12; 341(6242):544-6), WO 1994/04678, and Davis et al. (FEBS Lett. 1994 Feb. 21; 339(3):285-90; and Protein Eng. 1996 June;9(6):531-7) can be used herein. Moreover, the ISVs can be fused to form a multivalent and/or multispecific construct (for multivalent and multispecific polypeptides containing one or more V_{HH} domains and their preparation, reference is also made to Conrath et al. (J Biol Chem. 2001 Mar. 9; 276(10):7346-50) as well as to, for example, WO 1996/34103 and WO 1999/23221).

A “humanized V_{HH}” comprises an amino acid sequence that corresponds to the amino acid sequence of a naturally occurring V_{HH} domain, but that has been “humanized”, i.e., by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g., indicated above). This can be performed in a manner known per se, which will be clear to the skilled person, for example on the basis of the prior art (e.g., WO 2008/020079). Again, it should be noted that such humanized V_{HH}s can be obtained in any suitable manner known per se and thus are not strictly limited to polypeptides that have been obtained using a polypeptide that comprises a naturally occurring V_{HH} domain as a starting material.

A “camelized V_{H}” comprises an amino acid sequence that corresponds to the amino acid sequence of a naturally occurring V_{H} domain, but that has been “camelized”, i.e., by replacing one or more amino acid residues in the amino acid sequence of a naturally occurring V_{H} domain from a conventional 4-chain antibody by one or more of the amino acid residues that occur at the corresponding position(s) in a V_{HH} domain of a (camelid) heavy chain antibody. This can be performed in a manner known per se, which will be clear to the skilled person, for example on the basis of the description in the prior art (e.g., Davies et al., FEBS Lett. 1994 Feb. 21; 339(3):285-90; Davies et al., Biotechnology (NY). 1995 May; 13(5):475-9; Davies et al., Protein Eng. 1996 June;9(6):531-7; and Riechmann et al., J Immunol Methods. 1999 Dec. 10; 231(1-2):25-38). Such “camelizing” substitutions are inserted at amino acid positions that form and/or are present at the V_{H}-V_{L} interface, and/or at the so-called Camelidae hallmark residues, as defined herein (see for example WO 1994/04678 and Davies et al. (1994 and 1996, supra)). In some embodiments, the V_{H} sequence that is used as a starting material or starting point for generating or designing the camelized V_{H} is a V_{H} sequence from a mammal, such as the V_{H} sequence of a human being, such as a V_{H}3 sequence. However, it should be noted that such camelized V_{H} can be obtained in any suitable manner known per se and thus are not strictly limited to polypeptides that have been obtained using a polypeptide that comprises a naturally occurring V_{H} domain as a starting material.

The structure of an immunoglobulin single variable domain (ISV) sequence can be considered to be comprised of four framework regions (“FRs”), which are referred to in the art and herein as “framework region 1” (“FR1”); “framework region 2” (“FR2”); “framework region 3” (“FR3”); and “framework region 4” (“FR4”), respectively; which framework regions are interrupted by three complementary determining regions (“CDRs”), which are referred to in the art and herein as “Complementarity Determining Region 1” (“CDR1”); “Complementarity Determining Region 2” (“CDR2”); and “Complementarity Determining Region 3” (“CDR3”), respectively. In such an immunoglobulin sequence, the framework sequences may be any suitable framework sequences, and examples of suitable framework sequences will be clear to the skilled person, for example on the basis the standard handbooks and the further disclosure and prior art mentioned herein. The framework sequences are (a suitable combination of) immunoglobulin framework sequences or framework sequences that have been derived from immunoglobulin framework sequences (for example, by humanization or camelization). For example, the framework sequences may be framework sequences derived from a light chain variable domain (e.g., a V_{L}-sequence) and/or from a heavy chain variable domain (e.g., a V_{H}-sequence or V_{HH} sequence). In one particular aspect, the framework sequences are either framework sequences that have been derived from a V_{HH}-sequence (in which said framework sequences may optionally have been partially or fully humanized) or are conventional V_{H} sequences that have been camelized (as defined herein). In particular, the framework sequences present in the ISV sequence described herein may contain one or more of hallmark residues (as defined herein), such that the ISV sequence is a Nanobody® ISV, such as e.g., a V_{HH}, including a humanized V_{HH} or camelized V_{H}. Non-limiting examples of (suitable combinations of) such framework sequences will become clear from the further disclosure herein.

The total number of amino acid residues in a V_{H} domain and a V_{HH} domain will usually be in the range of from 110 to 120, often between 112 and 115. It should however be noted that smaller and longer sequences may also be suitable for the purposes described herein. However, it should be noted that the ISVs described herein is not limited as to the origin of the ISV sequence (or of the nucleotide sequence used to express it), nor as to the way that the ISV sequence or nucleotide sequence is (or has been) generated or obtained. Thus, the ISV sequences may be naturally occurring sequences (from any suitable species) or synthetic or semi-synthetic sequences. In a specific but non-limiting aspect, the ISV sequence is a naturally occurring sequence (from any suitable species) or a synthetic or semi-synthetic sequence, including but not limited to “humanized” (as defined herein) immunoglobulin sequences (such as partially or fully humanized mouse or rabbit immunoglobulin sequences, and in particular partially or fully humanized V_{HH} sequences), “camelized” (as defined herein) immunoglobulin sequences (and in particular camelized V_{H} sequences), as well as ISVs that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing. Similarly, nucleotide sequences may be naturally occurring nucleotide sequences or synthetic or semi-synthetic sequences, and may for example be sequences that are isolated by PCR from a suitable naturally occurring template (e.g., DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

Generally, Nanobody® ISVs (in particular V_{HH} sequences, including (partially) humanized V_{HH} sequences and camelized V_{H} sequences) can be characterized by the presence of one or more “hallmark residues” (as described herein) in one or more of the framework sequences (again as further described herein). Thus, generally, a Nanobody® ISV can be defined as an immunoglobulin sequence with the (general) structure FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, a Nanobody® ISV can be an immunoglobulin sequence with the (general) structure FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3, respectively, and in which the framework sequences are as further defined herein.

More particularly, a Nanobody® ISV can be an immunoglobulin sequence with the (general) structure FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3, respectively, and in which one or more of the amino acid residues at positions 11, 37, 44, 45, 47, 83, 84, 103, 104 and 108 according to the Kabat numbering are chosen from the Hallmark residues mentioned in Table 1 below.

TABLE 1
hallmark residues in Nanobody ® ISVs

| Position | Human V_{H}3 | Hallmark Residues |
|---|---|---|
| 11 | L, V; predominantly L | L, S, V, M, W, F, T, Q, E, A, R, G, K, Y, N, P, I; preferably L |
| 37 | V, I, F; usually V | F⁽¹⁾, Y, V, L, A, H, S, I, W, C, N, G, D, T, P, preferably F⁽¹⁾ |
| | | or Y |
| 44⁽⁸⁾ | G | E⁽³⁾, Q⁽³⁾, G⁽²⁾, D, A, K, R, L, P, S, V, H, T, N, W, M, I; |
| | | preferably G⁽²⁾, E⁽³⁾ or Q⁽³⁾; most preferably G⁽²⁾ or Q⁽³⁾. |
| 45⁽⁸⁾ | L | L⁽²⁾, R⁽³⁾, P, H, F, G, Q, S, E, T, Y, C, I, D, V; preferably L⁽²⁾ |
| | | or R⁽³⁾ |
| 47⁽⁸⁾ | W, Y | F⁽¹⁾, L⁽¹⁾ or W⁽²⁾ G, I, S, A, V, M, R, Y, E, P, T, C, H, K, Q, |
| | | N, D; preferably W⁽²⁾, L⁽¹⁾ or F⁽¹⁾ |
| 83 | R or K; usually R | R, K⁽⁵⁾, T, E⁽⁵⁾, Q, N, S, I, V, G, M, L, A, D, Y, H; preferably |
| | | K or R; most preferably K |
| 84 | A, T, D; | P⁽⁵⁾, S, H, L, A, V, I, T, F, D, R, Y, N, Q, G, E; preferably P |
| | predominantly A | |
| 103 | W | W⁽⁴⁾, R⁽⁶⁾, G, S, K, A, M, Y, L, F, T, N, V, Q, P⁽⁶⁾, E, C; |
| | | preferably W |
| 104 | G | G, A, S, T, D, P, N, E, C, L; preferably G |
| 108 | L, M or T; | Q, L⁽⁷⁾, R, P, E, K, S, T, M, A, H; preferably Q or L⁽⁷⁾ |
| | predominantly L | |

Notes:
⁽¹⁾In particular, but not exclusively, in combination with KERE or KQRE at positions 43-46.
⁽²⁾Usually as GLEW at positions 44-47.
⁽³⁾Usually as KERE or KQRE at positions 43-46, e.g. as KEREL, KEREF, KQREL, KQREF, KEREG, KQREW or KQREG at positions 43-47. Alternatively, also sequences such as TERE (for example TEREL), TQRE (for example TQREL), KECE (for example KECEL or KECER), KQCE (for example KQCEL), RERE (for example REREG), RQRE (for example RQREL, RQREF or RQREW), QERE (for example QEREG), QQRE, (for example QQREW, QQREL or QQREF), KGRE (for example KGREG), KDRE (for example KDREV) are possible. Some other possible, but less preferred sequences include for example DECKL and NVCEL.
⁽⁴⁾With both GLEW at positions 44-47 and KERE or KQRE at positions 43-46.
⁽⁵⁾Often as KP or EP at positions 83-84 of naturally occurring V_{HH} domains.
⁽⁶⁾In particular, but not exclusively, in combination with GLEW at positions 44-47.
⁽⁷⁾With the proviso that when positions 44-47 are GLEW, position 108 is always Q in (non-humanized) V_{HH} sequences that also contain a W at 103.
⁽⁸⁾The GLEW group also contains GLEW-like sequences at positions 44-47, such as for example GVEW, EPEW, GLER, DQEW, DLEW, GIEW, ELEW, GPEW, EWLP, GPER, GLER and ELEW.

In some embodiments, the immunoglobulin single variable domain has certain amino acid substitutions in the framework regions effective in preventing or reducing binding of so-called “pre-existing antibodies” to the polypeptides. ISVs in which (i) the amino acid residue at position 112 is one of K or Q; and/or (ii) the amino acid residue at position 89 is T; and/or (iii) the amino acid residue at position 89 is L and the amino acid residue at position 110 is one of K or Q and (iv) in each of cases (i) to (iii), the amino acid at position 11 is preferably V have been described in WO 2015/173325.

The immunoglobulin single variable domains (ISVs) may form part of a protein or polypeptide, which may comprise or essentially consist of one or more (at least one) immunoglobulin single variable domains and which may optionally further comprise one or more further amino acid sequences (all optionally linked via one or more suitable linkers). The term “immunoglobulin single variable domain” may also encompass such polypeptides. The one or more immunoglobulin single variable domains may be used as a binding unit in such a protein or polypeptide, which may optionally contain one or more further amino acids that can serve as a binding unit, so as to provide a monovalent, multivalent or multispecific polypeptide of the invention, respectively (for multivalent and multispecific polypeptides containing one or more V_{HH} domains and their preparation, reference is also made to Conrath et al. (J Biol Chem. 2001 Mar. 9; 276(10):7346-50), as well as to for example WO 1996/34103, WO 1999/23221 and WO 2010/115998).

The polypeptides may comprise or essentially consist of one immunoglobulin single variable domain, as outlined above. Such polypeptides are also referred to herein as “monovalent” polypeptides. The term “multivalent” indicates the presence of multiple ISVs in a polypeptide. In one embodiment, the polypeptide is “bivalent”, i.e., comprises or consists of two ISVs. In one embodiment, the polypeptide is “trivalent”, i.e., comprises or consists of three ISVs. In another embodiment, the polypeptide is “tetravalent”, i.e., comprises or consists of four ISVDs. The polypeptide can thus be “bivalent”, “trivalent”, “tetravalent”, “pentavalent”, “hexavalent”, “heptavalent”, “octavalent”, “nonavalent”, etc., i.e., the polypeptide comprises or consists of two, three, four, five, six, seven, eight, nine, etc., ISVs, respectively. In one embodiment, the multivalent ISV polypeptide is bivalent. In one embodiment, the multivalent ISV polypeptide is trivalent. In another embodiment the multivalent ISV polypeptide is tetravalent. In still another embodiment, the multivalent ISV polypeptide is pentavalent.

The multivalent ISV polypeptide can also be monospecific or multispecific. The term “multispecific” refers to binding to multiple different target molecules (also referred to as antigens). The multivalent ISV polypeptide can thus be “bispecific”, “trispecific”, “tetraspecific”, etc., i.e., can bind to two, three, four, etc., different target molecules, respectively. For example, the polypeptide may be bispecific-trivalent, such as a polypeptide comprising or consisting of three ISVs, wherein two ISVs bind to a first target and one ISV binds to a second target different from the first target. In another example, the polypeptide may be trispecific-tetravalent, such as a polypeptide comprising or consisting of four ISVs, wherein one ISV binds to a first target, two ISVs bind to a second target different from the first target and one ISV binds to a third target different from the first and the second target. In still another example, the polypeptide may be trispecific-pentavalent, such as a polypeptide comprising or consisting of five ISVs, wherein two ISVs bind to a first target, two ISVs bind to a second target different from the first target and one ISV binds to a third target different from the first and the second target.

In one embodiment, the multivalent ISV polypeptide can further be monoparatopic or multiparatopic. The term “multiparatopic” refers to binding to multiple different epitopes on the same target molecules (also referred to as antigens). The multivalent ISV polypeptide can thus be “biparatopic”, “triparatopic”, etc., i.e., can bind to two, three, etc., different epitopes on the same target molecules, respectively.

In another aspect, the polypeptide of the invention that comprises or essentially consists of one or more immunoglobulin single variable domains (or suitable fragments thereof), may further comprise one or more other groups, residues, moieties or binding units. Such further groups, residues, moieties, binding units or amino acid sequences may or may not provide further functionality to the immunoglobulin single variable domain (and/or to the polypeptide in which it is present) and may or may not modify the properties of the immunoglobulin single variable domain. For example, such further groups, residues, moieties or binding units may be one or more additional amino acids, such that the compound, construct or polypeptide is a (fusion) protein or (fusion) polypeptide. In a preferred but non-limiting aspect, said one or more other groups, residues, moieties or binding units are immunoglobulins. Even more preferably, said one or more other groups, residues, moieties or binding units are chosen from the group consisting of domain antibodies, amino acids that are suitable for use as a domain antibody, single domain antibodies, amino acids that are suitable for use as a single domain antibody, “dAb”s, amino acids that are suitable for use as a dAb, or Nanobody® ISVs. Alternatively, such groups, residues, moieties or binding units may for example be chemical groups, residues, moieties, which may or may not by themselves be biologically and/or pharmacologically active. For example, and without limitation, such groups may be linked to the one or more immunoglobulin single variable domain so as to provide a “derivative” of the immunoglobulin single variable domain. In another embodiment, said further residues may be effective in preventing or reducing binding of so-called “pre-existing antibodies” to the polypeptides. For this purpose, the polypeptides and constructs may contain a C-terminal extension (X)ₙ (in which n is 1 to 10, preferably 1 to 5, such as 1, 2, 3, 4 or 5 (and preferably 1 or 2, such as 1); and each X is an (preferably naturally occurring) amino acid residue that is independently chosen, and preferably independently chosen from the group consisting of alanine (A), glycine (G), valine (V), leucine (L) or isoleucine (I), for which reference is made to WO 2012/175741. Accordingly, the polypeptide may further comprise a C-terminal extension (X)ₙ, in which n is 1 to 5, such as 1, 2, 3, 4 or 5, and in which X is a naturally occurring amino acid, preferably no cysteine.

In the polypeptides described above, the one or more immunoglobulin single variable domains and the one or more groups, residues, moieties or binding units may be linked directly to each other and/or via one or more suitable linkers or spacers. For example, when the one or more groups, residues, moieties or binding units are amino acids, the linkers may also be an amino acid, so that the resulting polypeptide is a fusion protein or fusion polypeptide. As used herein, the term “linker” denotes a peptide that fuses together two or more ISVs into a single molecule. The term also extends to a peptide that fuses together any two amino acid sequences in a so-called “fusion protein” or “fusion polypeptide”, like, e.g., an ISV and one or more other groups, residues, moieties or binding units as defined above.

The use of linkers to connect two or more (poly)peptides is well known in the art. One often used class of peptidic linker are known as the “Gly-Ser” or “GS” linkers. These are linkers that essentially consist of glycine (G) and serine (S) residues, and usually comprise one or more repeats of a peptide motif such as the GGGGS (SEQ ID NO: 26) motif (for example, having the formula (Gly-Gly-Gly-Gly-Ser)ₙ in which n may be 1, 2, 3, 4, 5, 6, 7 or more). Some often-used examples of such GS linkers are 9GS linkers (GGGGSGGGS, SEQ ID NO: 29), 15GS linkers (n=3) and 35GS linkers (n=7). Reference is for example made to Chen et al. (Adv Drug Deliv Rev. 2013 October;65(10):1357-69) and Klein et al. (Protein Eng Des Sel. 27(10):325-30). Further exemplary peptidic linkers are shown in Table 2 below.

TABLE 2
linker sequences

| | SEQ | |
|---|---|---|
| | ID | |
| Name | NO | Amino acid sequence |
| 3A linker | — | AAA |
| 5GS linker | 26 | GGGGS |
| 7GS linker | 27 | SGGSGGS |
| 8GS linker | 28 | GGGGSGGS |
| 9GS linker | 29 | GGGGSGGGS |
| 10GS linker | 30 | GGGGSGGGGS |
| 15GS linker | 31 | GGGGSGGGGSGGGGS |
| 18GS linker | 32 | GGGGSGGGGSGGGGSGGS |
| 20GS linker | 33 | GGGGSGGGGSGGGGSGGGGS |
| 25GS linker | 34 | GGGGSGGGGSGGGGSGGGGSGGGG |
| | | S |
| 30GS linker | 35 | GGGGSGGGGSGGGGSGGGGSGGGG |
| | | SGGGGS |
| 35GS linker | 36 | GGGGSGGGGSGGGGSGGGGSGGGG |
| | | SGGGGSGGGGS |
| 40GS linker | 37 | GGGGSGGGGSGGGGSGGGGSGGGG |
| | | SGGGGSGGGGSGGGGS |
| G1 hinge | 38 | EPKSCDKTHTCPPCP |
| 9GS-G1 hinge | 39 | GGGGSGGGSEPKSCDKTHTCPPCP |
| Llama upper | 40 | EPKTPKPQPAAA |
| long hinge | | |
| region | | |
| G3 hinge | 41 | ELKTPLGDTTHTCPRCPEPKSCDT |
| | | PPPCPRCPEPKSCDTPPPCPRCPE |
| | | PKSCDTPPPCPRCP |

In all antigen-binding proteins disclosed herein, substitution of one or more CDR residues or omission of one or more CDRs is also possible. Antibodies have been described in the scientific literature in which one or two CDRs can be dispensed with for binding. Padlan et al. (FASEB J. 1995; 9(1):133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vaj dos et al., J Mol Biol. 2002; 320(2):415-428).

CDR residues not contacting antigen can be identified based on previous studies (for example residues H60-H65 in V_{H}-CDR2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically. Empirical substitutions can be conservative or non-conservative substitutions.

The anti-PD-1/anti-4-1BB bispecific binding proteins disclosed herein (or any of their individual constituents) may comprise one or more amino acid substitutions, insertions and/or deletions in the framework and/or CDR regions of the heavy and/or light chain variable domains as compared to the corresponding germline sequences. Such mutations can be readily ascertained by comparing the amino acid sequences disclosed herein to germline sequences available from, for example, public antibody sequence databases. The present disclosure includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present disclosure may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure.

The present disclosure also includes anti-PD-1/anti-4-1BB bispecific binding proteins (or any of their individual constituents) comprising variants of any of the HCVR, LCVR, and/or CDR amino acid sequences disclosed herein having one or more conservative substitutions. For example, the present disclosure includes anti-PD-1 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein; or anti-4-1BB immunoglobulin single variable domains having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “human antibody”, as used herein, is intended to include antibodies having variable and constant regions derived from human germline immunoglobulin sequences. The human mAbs of the disclosure may include amino acid residues not encoded by human germline immunoglobulin sequences (e.g., mutations introduced by random or site-specific mutagenesis in vitro or by somatic mutation in vivo), for example in the CDRs and in particular in CDR3. However, the term “human antibody”, as used herein, is not intended to include mAbs in which CDR sequences derived from the germline of another mammalian species (e.g., mouse), have been grafted onto human FR sequences. The term includes antibodies recombinantly produced in a non-human mammal, or in cells of a non-human mammal. The term is not intended to include antibodies isolated from or generated in a human subject.

The term “recombinant”, as used herein, refers to antibodies or antigen-binding fragments thereof of the disclosure created, expressed, isolated or otherwise obtained by technologies or methods known in the art as recombinant DNA technology which include, e.g., DNA splicing and transgenic expression. The term refers to antibodies or antigen-binding fragments thereof expressed in a non-human mammal (including transgenic non-human mammals, e.g., transgenic mice), or a cell (e.g., CHO cells) expression system or isolated from a recombinant combinatorial human antibody library.

The term “multispecific antigen-binding molecules”, as used herein, refers to bispecific, tri-specific or multi-specific antigen-binding molecules, and antigen-binding fragments thereof. Multispecific antigen-binding molecules typically contain antigen-binding domains specific for more than one antigen. For antigen-binding molecules binding to more than one epitope of a same antigen, the term “multiparatopic” (e.g., biparatopic, triparatopic, and the like) is preferred, although the literature is inconsistent in this regard and authors may use the term multispecific to refer to antigen-binding molecules binding to more than one epitope of a same antigen. The context in which these terms are employed will clear the disambiguation.

A multispecific antigen-binding molecule can be a single multifunctional polypeptide, or it can be a multimeric complex of two or more polypeptides that are covalently or non-covalently associated with one another. The term “multispecific antigen-binding molecules” includes antibodies or antigen-binding fragment thereof of the present disclosure that may be linked to or co-expressed with another functional molecule, e.g., another peptide or protein. For example, an antibody or antigen-binding fragment thereof can be functionally linked (e.g., by chemical coupling, genetic fusion, non-covalent association or otherwise) to one or more other molecular entities, such as a protein or fragment thereof to produce a bi-specific or a multi-specific antigen-binding molecule with a second binding specificity. According to the present disclosure, the term “multispecific antigen-binding molecules” also includes bispecific, trispecific or multispecific antibodies or antigen-binding fragments thereof. In certain exemplary embodiments, an antibody or antigen-binding fragment thereof of the present disclosure is functionally linked to another antibody or antigen-binding fragment thereof to produce a bispecific antibody with a second binding specificity.

In exemplary embodiments, the antibodies of the present disclosure are bispecific antibodies. Bispecific antibodies can be monoclonal, e.g., human or humanized, antibodies that have binding specificities for at least two different antigens. In an exemplary embodiment, the bispecific antibody, fragment thereof and so on has binding specificities directed towards PD-1 and 4-1BB.

Methods for making bispecific antibodies are well-known. Traditionally, the recombinant production of bispecific antibodies is based on the co-expression of two immunoglobulin heavy chain/light chain pairs, where the two heavy chains have different specificities (Milstein et al., Nature. 1983 Oct. 6-12;305(5934):537-40). Because of the random assortment of immunoglobulin heavy and light chains, the hybridomas (quadromas) produce a potential mixture of ten different antibody molecules, of which only one has the correct bispecific structure. The purification of the correct molecule is usually accomplished by affinity chromatography steps. Similar procedures are disclosed in WO 1993/08829 and in Traunecker et al. (EMBO J. 1991 December; 10(12):3655-9). Other methods for making bispecific antibodies are provided in, for example, Kufer et al., Trends Biotechnol. 2004 May; 22(5):238-44.

Antibody variable domains with the desired binding specificities can be fused to immunoglobulin constant domain sequences. The fusion typically is with an immunoglobulin heavy chain constant domain, comprising at least part of the hinge, C_{H}2, and C_{H}3 regions. It may have the first heavy chain constant region (C_{H}1) containing the site necessary for light chain binding present in at least one of the fusions. DNAs encoding the immunoglobulin heavy chain fusions and, if desired, the immunoglobulin light chain, are inserted into separate expression vectors, and are co-transformed into a suitable host organism. For further details of generating bispecific antibodies see, for example, Suresh et al., Methods Enzymol. 1986; 121:210-28.

The term “specifically bind(s)”, “bind(s) specifically to”, and any declension thereof, means that an antibody or antigen-binding fragment thereof forms a complex with an antigen that is relatively stable under physiologic conditions. Specific binding can be characterized by an equilibrium dissociation constant (denoted “K_{D}”) of at least about 1×10⁻⁸ M or less (e.g., a smaller K_{D} denotes a tighter binding). Methods for determining whether two molecules specifically bind are well-known in the art and include, for example, equilibrium dialysis, surface plasmon resonance, and the like. Moreover, multi-specific antibodies that bind to one domain in PD-1 and one or more additional antigens or a bi-specific that binds to two different regions of PD-1 are nonetheless considered antibodies that “specifically bind”, as used herein.

The term “high affinity”, with respect to an antibody or antigen-binding fragment thereof, refers to those mAbs having a binding affinity to antigen, e.g., PD-1 and/or 4-1BB, expressed as K_{D}, of at least 10⁻⁷ M; at least 10⁻⁸ M; at least 10⁻⁹ M, at least 10⁻¹⁰ M, or at least 10⁻¹¹ M, as measured by surface plasmon resonance, e.g., BIACORE™ or solution-affinity ELISA.

The term “off rate” or “K_{off}” refers a constant used to characterize how quickly an antibody or antigen-binding fragment thereof dissociates from its antigen, e.g., PD-1 and/or 4-1BB. By “slow off rate”, it is meant an antibody or antigen-binding fragment thereof that dissociates from antigen, e.g., PD-1 and/or 4-1BB, with a rate constant of 1×10⁻³ s-1 or less, or of 1×10⁻⁴ s⁻¹ or less, as determined by surface plasmon resonance, e.g., BIACORE™.

The term “surface plasmon resonance”, as used herein, refers to an optical phenomenon that allows for the analysis of real-time biomolecular interactions by detection of alterations in protein concentrations within a biosensor matrix, for example using the BIACORE™ system (Pharmacia Biosensor AB, Uppsala, Sweden and Piscataway, N.J.).

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment”, as used herein, refers to one or more fragments of an antibody that retain the ability to bind to PD-1 and/or 4-1BB. In specific embodiments, antibody or antibody fragments of the disclosure may be conjugated to a moiety such a ligand or a therapeutic moiety (“immunoconjugate”), such as an antibiotic, a second anti-PD-1 and/or anti-4-1BB antibody, or an antibody to another antigen such a tumor-specific antigen, a virally-infected cell antigen, a Fc receptor, a T-cell receptor, or a T-cell co-inhibitor, or an immunotoxin, or any other therapeutic moiety useful for treating a disease or condition including cancer, or chronic viral infection.

An “isolated antibody”, as used herein, is intended to refer to an antibody that is substantially free of other antibodies (Abs) having different antigenic specificities (e.g., an isolated antibody that specifically binds PD-1 and/or 4-1BB, or an antigen-binding fragment thereof, is substantially free of Abs that specifically bind antigens other than PD-1 and/or 4-1BB).

A “blocking antibody”, a “neutralizing antibody”, or an “antagonist antibody”, as used herein, is intended to refer to an antibody whose binding to its target, e.g., PD-1, results in inhibition of at least one biological activity of that target, e.g., PD-1. For example, an antibody or antigen-binding fragment thereof of the disclosure may prevent or block a ligand such as PD-L1 binding to PD-1.

An “activating antibody”, an “enhancing antibody”, or an “agonist antibody”, as used herein, is intended to refer to an antibody whose binding to its target, e.g., 4-1BB, results in increasing or stimulating at least one biological activity of that target, e.g., 4-1BB. For example, an antibody or antigen-binding fragment thereof of the disclosure may mimic the action of a target's natural ligand, e.g., 4-1BBL to promote TRAFs-mediated activation of NF-κB and MAPK intracellular signaling cascades, ultimately resulting in cell activation, proliferation and survival.

In certain exemplary embodiments, an antibody or antigen-binding fragment thereof of the disclosure is both a blocking antibody and an activating antibody. For example, an anti-PD-1/anti-4-1BB bispecific binding protein of the disclosure may act as an antagonist anti-PD-1 binding protein and as an agonist anti-4-1BB binding protein.

The term “pure agonist”, as used herein, is intended to refer to an activating antibody or antigen-binding fragment thereof as defined hereinabove, whose binding to its antigen results in increasing or stimulating at least one biological activity of the antigen (by activating a T-cell via 4-1BB signaling) (i) in soluble conditions and/or (ii) in the absence of a cross-linking reagent and/or (iii) in an FcγR-independent manner (i.e., independently of Fcγ receptor engagement) and/or (iv) in the absence of target-mediated crosslinking of the antigen.

The term “Fc-mediated crosslinking”, as used herein, refers to the crosslinking of a protein comprising an Fc domain (e.g., an antibody) via binding of said Fc domain to an Fc-binding moiety, e.g., an anti-Fc antibody or an Fc receptor.

As used herein, the term “Fc receptor” refers to the surface receptor protein found on immune cells including B-lymphocytes, natural killer cells, macrophages, basophils, neutrophils, and mast cells, which has a binding specificity for the Fc region of an antibody. The term “Fc receptor” includes, but is not limited to, a Fcγ receptor [e.g., FcγRI (CD64), FcγRIIA (CD32), FcγRIIB (CD32), FcγRIIIA (CD16a), and FcγRIIIB (CD16b)], Fcα receptor (e.g., FcαRI or CD89) and Fcε receptor [e.g., FcεRI, and FcεRII (CD23)].

The term “target-mediated crosslinking”, as used herein, refers to the crosslinking of an antibody or antigen-binding fragment thereof, via interaction of this antibody or antigen-binding fragment thereof with, e.g., a tumor-associated antigen (TAA), an immune cell surface marker, a stromal antigen or any other target expressed in cis or trans by a tumor cell, an immune cell, and/or a normal cell.

The term “epitope” refers to an antigenic determinant that interacts with a specific antigen binding site in the variable region of an antibody known as a “paratope”. A single antigen may have more than one epitope. Thus, different antibodies may bind to different areas on a same antigen and may have different biological effects. The term “epitope” also refers to a site on an antigen to which B- and/or T-cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The term “substantial identity” or “substantially identical”, when referring to a nucleic acid or fragment thereof, indicates that, when optimally aligned with appropriate nucleotide insertions or deletions with another nucleic acid (or its complementary strand), there is nucleotide sequence identity in at least about 90%, or at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or a substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 90% sequence identity, or at least 95%, 96%, 97%, 98% or 99% of sequence identity. In exemplary embodiments, residue positions, which are not identical, may differ by conservative amino acid substitutions. A “conservative amino acid substitution” is one in which an amino acid residue is substituted by another amino acid residue having a side chain (R group) with similar chemical properties (e.g., in terms of charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (Methods Mol Biol. 1994; 24:307-31), which is herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Exemplary conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (Science. 1992 Jun. 5; 256(5062):1443-5), herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild-type protein and a mutein thereof. See, e.g., GCG Version 6.1.

Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson, Methods Mol Biol. 2000; 132:185-219). Another exemplary algorithm when comparing a sequence of the disclosure to a database containing a large number of sequences from different organisms is the computer program BLAST, especially BLASTP or TBLASTN, using default parameters. See, e.g., Altschul et al., J Mol Biol. 1990 Oct. 5; 215(3):403-10 and Altschul et al., Nucleic Acids Res. 1997 Sep. 1; 25(17):3389-402, each of which is herein incorporated by reference.

In some embodiments, the antibody or antigen-binding fragment thereof disclosed herein may be “conditionally-active”. By “conditionally-active”, it is meant herein that the antibody or antigen-binding fragment thereof is capable of binding to its antigen (i.e., to be active) only under certain condition. In some exemplary embodiments, a conditionally-active antibody or antigen-binding fragment thereof comprises a masking moiety.

As used herein, the terms “mask”, “masking domain” or “masking moiety” refer to a moiety that is added to an antibody or antigen-binding fragment thereof to reduce the ability of the antibody to bind its antigen. The mask serves to prevent or reduce antigen binding by one or more CDR sequences of the antibody or antigen-binding fragment thereof. Masking moieties include, but are not limited to, autologous hinge domains, coiled-coil domains, non-antibody protein fragments, antibody fragments, affinity peptides, cross-masking antibodies, bivalent peptide-double strand DNA conjugates and the like. For a review of suitable antibody masking moieties, see Lin et al. (J Biomed Sci. 2020 Jun. 25; 27(1):76).

In certain exemplary embodiments, a masking moiety is a polypeptide that may be removed from the antibody or antigen-binding fragment thereof by cleavage of a cleavable linker connecting the masking moiety to the antibody or antigen-binding fragment thereof, thereby allowing the antibody or antigen-binding fragment thereof to bind to its target antigen. In particularly exemplary embodiments, a masking domain of an antibody or antigen-binding fragment thereof is cleaved at a tumor site, e.g., at a tumor bed or at a lymph node. The cleavable linker may a protease-cleavable linker. In certain exemplary embodiments, the cleavable linker comprises at least one substrate for a tumor-specific protease.

By the phrase “therapeutically effective amount” is meant an amount that produces the desired effect for which it is administered. The exact amount will depend on the purpose of the treatment, and will be ascertainable by one skilled in the art using known techniques (see, for example, Lloyd (1999) The Art, Science and Technology of Pharmaceutical Compounding).

The term “subject”, as used herein, refers to an animal, e.g., a mammal, in need of amelioration, prevention and/or treatment of a disease or disorder such as cancer, or a chronic viral infection. In some embodiments, the subject is a human subject in need of amelioration, prevention and/or treatment of a disease or disorder such as cancer, or a chronic viral infection.

As used herein, “anti-cancer drug” means any agent useful to treat cancer including, but not limited to, cytotoxins and agents such as antimetabolites, alkylating agents, anthracyclines, antibiotics, antimitotic agents, procarbazine, hydroxyurea, asparaginase, corticosteroids, mytotane (O,P′-(DDD)), biologics (e.g., antibodies and interferons) and radioactive agents. As used herein, “a cytotoxin or cytotoxic agent”, also refers to a chemotherapeutic agent and means any agent that is detrimental to cells. Examples include TAXOL® (paclitaxel), temozolamide, cytochalasin B, gramicidin D, ethidium bromide, emetine, cisplatin, mitomycin, etoposide, tenoposide, vincristine, vinbiastine, coichicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

As used herein, the term “antiviral drug” refers to any drug or therapy used to treat, prevent, or ameliorate a viral infection in a host subject. The term “antiviral drug” includes, but is not limited to zidovudine, lamivudine, abacavir, ribavirin, lopinavir, efavirenz, cobicistat, tenofovir, rilpivirine, analgesics and corticosteroids. In the context of the present disclosure, the viral infections include long-term or chronic infections caused by viruses including, but not limited to, human immunodeficiency virus (HIV), hepatitis B virus (HBV), hepatitis C virus (HCV), human papilloma virus (HPV), lymphocytic choriomeningitis virus (LCMV), and simian immunodeficiency virus (SIV).

The antibodies and antigen-binding fragments of the subject disclosure specifically bind to PD-1 and modulate the interaction of PD-1 with a ligand such as, e.g., PD-L1; and specifically bind 4-1BB and modulate the interaction of 4-1BB with a ligand such as, e.g., 4-1BBL. The anti-PD-1/anti-4-1BB antibodies may bind to PD-1 and 4-1BB with high affinity or with low affinity. In certain embodiments, the antibodies of the present disclosure may bind to PD-1 and block the interaction of PD-1 with PD-L1 (i.e., they are PD-1 antagonists), and may bind to 4-1BB and stimulate or enhance T-cell activation (i.e., they are 4-1BB agonists). In some embodiments, the antibodies may be useful for stimulating or enhancing the immune response and/or for treating a subject suffering from cancer, or a chronic viral infection. The antibodies when administered to a subject in need thereof may reduce the chronic infection by a virus such as HIV, LCMV or HBV in the subject. They may be used to inhibit the growth of tumor cells in a subject. They may be used alone or as adjunct therapy with other therapeutic moieties or modalities known in the art for treating cancer, or viral infection.

### DETAILED DESCRIPTION OF THE INVENTION

An object of the present invention relates to an immunoglobulin single variable domain (ISV) specifically binding to 4-1BB, wherein the ISV has pure agonist activity.

In some embodiments, pure agonist activity means that the ISV is capable of activating a T-cell via 4-1BB signaling, (i) in soluble conditions, and/or (ii) in the absence of a cross-linking reagent, and/or (iii) in an FcγR-independent manner (i.e., independently of Fcγ receptor engagement), and/or (iv) in the absence of target-mediated crosslinking of 4-1BB. In some embodiments, pure agonist activity is determined by NF-κB pathway activation assay in the absence of a cross-linking reagent. An example of NF-κB pathway activation assay to determine pure agonist activity of an ISV is described in Example 1 herein; the skilled person is also aware of other suitable NF-κB pathway activation assays which have already been extensively described in the literature.

In some embodiments, the ISV competes with 4-1BBL for 4-1BB binding.

In some embodiments, 4-1BB is human 4-1BB, an exemplary amino acid sequence of which is shown in SEQ ID NO: 13.

In some embodiments, the ISV cross-reacts with cynomolgus 4-1BB, i.e., the ISV specifically binds to human 4-1BB and also to cynomolgus 4-1BB.

In some embodiments, the ISV interacts with the cysteine-rich domain 2 (CRD2) and/or cysteine-rich domain 3 (CRD3) domain of 4-1BB; preferably the ISV interacts with the CRD2 and CRD3 domains of 4-1BB. In some embodiments, the ISV interacts with at least a first patch of amino acid residues from the CRD2 domain of 4-1BB comprising amino acid residues K69, G70, V71, F72 and R73, and with at least a second and third patch of amino acid residues from the CRD3 domain of 4-1BB comprising amino acid residues S100, M101, C102, E103 and Q104, and amino acid residues K114, K115, and G116, respectively (numbering based on SEQ ID NO: 13). In some embodiments, the ISV interacts with at least one amino acid residue of 4-1BB selected from the group consisting of residues K69, G70, V71, F72, R73, F92, L95, S100, M101, C102, E103, Q104, K114, K115 and G116 of SEQ ID NO: 13.

In some embodiments, the ISV comprises three complementary determining regions (CDRs). As shown in the Example section, of all three CDRs, the ISVs with pure agonist activity described herein interact with their target antigen 4-1BB through their CDR3 only.

In some embodiments, the ISV comprises a CDR3 amino acid sequence found in SEQ ID NOs: 2, 3, 58, 59, 60, or 61. In some embodiments, the ISV comprises a CDR3 amino acid sequence found in SEQ ID NOs: 2 or 3.

In some embodiments, CDR3 comprises or consists of the amino acid sequence:
ARGTRYKLST (SEQ ID NO: 14), ARGTRYKMST (SEQ ID NO: 15), or ARGTRYKIFA (SEQ ID NO: 62), according to IMGT numbering; or
GTRYKMST (SEQ ID NO: 63), GTRYKLST (SEQ ID NO: 64), or GTRYKIFA (SEQ ID NO: 65), according to Kabat or Chothia numbering.

In some embodiments, the ISV comprises a CDR1 amino acid sequence found in SEQ ID NOs: 2, 3, 58, 59, 60, or 61. In some embodiments, the ISV comprises a CDR1 amino acid sequence found in SEQ ID NOs: 2 or 3.

In some embodiments, CDR1 comprises or consists of the amino acid sequence:
GFTFSDHT (SEQ ID NO: 16), GFAFRDFT (SEQ ID NO: 66), GDTFSSYA (SEQ ID NO: 67), or GFTFANYR (SEQ ID NO: 68), according to IMGT numbering; or
DHTMT (SEQ ID NO: 69), DFTMS (SEQ ID NO: 70), SYAMG (SEQ ID NO: 71), or NYRMS (SEQ ID NO: 72), according to Kabat numbering; or
GFTFSDH (SEQ ID NO: 73), GFAFRDF (SEQ ID NO: 74), GDTFSSY (SEQ ID NO: 75), or GFTFANY (SEQ ID NO: 76), according to Chothia numbering.

In some embodiments, the ISV comprises a CDR2 amino acid sequence found in SEQ ID NOs: 2, 3, 58, 59, 60, or 61. In some embodiments, the ISV comprises a CDR2 amino acid sequence found in SEQ ID NOs: 2 or 3.

In some embodiments, CDR2 comprises or consists of the amino acid sequence:
ISSGGSRI (SEQ ID NO: 17), INPSGGSQ (SEQ ID NO: 77), or IKKSGNRT (SEQ ID NO: 78), according to IMGT numbering; or
SISSGGSRIIYADSVKG (SEQ ID NO: 79), SINPSGGSQSYLPSVKG (SEQ ID NO: 80), SINPSGGSQSYHPSVKD (SEQ ID NO: 81), or SIKKSGNRTTYSDSVKG (SEQ ID NO: 82), according to Kabat numbering; or
SSGGSR (SEQ ID NO: 83), NPSGGS (SEQ ID NO: 84), or KKSGNR (SEQ ID NO: 85), according to Chothia numbering.

In some embodiments, the ISV comprises three CDR amino acid sequences found in SEQ ID NOs: 2, 3, 58, 59, 60, or 61. In some embodiments, the ISV comprises three CDR amino acid sequences found in SEQ ID NOs: 2 or 3.

In some embodiments, the ISV comprises three CDRs, wherein:
CDR1 comprises or consists of the amino acid sequence GFTFSDHT (SEQ ID NO: 16), GFAFRDFT (SEQ ID NO: 66), GDTFSSYA (SEQ ID NO: 67), or GFTFANYR (SEQ ID NO: 68);
CDR2 comprises or consists of the amino acid sequence ISSGGSRI (SEQ ID NO: 17), INPSGGSQ (SEQ ID NO: 77), or IKKSGNRT (SEQ ID NO: 78); and
CDR3 comprises or consists of the amino acid sequence ARGTRYKLST (SEQ ID NO: 14), ARGTRYKMST (SEQ ID NO: 15), or ARGTRYKIFA (SEQ ID NO: 62).

In some embodiments, the ISV comprises three CDRs, wherein:
CDR1 comprises or consists of the amino acid sequence DHTMT (SEQ ID NO: 69), DFTMS (SEQ ID NO: 70), SYAMG (SEQ ID NO: 71), or NYRMS (SEQ ID NO: 72);
CDR2 comprises or consists of the amino acid sequence SISSGGSRIIYADSVKG (SEQ ID NO: 79), SINPSGGSQSYLPSVKG (SEQ ID NO: 80), SINPSGGSQSYHPSVKD (SEQ ID NO: 81), or SIKKSGNRTTYSDSVKG (SEQ ID NO: 82); and
CDR3 comprises or consists of the amino acid sequence GTRYKMST (SEQ ID NO: 63), GTRYKLST (SEQ ID NO: 64), or GTRYKIFA (SEQ ID NO: 65).

In some embodiments, the ISV comprises three CDRs, wherein:
CDR1 comprises or consists of the amino acid sequence GFTFSDH (SEQ ID NO: 73), GFAFRDF (SEQ ID NO: 74), GDTFSSY (SEQ ID NO: 75), or GFTFANY (SEQ ID NO: 76);
CDR2 comprises or consists of the amino acid sequence SSGGSR (SEQ ID NO: 83), NPSGGS (SEQ ID NO: 84), or KKSGNR (SEQ ID NO: 85); and
CDR3 comprises or consists of the amino acid sequence GTRYKMST (SEQ ID NO: 63), GTRYKLST (SEQ ID NO: 64), or GTRYKIFA (SEQ ID NO: 65).

In some embodiments, the ISV comprises three CDRs, wherein:
CDR1 comprises or consists of the amino acid sequence GFTFSDHT (SEQ ID NO: 16),
CDR2 comprises or consists of the amino acid sequence ISSGGSRI (SEQ ID NO: 17), and
CDR3 comprises or consists of the amino acid sequence ARGTRYKMST (SEQ ID NO: 15); or
CDR1 comprises or consists of the amino acid sequence DHTMT (SEQ ID NO: 69),
CDR2 comprises or consists of the amino acid sequence SISSGGSRIIYADSVKG (SEQ ID NO: 79), and
CDR3 comprises or consists of the amino acid sequence GTRYKMST (SEQ ID NO: 63); or
CDR1 comprises or consists of the amino acid sequence GFTFSDH (SEQ ID NO: 73),
CDR2 comprises or consists of the amino acid sequence SSGGSR (SEQ ID NO: 83), and
CDR3 comprises or consists of the amino acid sequence GTRYKMST (SEQ ID NO: 63).

In some embodiments, the ISV comprises three CDRs, wherein:
CDR1 comprises or consists of the amino acid sequence GFTFSDHT (SEQ ID NO: 16),
CDR2 comprises or consists of the amino acid sequence ISSGGSRI (SEQ ID NO: 17), and
CDR3 comprises or consists of the amino acid sequence ARGTRYKLST (SEQ ID NO: 14); or
CDR1 comprises or consists of the amino acid sequence DHTMT (SEQ ID NO: 69),
CDR2 comprises or consists of the amino acid sequence SISSGGSRIIYADSVKG (SEQ ID NO: 79), and
CDR3 comprises or consists of the amino acid sequence GTRYKLST (SEQ ID NO: 64); or
CDR1 comprises or consists of the amino acid sequence GFTFSDH (SEQ ID NO: 73),
CDR2 comprises or consists of the amino acid sequence SSGGSR (SEQ ID NO: 83), and
CDR3 comprises or consists of the amino acid sequence GTRYKLST (SEQ ID NO: 64).

In some embodiments, the ISV comprises three CDRs, wherein:
CDR1 comprises or consists of the amino acid sequence GFAFRDFT (SEQ ID NO: 66),
CDR2 comprises or consists of the amino acid sequence INPSGGSQ (SEQ ID NO: 77), and
CDR3 comprises or consists of the amino acid sequence ARGTRYKMST (SEQ ID NO: 15); or
CDR1 comprises or consists of the amino acid sequence DFTMS (SEQ ID NO: 70),
CDR2 comprises or consists of the amino acid sequence SINPSGGSQSYLPSVKG (SEQ ID NO: 80), and
CDR3 comprises or consists of the amino acid sequence GTRYKMST (SEQ ID NO: 63); or
CDR1 comprises or consists of the amino acid sequence GFAFRDF (SEQ ID NO: 74),
CDR2 comprises or consists of the amino acid sequence NPSGGS (SEQ ID NO: 84), and
CDR3 comprises or consists of the amino acid sequence GTRYKMST (SEQ ID NO: 63).

In some embodiments, the ISV comprises three CDRs, wherein:
CDR1 comprises or consists of the amino acid sequence GDTFSSYA (SEQ ID NO: 67);
CDR2 comprises or consists of the amino acid sequence INPSGGSQ (SEQ ID NO: 77); and
CDR3 comprises or consists of the amino acid sequence ARGTRYKIFA (SEQ ID NO: 62); or
CDR1 comprises or consists of the amino acid sequence SYAMG (SEQ ID NO: 71),
CDR2 comprises or consists of the amino acid sequence SINPSGGSQSYHPSVKD (SEQ ID NO: 81), and
CDR3 comprises or consists of the amino acid sequence GTRYKIFA (SEQ ID NO: 65); or
CDR1 comprises or consists of the amino acid sequence GDTFSSY (SEQ ID NO: 75),
CDR2 comprises or consists of the amino acid sequence NPSGGS (SEQ ID NO: 84), and
CDR3 comprises or consists of the amino acid sequence GTRYKIFA (SEQ ID NO: 65).

In some embodiments, the ISV comprises three CDRs, wherein:
CDR1 comprises or consists of the amino acid sequence GFTFANYR (SEQ ID NO: 68);
CDR2 comprises or consists of the amino acid sequence IKKSGNRT (SEQ ID NO: 78); and
CDR3 comprises or consists of the amino acid sequence ARGTRYKMST (SEQ ID NO: 15); or
CDR1 comprises or consists of the amino acid sequence NYRMS (SEQ ID NO: 72),
CDR2 comprises or consists of the amino acid sequence SIKKSGNRTTYSDSVKG (SEQ ID NO: 82), and
CDR3 comprises or consists of the amino acid sequence GTRYKMST (SEQ ID NO: 63); or
CDR1 comprises or consists of the amino acid sequence GFTFANY (SEQ ID NO: 76),
CDR2 comprises or consists of the amino acid sequence KKSGNR (SEQ ID NO: 85), and
CDR3 comprises or consists of the amino acid sequence GTRYKMST (SEQ ID NO: 63).

In some embodiments, the ISV comprises or consists of:
an amino acid sequence selected from the group consisting of SEQ ID NOs: 2, 3, 58, 59, 60, and 61; or
an amino acid sequence sharing at least 70%, 75%, 80%, 85%, 90%, 95%, 96%, 97%, 98%, or 99% of sequence identity over the non-CDR regions of SEQ ID NOs: 2, 3, 58, 59, 60, or 61.

In some embodiments, the ISV comprises or consists of:
an amino acid sequence selected from the group consisting of SEQ ID NOs: 2 and 3; or
an amino acid sequence sharing at least 70%, 75%, 80%, 85%, 90%, 95%, 96%, 97%, 98%, or 99% of sequence identity over the non-CDR regions of SEQ ID NOs: 2 or 3.

In some embodiments, the ISV comprises or consists of an amino acid sequence of SEQ ID NO: 3. In some embodiments, the ISV comprises or consists of an amino acid sequence of SEQ ID NO: 2.

In some embodiments, the ISV is a V_{HH}.

In some embodiments, the ISV is the anti-4-1BB clone #5 V_{HH}, as described herein in the Example section, or a derivative thereof, such as clone #5.1, clone #5a, clone #5b, clone #5c or clone #5d, also described in the Example section.

Another object of the present invention relates to a bivalent or bispecific antigen-binding protein, comprising at least one immunoglobulin single variable domain (ISV) specifically binding to 4-1BB and having pure agonist activity, as described hereinabove.

In some embodiment, the bivalent or bispecific antigen-binding protein comprises at least a second ISV specifically binding to the same target antigen (i.e., 4-1BB), or to another target antigen.

When the at least second ISV specifically binds to another target antigen, said target antigen may be a T-cell antigen, a tumor-associated or tumor-specific antigen, or a non-self antigen, or any other antigen deemed suitable by a person skilled in the art.

When the at least second ISV specifically binds to the same target antigen (i.e., 4-1BB), said at least second ISV may be the same ISV as the at least first ISV with pure agonist activity; or another ISV specifically binding to 4-1BB and having pure agonist activity, including but not limited to, an ISV as described hereinabove; or another ISV specifically binding to 4-1BB but without pure agonist activity. For instance, ISVs specifically binding to 4-1BB but without pure agonist activity are described hereinafter.

Another object of the present invention relates to an immunoglobulin single variable domain (ISV) specifically binding to 4-1BB. In some embodiments, the ISV does not have pure agonist activity.

In some embodiments, the ISV does not compete with 4-1BBL for 4-1BB binding.

In some embodiments, 4-1BB is human 4-1BB, an exemplary amino acid sequence of which is shown in SEQ ID NO: 13.

In some embodiments, the ISV does not, or does substantially not, cross-react with cynomolgus 4-1BB, i.e., the ISV specifically binds to human 4-1BB but not, or substantially not, to cynomolgus 4-1BB.

In some embodiments, the ISV comprises three complementary determining regions (CDRs).

In some embodiments, the ISV comprises a CDR1 amino acid sequence found in SEQ ID NOs: 1 or 4.

In some embodiments, CDR1 comprises or consists of the amino acid sequence GGLFSINT (SEQ ID NO: 86; according to IMGT numbering); or INTGG (SEQ ID NO: 87; according to Kabat numbering); or GGLFSIN (SEQ ID NO: 88; according to Chothia numbering).

In some embodiments, the ISV comprises a CDR2 amino acid sequence found in SEQ ID NOs: 1 or 4.

In some embodiments, CDR2 comprises or consists of the amino acid sequence ITHDDRT (SEQ ID NO: 89; according to IMGT numbering); or TITHDDRTNYAESVKG (SEQ ID NO: 90; according to Kabat numbering); or THDDR (SEQ ID NO: 91; according to Chothia numbering).

In some embodiments, the ISV comprises a CDR3 amino acid sequence found in SEQ ID NOs: 1 or 4.

In some embodiments, CDR3 comprises or consists of the amino acid sequence RLGSAAIRGY (SEQ ID NO: 92; according to IMGT numbering); or GSAAIRGY (SEQ ID NO: 93; according to Kabat or Chothia numbering).

In some embodiments, the ISV comprises three CDR amino acid sequences found in SEQ ID NOs: 1 or 4.

In some embodiments, the ISV comprises three CDRs, wherein:
CDR1 comprises or consists of the amino acid sequence GGLFSINT (SEQ ID NO: 86);
CDR2 comprises or consists of the amino acid sequence ITHDDRT (SEQ ID NO: 89); and
CDR3 comprises or consists of the amino acid sequence RLGSAAIRGY (SEQ ID NO: 92).

In some embodiments, the ISV comprises three CDRs, wherein:
CDR1 comprises or consists of the amino acid sequence INTGG (SEQ ID NO: 87);
CDR2 comprises or consists of the amino acid sequence TITHDDRTNYAESVKG (SEQ ID NO: 90); and
CDR3 comprises or consists of the amino acid sequence GSAAIRGY (SEQ ID NO: 93).

In some embodiments, the ISV comprises three CDRs, wherein:
CDR1 comprises or consists of the amino acid sequence GGLFSIN (SEQ ID NO: 88);
CDR2 comprises or consists of the amino acid sequence THDDR (SEQ ID NO: 91); and
CDR3 comprises or consists of the amino acid sequence GSAAIRGY (SEQ ID NO: 93).

In some embodiments, the ISV comprises or consists of:
an amino acid sequence selected from the group consisting of SEQ ID NOs: 1 and 4, or
an amino acid sequence sharing at least 70%, 75%, 80%, 85%, 90%, 95%, 96%, 97%, 98%, or 99% of sequence identity over the non-CDR regions of SEQ ID NOs: 1 or 4.

In some embodiments, the ISV comprises or consists of an amino acid sequence with SEQ ID NO: 4. In some embodiments, the ISV comprises or consists of an amino acid sequence with SEQ ID NO: 1.

In some embodiments, the ISV is a V_{HH}.

In some embodiments, the ISV is the anti-4-1BB clone #2 V_{HH}, as described herein in the Example section, or a derivative thereof, such as clone #2.1, also described in the Example section.

In some embodiments, the ISV further comprises at least one masking moiety. In some embodiments, the masking moiety reduces or inhibits the binding of the ISV to its target antigen 4-1BB.

In some embodiments, the masking moiety comprises or consists of an amino acid sequence CPELQGIFC (SEQ ID NO: 94), or an amino acid sequence sharing at least 70%, 75%, 80%, 85%, 90%, 95%, 96%, 97%, 98%, or 99% of sequence identity with SEQ ID NO: 94.

In some embodiments, the masking moiety comprises or consists of an amino acid sequence CPELQGIFCYR (SEQ ID NO: 95), or an amino acid sequence sharing at least 70%, 75%, 80%, 85%, 90%, 95%, 96%, 97%, 98%, or 99% of sequence identity with SEQ ID NO: 95.

In some embodiments, the masking moiety comprises or consists of an amino acid sequence VEVCPELQGIFC (SEQ ID NO: 96), or an amino acid sequence sharing at least 70%, 75%, 80%, 85%, 90%, 95%, 96%, 97%, 98%, or 99% of sequence identity with SEQ ID NO: 96.

In some embodiments, the masking moiety comprises or consists of an amino acid sequence VEVCPELQGIFCYR (SEQ ID NO: 97), or an amino acid sequence sharing at least 70%, 75%, 80%, 85%, 90%, 95%, 96%, 97%, 98%, or 99% of sequence identity with SEQ ID NO: 97.

In some embodiments, the masking moiety comprises or consists of an amino acid sequence X₁X₂X₃X₄X₅X₆X₇X₈X₉X₁₀X₁₁CPELQGIFCX₁₂X₁₃ (SEQ ID NO: 98), wherein each of X₁-X₁₃ represents any amino acid residue. In some embodiments, the masking moiety comprises or consists of an amino acid sequence X₁X₂X₃X₄X₅X₆X₇X₈X₉X₁₀X₁₁CPELQGIFCYR (SEQ ID NO: 99), wherein each of X₁-X₁₁ represents any amino acid residue. In some embodiments, the masking moiety comprises or consists of an amino acid sequence X₁X₂X₃X₄X₅X₆X₇X₈VEVCPELQGIFCX₁₂X₁₃ (SEQ ID NO: 100), wherein each of X₁-X₅ and of X₁₂-X₁₃ represents any amino acid residue. In some embodiments, the masking moiety comprises or consists of an amino acid sequence X₁X₂X₃X₄X₅X₆X₇X₈VEVCPELQGIFCYR (SEQ ID NO: 101), wherein each of X₁-X₅ represents any amino acid residue.

In some embodiments, the masking moiety comprises or consists of an amino acid sequence EVGSX₅X₆X₇X₈X₉X₁₀X₁₁CPELQGIFCX₁₂X₁₃ (SEQ ID NO: 102), wherein each of X₅-X₁₃ represents any amino acid residue. In some embodiments, the masking moiety comprises or consists of an amino acid sequence EVGSX₅X₆X₇X₈X₉X₁₀X₁₁CPELQGIFCYR (SEQ ID NO: 103), wherein each of X₅-X₁₁ represents any amino acid residue. In some embodiments, the masking moiety comprises or consists of an amino acid sequence EVGSX₅X₆X₇X₈VEVCPELQGIFCX₁₂X₁₃ (SEQ ID NO: 104), wherein each of X₅-X₈ and of X₁₂-X₁₃ represents any amino acid residue. In some embodiments, the masking moiety comprises or consists of an amino acid sequence EVGSX₅X₆X₇X₈VEVCPELQGIFCYR (SEQ ID NO: 105), wherein each of X₅-X₈ represents any amino acid residue.

In some embodiments, the masking moiety comprises or consists of an amino acid sequence of SEQ ID NO: 44 or 45, or an amino acid sequence sharing at least 70%, 75%, 80%, 85%, 90%, 95%, 96%, 97%, 98%, or 99% of sequence identity with SEQ ID NO: 44 or 45.

In some embodiments, the ISV further comprises at least one linker between the masking moiety and the ISV. In some embodiments, the at least one linker is cleavable. In some embodiments, the at least one linker is cleavable by at least one tumor-specific protease. In some embodiments, the at least one tumor-specific protease is selected from the group consisting of matrix metalloproteinase-9 (MMP-9), urokinase-type plasminogen activator (uPa), matrix metalloproteinase-2 (MMP-2), matriptase, legumain, kallikrein-related peptidase-3, human neutrophil elastase, proteinase 3 (Pr3), cathepsin B, and cathepsin K. In some embodiments, the at least one tumor-specific protease is MMP-9 or uPa, or a combination thereof.

In some embodiments, the linker comprises or consists of an amino acid sequence of SEQ ID NO: 56 and/or 57. However, a person skilled in the art can readily appreciate that other amino acid sequences are suitable to serve as a linker cleavable by tumor-specific proteases. Such amino acid sequences are well-known in the art.

In some embodiments, the linker comprises or consists of an amino acid sequence of SEQ ID NO: 46 or 47.

In some embodiments, cleavage of the at least one linker releases the masking moiety and restores the binding of the ISV to its target antigen 4-1BB.

In some embodiments, the masking moiety and cleavable linker may be fused to the N-terminus or C-terminus of the ISV; preferably the masking moiety and cleavable linker are fused to the N-terminus of the ISV.

In some embodiments, the masked ISV (i.e., including the masking moiety and cleavable linker) comprises or consists of the amino acid sequence SEQ ID NOs: 48, 49, 50 or 51, or an amino acid sequence sharing at least 70%, 75%, 80%, 85%, 90%, 95%, 96%, 97%, 98%, or 99% of sequence identity with SEQ ID NOs: 48, 49, 50 or 51; preferably an amino acid sequence sharing at least 70%, 75%, 80%, 85%, 90%, 95%, 96%, 97%, 98%, or 99% of sequence identity over the non-CDR regions of SEQ ID NOs: 48, 49, 50 or 51; more preferably an amino acid sequence sharing at least 70%, 75%, 80%, 85%, 90%, 95%, 96%, 97%, 98%, or 99% of sequence identity over the non-CDR, masking moiety and cleavable linker regions of SEQ ID NOs: 48, 49, 50 or 51.

Another object of the present invention relates to an immunoglobulin single variable domain (ISV) specifically binding to 4-1BB, wherein the ISV comprises at least one masking moiety reducing or inhibiting the binding of the ISV to its target antigen 4-1BB.

In some embodiments, the masking moiety comprises or consists of an amino acid sequence CPELQGIFC (SEQ ID NO: 94), or an amino acid sequence sharing at least 70%, 75%, 80%, 85%, 90%, 95%, 96%, 97%, 98%, or 99% of sequence identity with SEQ ID NO: 94.

In some embodiments, the masking moiety comprises or consists of an amino acid sequence CPELQGIFCYR (SEQ ID NO: 95), or an amino acid sequence sharing at least 70%, 75%, 80%, 85%, 90%, 95%, 96%, 97%, 98%, or 99% of sequence identity with SEQ ID NO: 95.

In some embodiments, the masking moiety comprises or consists of an amino acid sequence VEVCPELQGIFC (SEQ ID NO: 96), or an amino acid sequence sharing at least 70%, 75%, 80%, 85%, 90%, 95%, 96%, 97%, 98%, or 99% of sequence identity with SEQ ID NO: 96.

In some embodiments, the masking moiety comprises or consists of an amino acid sequence VEVCPELQGIFCYR (SEQ ID NO: 97), or an amino acid sequence sharing at least 70%, 75%, 80%, 85%, 90%, 95%, 96%, 97%, 98%, or 99% of sequence identity with SEQ ID NO: 97.

In some embodiments, the masking moiety comprises or consists of an amino acid sequence X₁X₂X₃X₄X₅X₆X₇X₈X₉X₁₀X₁CPELQGIFCX₁₂X₁₃ (SEQ ID NO: 98), wherein each of X₁-X₁₃ represents any amino acid residue. In some embodiments, the masking moiety comprises or consists of an amino acid sequence X₁X₂X₃X₄X₅X₆X₇X₈X₉X₁₀X₁₁CPELQGIFCYR (SEQ ID NO: 99), wherein each of X₁-X₁₁ represents any amino acid residue. In some embodiments, the masking moiety comprises or consists of an amino acid sequence X₁X₂X₃X₄X₅X₆X₇X₈VEVCPELQGIFCX₁₂X₁₃ (SEQ ID NO: 100), wherein each of X₁-X₈ and of X₁₂-X₁₃ represents any amino acid residue. In some embodiments, the masking moiety comprises or consists of an amino acid sequence X₁X₂X₃X₄X₅X₆X₇X₈VEVCPELQGIFCYR (SEQ ID NO: 101), wherein each of X₁-X₈ represents any amino acid residue.

In some embodiments, the masking moiety comprises or consists of an amino acid sequence EVGSX₅X₆X₇X₈X₉X₁₀X₁₁CPELQGIFCX₁₂X₁₃ (SEQ ID NO: 102), wherein each of X₅-X₁₃ represents any amino acid residue. In some embodiments, the masking moiety comprises or consists of an amino acid sequence EVGSX₅X₆X₇X₈X₉X₁₀X₁₁CPELQGIFCYR (SEQ ID NO: 103), wherein each of X₅-X₁₁ represents any amino acid residue. In some embodiments, the masking moiety comprises or consists of an amino acid sequence EVGSX₅X₆X₇X₈VEVCPELQGIFCX₁₂X₁₃ (SEQ ID NO: 104), wherein each of X₅-X₈ and of X₁₂-X₁₃ represents any amino acid residue. In some embodiments, the masking moiety comprises or consists of an amino acid sequence EVGSX₅X₆X₇X₈VEVCPELQGIFCYR (SEQ ID NO: 105), wherein each of X₅-X₈ represents any amino acid residue.

In some embodiments, the masking moiety comprises or consists of an amino acid sequence of SEQ ID NO: 44 or 45, or an amino acid sequence sharing at least 70%, 75%, 80%, 85%, 90%, 95%, 96%, 97%, 98%, or 99% of sequence identity with SEQ ID NO: 44 or 45.

In some embodiments, the ISV further comprises at least one linker between the masking moiety and the ISV. In some embodiments, the at least one linker is cleavable. In some embodiments, the at least one linker is cleavable by at least one tumor-specific protease. In some embodiments, the at least one tumor-specific protease is selected from the group consisting of matrix metalloproteinase-9 (MMP-9), urokinase-type plasminogen activator (uPa), matrix metalloproteinase-2 (MMP-2), matriptase, legumain, kallikrein-related peptidase-3, human neutrophil elastase, proteinase 3 (Pr3), cathepsin B, and cathepsin K. In some embodiments, the at least one tumor-specific protease is MMP-9 or uPa, or a combination thereof.

In some embodiments, the linker comprises or consists of an amino acid sequence of SEQ ID NO: 56 and/or 57. However, a person skilled in the art can readily appreciate that other amino acid sequences are suitable to serve as a linker cleavable by tumor-specific proteases. Such amino acid sequences are well-known in the art.

In some embodiments, the linker comprises or consists of an amino acid sequence of SEQ ID NO: 46 or 47.

In some embodiments, cleavage of the at least one linker releases the masking moiety and restores the binding of the ISV to its target antigen 4-1BB.

In some embodiments, the masking moiety and cleavable linker may be fused to the N-terminus or C-terminus of the ISV; preferably the masking moiety and cleavable linker are fused to the N-terminus of the ISV.

In some embodiments, the ISV is a V_{HH}.

Another object of the present invention relates to an immunoglobulin single variable domain (ISV) specifically binding to a target antigen, wherein the ISV comprises at least one masking moiety reducing or inhibiting the binding to the target antigen.

In some embodiments, the masking moiety comprises or consists of an amino acid sequence CPELQGIFC (SEQ ID NO: 94), or an amino acid sequence sharing at least 70%, 75%, 80%, 85%, 90%, 95%, 96%, 97%, 98%, or 99% of sequence identity with SEQ ID NO: 94.

In some embodiments, the masking moiety comprises or consists of an amino acid sequence CPELQGIFCYR (SEQ ID NO: 95), or an amino acid sequence sharing at least 70%, 75%, 80%, 85%, 90%, 95%, 96%, 97%, 98%, or 99% of sequence identity with SEQ ID NO: 95.

In some embodiments, the masking moiety comprises or consists of an amino acid sequence VEVCPELQGIFC (SEQ ID NO: 96), or an amino acid sequence sharing at least 70%, 75%, 80%, 85%, 90%, 95%, 96%, 97%, 98%, or 99% of sequence identity with SEQ ID NO: 96.

In some embodiments, the masking moiety comprises or consists of an amino acid sequence VEVCPELQGIFCYR (SEQ ID NO: 97), or an amino acid sequence sharing at least 70%, 75%, 80%, 85%, 90%, 95%, 96%, 97%, 98%, or 99% of sequence identity with SEQ ID NO: 97.

In some embodiments, the masking moiety comprises or consists of an amino acid sequence X₁X₂X₃X₄X₅X₆X₇X₈X₉X₁₀X₁₁CPELQGIFCX₁₂X₁₃ (SEQ ID NO: 98), wherein each of X₁-X₁₃ represents any amino acid residue. In some embodiments, the masking moiety comprises or consists of an amino acid sequence X₁X₂X₃X₄X₅X₆X₇X₈X₉X₁₀X₁₁CPELQGIFCYR (SEQ ID NO: 99), wherein each of X₁-X₁₁ represents any amino acid residue. In some embodiments, the masking moiety comprises or consists of an amino acid sequence X₁X₂X₃X₄X₅X₆X₇X₈VEVCPELQGIFCX₁₂X₁₃ (SEQ ID NO: 100), wherein each of X₁-X₈ and of X₁₂-X₁₃ represents any amino acid residue. In some embodiments, the masking moiety comprises or consists of an amino acid sequence X₁X₂X₃X₄X₅X₆X₇X₈VEVCPELQGIFCYR (SEQ ID NO: 101), wherein each of X₁-X₈ represents any amino acid residue.

In some embodiments, the masking moiety comprises or consists of an amino acid sequence EVGSX₅X₆X₇X₈X₉X₁₀X₁₁CPELQGIFCX₁₂X₁₃ (SEQ ID NO: 102), wherein each of X₅-X₁₃ represents any amino acid residue. In some embodiments, the masking moiety comprises or consists of an amino acid sequence EVGSX₅X₆X₇X₈X₉X₁₀X₁₁CPELQGIFCYR (SEQ ID NO: 103), wherein each of X₅-X₁₁ represents any amino acid residue. In some embodiments, the masking moiety comprises or consists of an amino acid sequence EVGSX₅X₆X₇X₈VEVCPELQGIFCX₁₂X₁₃ (SEQ ID NO: 104), wherein each of X₅-X₈ and of X₁₂-X₁₃ represents any amino acid residue. In some embodiments, the masking moiety comprises or consists of an amino acid sequence EVGSX₅X₆X₇X₈VEVCPELQGIFCYR (SEQ ID NO: 105), wherein each of X₅-X₈ represents any amino acid residue.

In some embodiments, the masking moiety comprises or consists of an amino acid sequence of SEQ ID NO: 44 or 45, or an amino acid sequence sharing at least 70%, 75%, 80%, 85%, 90%, 95%, 96%, 97%, 98%, or 99% of sequence identity with SEQ ID NO: 44 or 45.

In some embodiments, the ISV further comprises at least one linker between the masking moiety and the ISV. In some embodiments, the at least one linker is cleavable. In some embodiments, the at least one linker is cleavable by at least one tumor-specific protease. In some embodiments, the at least one tumor-specific protease is selected from the group consisting of matrix metalloproteinase-9 (MMP-9), urokinase-type plasminogen activator (uPa), matrix metalloproteinase-2 (MMP-2), matriptase, legumain, kallikrein-related peptidase-3, human neutrophil elastase, proteinase 3 (Pr3), cathepsin B, and cathepsin K. In some embodiments, the at least one tumor-specific protease is MMP-9 or uPa, or a combination thereof.

In some embodiments, the linker comprises or consists of an amino acid sequence of SEQ ID NO: 56 and/or 57. However, a person skilled in the art can readily appreciate that other amino acid sequences are suitable to serve as a linker cleavable by tumor-specific proteases. Such amino acid sequences are well-known in the art.

In some embodiments, the linker comprises or consists of an amino acid sequence of SEQ ID NO: 46 or 47.

In some embodiments, cleavage of the at least one linker releases the masking moiety and restores the binding of the ISV to its target antigen.

In some embodiments, the masking moiety and cleavable linker may be fused to the N-terminus or C-terminus of the ISV; preferably the masking moiety and cleavable linker are fused to the N-terminus of the ISV.

In some embodiments, the ISV is a V_{HH}.

Another object of the present invention relates to an antibody or antigen-binding fragment thereof that specifically binds to PD-1.

In some embodiments, PD-1 is human PD-1, an exemplary amino acid sequence of which is shown in SEQ ID NO: 42.

In some embodiments, the antibody or antigen-binding fragment thereof has a human germinality index equal to or above 95% for both heavy and light variable regions.

By “human germinality index”, it is meant the proportion of framework residues that are identical between the variable region sequence of the antibody or antigen-binding fragment thereof and the closest related human germline sequence, as coined by Pelat et al. (J Mol Biol. 2008 Dec. 31; 384(5):1400-7).

In some embodiments, the antibody or antigen-binding fragment thereof comprises:
(i) three light chain CDR amino acid sequences found in SEQ ID NO: 7 or 5, and
(ii) three heavy chain CDR amino acid sequences found in SEQ ID NO: 8 or 6.

In some embodiments, the antibody or antigen-binding fragment thereof comprises:
(i) three light chain CDR amino acid sequences found in SEQ ID NO: 7, and
(ii) three heavy chain CDR amino acid sequences found in SEQ ID NO: 8.

In some embodiments, the antibody or antigen-binding fragment thereof comprises:
(i) three light chain CDR amino acid sequences found in SEQ ID NO: 5, and
(ii) three heavy chain CDR amino acid sequences found in SEQ ID NO: 6.

In some embodiments, the antibody or antigen-binding fragment thereof comprises:
(i) a light chain variable region comprising the three following CDR amino acid sequences:
   a. V_{L}-CDR1: QSVPINF (SEQ ID NO: 18) or QSVSINF (SEQ ID NO: 19),
   b. V_{L}-CDR2: EAS, and
   c. V_{L}-CDR3: GQYGSSPYT (SEQ ID NO: 20) or QQYGSSPYT (SEQ ID NO: 21); and
(ii) a heavy chain variable region comprising the three following CDR amino acid sequences:
   a. V_{H}-CDR1: GGSISSSSYF (SEQ ID NO: 22) or GGSISTSSYF (SEQ ID NO: 23),
   b. V_{H}-CDR2: IYRSGST (SEQ ID NO: 24), and
   c. V_{H}-CDR3: ARGITGDPGDY (SEQ ID NO: 25).

In some embodiments, the antibody or antigen-binding fragment thereof comprises:
(i) a light chain variable region comprising the three following CDR amino acid sequences:
   a. V_{L}-CDR1: GASQSVPINFLA (SEQ ID NO: 106) or GASQSVSINFLA (SEQ ID NO: 107),
   b. V_{L}-CDR2: EASSRHT (SEQ ID NO: 108) or EASSRAT (SEQ ID NO: 109), and
   c. V_{L}-CDR3: GQYGSSPYT (SEQ ID NO: 20) or QQYGSSPYT (SEQ ID NO: 21); and
(ii) a heavy chain variable region comprising the three following CDR amino acid sequences:
   a. V_{H}-CDR1: SSSYFWG (SEQ ID NO: 110) or TSSYFWG (SEQ ID NO: 111),
   b. V_{H}-CDR2: SIYRSGSTYYNPSLKS (SEQ ID NO: 112), and
   c. V_{H}-CDR3: GITGDPGDY (SEQ ID NO: 113).

In some embodiments, the antibody or antigen-binding fragment thereof comprises:
(i) a light chain variable region comprising the three following CDR amino acid sequences:
   a. V_{L}-CDR1: GASQSVPINFLA (SEQ ID NO: 106) or GASQSVSINFLA (SEQ ID NO: 107);
   b. V_{L}-CDR2: EASSRHT (SEQ ID NO: 108) or EASSRAT (SEQ ID NO: 109), and
   c. V_{L}-CDR3: GQYGSSPYT (SEQ ID NO: 20) or QQYGSSPYT (SEQ ID NO: 21); and
(ii) a heavy chain variable region comprising the three following CDR amino acid sequences:
   a. V_{H}-CDR1: GGSISSSSY (SEQ ID NO: 114) or GGSISTSSY (SEQ ID NO: 115),
   b. V_{H}-CDR2: YRSGS (SEQ ID NO: 116), and
   c. V_{H}-CDR3: GITGDPGDY (SEQ ID NO: 113).

In some embodiments, the antibody or antigen-binding fragment thereof comprises:
(i) a light chain variable region comprising the three following CDR amino acid sequences:
   a. V_{L}-CDR1: QSVPINF (SEQ ID NO: 18),
   b. V_{L}-CDR2: EAS, and
   c. V_{L}-CDR3: GQYGSSPYT (SEQ ID NO: 20); and
(ii) a heavy chain variable region comprising the three following CDR amino acid sequences:
   a. V_{H}-CDR1: GGSISSSSYF (SEQ ID NO: 22),
   b. V_{H}-CDR2: IYRSGST (SEQ ID NO: 24), and
   c. V_{H}-CDR3: ARGITGDPGDY (SEQ ID NO: 25); or
(i) a light chain variable region comprising the three following CDR amino acid sequences:
   a. V_{L}-CDR1: GASQSVPINFLA (SEQ ID NO: 106),
   b. V_{L}-CDR2: EASSRHT (SEQ ID NO: 108), and
   c. V_{L}-CDR3: GQYGSSPYT (SEQ ID NO: 20); and
(ii) a heavy chain variable region comprising the three following CDR amino acid sequences:
   a. V_{H}-CDR1: SSSYFWG (SEQ ID NO: 110),
   b. V_{H}-CDR2: SIYRSGSTYYNPSLKS (SEQ ID NO: 112), and
   c. V_{H}-CDR3: GITGDPGDY (SEQ ID NO: 113); or
(i) a light chain variable region comprising the three following CDR amino acid sequences:
   a. V_{L}-CDR1: GASQSVPINFLA (SEQ ID NO: 106),
   b. V_{L}-CDR2: EASSRHT (SEQ ID NO: 108), and
   c. V_{L}-CDR3: GQYGSSPYT (SEQ ID NO: 20); and
(ii) a heavy chain variable region comprising the three following CDR amino acid sequences:
   a. V_{H}-CDR1: GGSISSSSY (SEQ ID NO: 114);
   b. V_{H}-CDR2: YRSGS (SEQ ID NO: 116); and
   c. V_{H}-CDR3: GITGDPGDY (SEQ ID NO: 113).

In some embodiments, the antibody or antigen-binding fragment thereof comprises:
(i) a light chain variable region comprising the three following CDR amino acid sequences:
   a. V_{L}-CDR1: QSVSINF (SEQ ID NO: 19),
   b. V_{L}-CDR2: EAS, and
   c. V_{L}-CDR3: QQYGSSPYT (SEQ ID NO: 21); and
(ii) a heavy chain variable region comprising the three following CDR amino acid sequences:
   a. V_{H}-CDR1: GGSISTSSYF (SEQ ID NO: 23), b. V_{H}-CDR2: IYRSGST (SEQ ID NO: 24), and
   c. V_{H}-CDR3: ARGITGDPGDY (SEQ ID NO: 25);
or
(i) a light chain variable region comprising the three following CDR amino acid sequences:
   a. V_{L}-CDR1: GASQSVSINFLA (SEQ ID NO: 107),
   b. V_{L}-CDR2: EASSRAT (SEQ ID NO: 109), and
   c. V_{L}-CDR3: QQYGSSPYT (SEQ ID NO: 21); and
(ii) a heavy chain variable region comprising the three following CDR amino acid sequences:
   a. V_{H}-CDR1: TSSYFWG (SEQ ID NO: 111),
   b. V_{H}-CDR2: SIYRSGSTYYNPSLKS (SEQ ID NO: 112), and
   c. V_{H}-CDR3: GITGDPGDY (SEQ ID NO: 113); or
(i) a light chain variable region comprising the three following CDR amino acid sequences:
   a. V_{L}-CDR1: GASQSVSINFLA (SEQ ID NO: 107),
   b. V_{L}-CDR2: EASSRAT (SEQ ID NO: 109), and
   c. V_{L}-CDR3: QQYGSSPYT (SEQ ID NO: 21); and
(ii) a heavy chain variable region comprising the three following CDR amino acid sequences:
   a. V_{H}-CDR1: GGSISTSSY (SEQ ID NO: 115);
   b. V_{H}-CDR2: YRSGS (SEQ ID NO: 116); and
   c. V_{H}-CDR3: GITGDPGDY (SEQ ID NO: 113).

In some embodiments, the antibody or antigen-binding fragment thereof comprises:
(i) a light chain variable region with SEQ ID NO: 7 or 5, or a light chain variable region sharing at least 70%, 75%, 80%, 85%, 90%, 95%, 96%, 97%, 98%, or 99% of sequence identity over the non-CDR regions of SEQ ID NO: 7 or 5; and
(ii) a heavy chain variable region with SEQ ID NO: 8 or 6, or a heavy chain variable region sharing at least 70%, 75%, 80%, 85%, 90%, 95%, 96%, 97%, 98%, or 99% of sequence identity over the non-CDR regions of SEQ ID NO: 8 or 6.

In some embodiments, the antibody or antigen-binding fragment thereof comprises:
(i) a light chain variable region with SEQ ID NO: 7, or a light chain variable region sharing at least 70%, 75%, 80%, 85%, 90%, 95%, 96%, 97%, 98%, or 99% of sequence identity over the non-CDR regions of SEQ ID NO: 7; and
(ii) a heavy chain variable region with SEQ ID NO: 8, or a heavy chain variable region sharing at least 70%, 75%, 80%, 85%, 90%, 95%, 96%, 97%, 98%, or 99% of sequence identity over the non-CDR regions of SEQ ID NO: 8.

In some embodiments, the antibody or antigen-binding fragment thereof comprises:
(i) a light chain variable region with SEQ ID NO: 5, or a light chain variable region sharing at least 70%, 75%, 80%, 85%, 90%, 95%, 96%, 97%, 98%, or 99% of sequence identity over the non-CDR regions of SEQ ID NO: 5; and
(ii) a heavy chain variable region with SEQ ID NO: 6, or a heavy chain variable region sharing at least 70%, 75%, 80%, 85%, 90%, 95%, 96%, 97%, 98%, or 99% of sequence identity over the non-CDR regions of SEQ ID NO: 6.

In some embodiments, the antibody or antigen-binding fragment thereof comprises a light chain variable region with SEQ ID NO: 7 and a heavy chain variable region with SEQ ID NO: 8.

In some embodiments, the antibody or antigen-binding fragment thereof comprises a light chain variable region with SEQ ID NO: 5 and a heavy chain variable region with SEQ ID NO: 6.

Another object of the present invention relates to a molecule, such as a multispecific antigen-binding protein, comprising at least one immunoglobulin single variable domain (ISV) specifically binding to 4-1BB, wherein the molecule has pure agonist activity, as defined hereinabove.

In some embodiments, the molecule, e.g., the multispecific antigen-binding protein, comprises at least two ISVs specifically binding to 4-1BB. In some embodiments, the at least two ISVs specifically binding to 4-1BB are identical. Alternatively, the at least two ISVs specifically binding to 4-1BB are different. When different, the at least two ISVs specifically binding to 4-1BB may bind to the same epitope, or to overlapping epitopes, or to distinct epitopes of 4-1BB. In some embodiments, the at least two ISVs specifically binding to 4-1BB are different and bind to distinct epitopes of 4-1BB.

In some embodiments, the at least one ISV specifically binding to 4-1BB is an ISV with pure agonist activity as described herein (for instance, but without limitation, an ISV comprising or consisting of an amino acid sequence of SEQ ID NO: 2, 3, 58, 59, 60 or 61). In some embodiments, at least a second ISV specifically binding to 4-1BB is an ISV with pure agonist activity as described herein (for instance, but without limitation, an ISV comprising or consisting of an amino acid sequence of SEQ ID NO: 2, 3, 58, 59, 60 or 61). In some embodiments, at least a second ISV specifically binding to 4-1BB is an ISV without pure agonist activity as described herein (for instance, but without limitation, an ISV comprising or consisting of an amino acid sequence of SEQ ID NO: 1 or 4).

In some embodiments, the molecule, e.g., the multispecific antigen-binding protein, comprises at least two ISVs, wherein one of the at least two ISVs specifically binds to 4-1BB, and the other of the at least two ISVs specifically binds to another target antigen. Said other target antigen may be a T-cell antigen, a tumor-associated or tumor-specific antigen, or a non-self antigen, or any other antigen deemed suitable by a person skilled in the art

In some embodiments, the molecule, e.g., the multispecific antigen-binding protein, comprises at least four ISVs specifically binding to 4-1BB. In some embodiments, the at least four ISVs comprises:
(i) a first set of two identical ISVs specifically binding to 4-1BB, and
(ii) a second set of two other identical ISVs specifically binding to 4-1BB; or
(i′) a first set of two ISVs specifically binding to a first 4-1BB epitope, and
(ii′) a second set of two other ISVs specifically binding to a second 4-1BB epitope.

The first 4-1BB epitope and the second 4-1BB epitope may be identical, overlapping or distinct. In some embodiments, the first 4-1BB epitope and the second 4-1BB epitope are distinct.

In some embodiments, the first set of two ISVs of (i) or (i′) are ISVs with pure agonist activity as described herein (for instance, but without limitation, an ISV comprising or consisting of an amino acid sequence of SEQ ID NO: 2, 3, 58, 59, 60 or 61).

In some embodiments, the second set of two ISVs of (ii) or (ii′) are ISVs without pure agonist activity as described herein (for instance, but without limitation, an ISV comprising or consisting of an amino acid sequence of SEQ ID NO: 1 or 4).

In some embodiments, the molecule, e.g., the multispecific antigen-binding protein, further comprises an antibody Fc region or a fragment thereof. For instance, the Fc region or fragment thereof may be one of an IgG, IgD, IgA, IgM, or IgE Fc region; in particular one of an IgG Fc region, such as an IgG1 or IgG4 Fc region. The Fc region may also be antibody-dependent cytotoxicity (ADCC)-silenced and/or antibody-dependent cellular phagocytosis (ADCP)-silenced. Examples of such silenced Fc regions are known in the art and include, without limitation, IgG1 LALA Fc region, IgG1 NNAS Fc region, and IgG4 P-FALA Fc region. In some embodiment, the Fc region or fragment thereof is one of an IgG1 LALA Fc region.

In some embodiments, the molecule, e.g., the multispecific antigen-binding protein, further comprises at least one Fab fragment.

In some embodiments, the molecule, e.g., the multispecific antigen-binding protein, comprises:
a first polypeptide (i.e., a heavy chain) comprising, preferably from N-terminus to C-terminus:
   a first ISV specifically binding to 4-1BB;
   a second ISV specifically binding to 4-1BB, preferably wherein the second ISV is different from the first ISV;
   at least one C_{H} domain of a Fc region; and
   a variable and constant domain of a Fab fragment;
a second polypeptide (i.e., a light chain) comprising a variable and constant domain of a Fab fragment;
   wherein the variable and constant domains of the first and second polypeptide form a Fab fragment.

In some embodiments, the molecule, e.g., the multispecific antigen-binding protein, further comprises a third and fourth polypeptide identical to the first and second polypeptide, respectively, wherein the at least one C_{H} domain of the first and third polypeptides form an Fc region.

In some embodiments, the variable and constant domain of the first polypeptide are a V_{H} and C_{H}1 domain, and the variable and constant domain of the second polypeptide are a V_{L} and C_{L} domain. Alternatively, the variable and constant domain of the first polypeptide are a V_{L} and C_{L} domain, and the variable and constant domain of the second polypeptide are a V_{H} and C_{H}1 domain.

In some embodiments, the at least one C_{H} domain of the first polypeptide comprise:
C_{H}2 and C_{H}3 domains of IgG;
C_{H}2 and C_{H}3 domains of IgD;
C_{H}2 and C_{H}3 domains of IgA;
C_{H}2, C_{H}3 and C_{H}4 domains of IgM; or
C_{H}2, C_{H}3, and C_{H}4 domains of IgE.

In some embodiments, the at least one C_{H} domain of the first polypeptide comprise C_{H}2 and C_{H}3 domains of IgG.

In some embodiments, the first polypeptide (i.e., the heavy chain) comprises, preferably from N-terminus to C-terminus:
a first ISV specifically binding to 4-1BB;
a first linker;
a second ISV specifically binding to 4-1BB, preferably wherein the second ISV is different from the first ISV;
a second linker;
an IgG hinge region;
an IgG C_{H}2 domain;
and IgG C_{H}3 domain;
a third linker;
a V_{H} domain of a Fab fragment; and
a C_{H}1 domain of a Fab fragment.

In some embodiments, the second polypeptide (i.e., the light chain) comprises, preferably from N-terminus to C-terminus:
a V_{L} domain of a Fab fragment; and
a C_{L} domain of a Fab fragment.

In some embodiments, the at least one Fab fragment binds specifically to a B- and/or T-cell surface protein other than 4-1BB. In some embodiments, the at least one Fab fragment binds specifically to an immune checkpoint molecule. In some embodiments, the at least one Fab fragment is a PD-1 antagonist.

In some embodiments, the at least one Fab fragment is an antigen-binding fragment that specifically binds to PD-1 as described herein (for instance, but without limitation, an antigen-binding fragment comprising or consisting of a light chain variable region with SEQ ID NO: 7 or 5, and a heavy chain variable region with SEQ ID NO: 8 or 6).

In some embodiments, the molecule, e.g., the multispecific antigen-binding protein, comprises at least a first polypeptide with SEQ ID NO: 11 or 9, and at least a second polypeptide with SEQ ID NO: 12 or 10; or at least a first polypeptide comprising an amino acid sequence sharing at least 70%, 75%, 80%, 85%, 90%, 95%, 96%, 97%, 98%, or 99% of sequence identity with SEQ ID NO: 11 or 9, and at least a second polypeptide comprising an amino acid sequence sharing at least 70%, 75%, 80%, 85%, 90%, 95%, 96%, 97%, 98%, or 99% of sequence identity with SEQ ID NO: 12 or 10.

In some embodiments, the molecule, e.g., the multispecific antigen-binding protein, comprises at least a first polypeptide with SEQ ID NO: 11 and at least a second polypeptide with SEQ ID NO: 12.

In some embodiments, the molecule, e.g., the multispecific antigen-binding protein, comprises at least a first polypeptide with SEQ ID NO: 9 and at least a second polypeptide with SEQ ID NO: 10.

In some embodiments, the molecule, e.g., the multispecific antigen-binding protein, is capable of triggering T-cell activation in a TCA and/or MLR assay.

In some embodiments, the molecule, e.g., the multispecific antigen-binding protein, is capable of triggering IFN-γ and/or TNF-α secretion in a CD3-PBMC activation assay.

In some embodiments, the molecule, e.g., the multispecific antigen-binding protein, is capable of reinvigorating exhausted CD8⁺ T-cells in vitro.

In some embodiments, the molecule, e.g., the multispecific antigen-binding protein, is capable of inducing in vitro T_{reg} suppressive activity.

In some embodiments, the molecule, e.g., the multispecific antigen-binding protein, comprises at least one masking moiety. In some embodiments, the masking moiety reduces or inhibits binding of the molecule to at least one of its targets. In some embodiments, the masking moiety reduces or inhibits binding of the molecule to 4-1BB.

In some embodiments, the masking moiety comprises or consists of an amino acid sequence CPELQGIFC (SEQ ID NO: 94), or an amino acid sequence sharing at least 70%, 75%, 80%, 85%, 90%, 95%, 96%, 97%, 98%, or 99% of sequence identity with SEQ ID NO: 94.

In some embodiments, the masking moiety comprises or consists of an amino acid sequence CPELQGIFCYR (SEQ ID NO: 95), or an amino acid sequence sharing at least 70%, 75%, 80%, 85%, 90%, 95%, 96%, 97%, 98%, or 99% of sequence identity with SEQ ID NO: 95.

In some embodiments, the masking moiety comprises or consists of an amino acid sequence VEVCPELQGIFC (SEQ ID NO: 96), or an amino acid sequence sharing at least 70%, 75%, 80%, 85%, 90%, 95%, 96%, 97%, 98%, or 99% of sequence identity with SEQ ID NO: 96.

In some embodiments, the masking moiety comprises or consists of an amino acid sequence VEVCPELQGIFCYR (SEQ ID NO: 97), or an amino acid sequence sharing at least 70%, 75%, 80%, 85%, 90%, 95%, 96%, 97%, 98%, or 99% of sequence identity with SEQ ID NO: 97.

In some embodiments, the masking moiety comprises or consists of an amino acid sequence X₁X₂X₃X₄X₅X₆X₇X₈X₉X₁₀X₁₁CPELQGIFCX₁₂X₁₃ (SEQ ID NO: 98), wherein each of X₁-X₁₃ represents any amino acid residue. In some embodiments, the masking moiety comprises or consists of an amino acid sequence X₁X₂X₃X₄X₅X₆X₇X₈X₉X₁₀X₁CPELQGIFCYR (SEQ ID NO: 99), wherein each of X₁-X₁₁ represents any amino acid residue. In some embodiments, the masking moiety comprises or consists of an amino acid sequence X₁X₂X₃X₄X₅X₆X₇X₈VEVCPELQGIFCX₁₂X₁₃ (SEQ ID NO: 100), wherein each of X₁-X₈ and of X₁₂-X₁₃ represents any amino acid residue. In some embodiments, the masking moiety comprises or consists of an amino acid sequence X₁X₂X₃X₄X₅X₆X₇X₈VEVCPELQGIFCYR (SEQ ID NO: 101), wherein each of X₁-X₈ represents any amino acid residue.

In some embodiments, the masking moiety comprises or consists of an amino acid sequence EVGSX₅X₆X₇X₈X₉X₁₀X₁₁CPELQGIFCX₁₂X₁₃ (SEQ ID NO: 102), wherein each of X₅-X₁₃ represents any amino acid residue. In some embodiments, the masking moiety comprises or consists of an amino acid sequence EVGSX₅X₆X₇X₈X₉X₁₀X₁₁CPELQGIFCYR (SEQ ID NO: 103), wherein each of X₅-X₁₁ represents any amino acid residue. In some embodiments, the masking moiety comprises or consists of an amino acid sequence EVGSX₅X₆X₇X₈VEVCPELQGIFCX₁₂X₁₃ (SEQ ID NO: 104), wherein each of X₅-X₈ and of X₁₂-X₁₃ represents any amino acid residue. In some embodiments, the masking moiety comprises or consists of an amino acid sequence EVGSX₅X₆X₇X₈VEVCPELQGIFCYR (SEQ ID NO: 105), wherein each of X₅-Xx represents any amino acid residue.

In some embodiments, the masking moiety comprises or consists of an amino acid sequence of SEQ ID NO: 44 or 45, or an amino acid sequence sharing at least 70%, 75%, 80%, 85%, 90%, 95%, 96%, 97%, 98%, or 99% of sequence identity with SEQ ID NO: 44 or 45.

In some embodiments, the molecule, e.g., the multispecific antigen-binding protein, further comprises at least one linker between the masking moiety and the molecule. In some embodiments, the at least one linker is cleavable. In some embodiments, the at least one linker is cleavable by at least one tumor-specific protease. In some embodiments, the at least one tumor-specific protease is selected from the group consisting of matrix metalloproteinase-9 (MMP-9), urokinase-type plasminogen activator (uPa), matrix metalloproteinase-2 (MMP-2), matriptase, legumain, kallikrein-related peptidase-3, human neutrophil elastase, proteinase 3 (Pr3), cathepsin B, and cathepsin K. In some embodiments, the at least one tumor-specific protease is MMP-9 or uPa, or a combination thereof.

In some embodiments, the linker comprises or consists of an amino acid sequence of SEQ ID NO: 56 and/or 57. However, a person skilled in the art can readily appreciate that other amino acid sequences are suitable to serve as a linker cleavable by tumor-specific proteases. Such amino acid sequences are well-known in the art.

In some embodiments, the at least one linker comprises or consists of an amino acid sequence of SEQ ID NO: 46 or 47.

In some embodiments, cleavage of the at least one linker releases the masking moiety and restores the binding of the molecule, e.g., the multispecific antigen-binding protein, to at least one of its target antigen. In some embodiments, cleavage of the at least one linker releases the masking moiety and restores the binding of the molecule, e.g., the multispecific antigen-binding protein, to 4-1BB.

In some embodiments, the masking moiety and cleavable linker may be fused to the N-terminus or C-terminus of the heavy chain of the molecule, e.g., of the multispecific antigen-binding protein. Alternatively, the masking moiety and cleavable linker may be fused to the N-terminus or C-terminus of the light chain of the molecule, e.g., of the multispecific antigen-binding protein.

In some embodiments, the masking moiety and cleavable linker are fused to the N-terminus of the heavy chain of the molecule, e.g., of the multispecific antigen-binding protein; such as, e.g., to the N-terminus of the first ISV specifically binding to 4-1BB.

In some embodiments, the molecule, e.g., the multispecific antigen-binding protein, comprises
(i) at least a first polypeptide with SEQ ID NO: 11 or 9, or an amino acid sequence sharing at least 70%, 75%, 80%, 85%, 90%, 95%, 96%, 97%, 98%, or 99% of sequence identity over the non-CDR regions of SEQ ID NO: 11 or 9; and
(ii) at least a second polypeptide with SEQ ID NO: 52, 53, 54 or 55, or an amino acid sequence sharing at least 70%, 75%, 80%, 85%, 90%, 95%, 96%, 97%, 98%, or 99% of sequence identity over the non-CDR regions of SEQ ID NO: 52, 53, 54 or 55; preferably an amino acid sequence sharing at least 70%, 75%, 80%, 85%, 90%, 95%, 96%, 97%, 98%, or 99% of sequence identity over the non-CDR, masking moiety and cleavable linker regions of SEQ ID NO: 52, 53, 54 or 55.

In some embodiments, the molecule, e.g., the multispecific antigen-binding protein, comprises at least a first polypeptide with SEQ ID NO: 11, and at least a second polypeptide with SEQ ID NO: 52.

In some embodiments, the molecule, e.g., the multispecific antigen-binding protein, comprises at least a first polypeptide with SEQ ID NO: 11, and at least a second polypeptide with SEQ ID NO: 53.

In some embodiments, the molecule, e.g., the multispecific antigen-binding protein, comprises at least a first polypeptide with SEQ ID NO: 11, and at least a second polypeptide with SEQ ID NO: 54.

In some embodiments, the molecule, e.g., the multispecific antigen-binding protein, comprises at least a first polypeptide with SEQ ID NO: 11, and at least a second polypeptide with SEQ ID NO: 55.

In some embodiments, the molecule, e.g., the multispecific antigen-binding protein, comprises at least a first polypeptide with SEQ ID NO: 9, and at least a second polypeptide with SEQ ID NO: 52.

In some embodiments, the molecule, e.g., the multispecific antigen-binding protein, comprises at least a first polypeptide with SEQ ID NO: 9, and at least a second polypeptide with SEQ ID NO: 53.

In some embodiments, the molecule, e.g., the multispecific antigen-binding protein, comprises at least a first polypeptide with SEQ ID NO: 9, and at least a second polypeptide with SEQ ID NO: 54.

In some embodiments, the molecule, e.g., the multispecific antigen-binding protein, comprises at least a first polypeptide with SEQ ID NO: 9, and at least a second polypeptide with SEQ ID NO: 55.

In some embodiments, the molecule, e.g., the multispecific antigen-binding protein, further comprises a third and fourth polypeptide identical to the first and second polypeptide, respectively.

### Antigen-Binding Fragments of Antibodies

Unless specifically indicated otherwise, the term “antibody”, as used herein, shall be understood to encompass antibody molecules comprising two immunoglobulin heavy chains and two immunoglobulin light chains (i.e., “full antibody molecules”) as well as antigen-binding fragments thereof. An antibody fragment may include a Fab fragment, a F(ab′)2 fragment, a Fv fragment, a dAb fragment, a fragment containing a CDR, or an isolated CDR. In certain embodiments, the term “antigen-binding fragment” refers to a polypeptide fragment of a multi-specific antigen-binding molecule. In such embodiments, the term “antigen-binding fragment” includes, e.g., an extracellular domain of PD-L1 which binds specifically to PD-1 or an extracellular domain of 4-1BBL which binds specifically to 4-1BB. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) VH-CH1; (ii) VH-CH2; (iii) VH-CH3; (iv) VH-CH1-CH2; (v) VH-CH1-CH2-CH3; (vi) VH-CH2-CH3; (vii) VH-CL; (viii) VL-CH1; (ix) VL-CH2; (X) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric VH or VL domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multi-specific antibody format, including the exemplary bi-specific antibody formats disclosed herein, may be adapted for use in the context of an antigen-binding fragment of an antibody of the present disclosure using routine techniques available in the art.

### Preparation of Human Antibodies

Methods for generating human antibodies in transgenic mice are known in the art. Any such known methods can be used in the context of the present disclosure to make human antibodies that specifically bind to PD-1 and/or 4-1BB.

An immunogen comprising any one of the following can be used to generate antibodies to PD-1 and/or 4-1BB. In certain embodiments, the antibodies of the disclosure are obtained from mice immunized with a full length, native PD-1 and/or 4-1BB, or with a recombinant PD-1 and/or 4-1BB peptide. Alternatively, PD-1 and/or 4-1BB or a fragment thereof may be produced using standard biochemical techniques and modified and used as immunogen. In certain embodiments, the immunogen may be a peptide from the N terminal or C terminal end of PD-1 and/or 4-1BB.

In some embodiments, the immunogen may be a recombinant PD-1 and/or 4-1BB peptide expressed in E. coli or in any other eukaryotic or mammalian cells such as Chinese hamster ovary (CHO) cells.

In certain embodiments, antibodies that bind specifically to PD-1 and/or 4-1BB may be prepared using fragments of the above-noted regions, or peptides that extend beyond the designated regions by about 5 to about 20 amino acid residues from either, or both, the N or C terminal ends of the regions described herein. In certain embodiments, any combination of the above-noted regions or fragments thereof may be used in the preparation of PD-1 and/or 4-1BB specific antibodies.

Using VelocImmune® technology (see, for example, U.S. Pat. No. 6,596,541, Regeneron Pharmaceuticals) or any other known method for generating monoclonal antibodies, high affinity chimeric antibodies to PD-1 and/or 4-1BB can be initially isolated having a human variable region and a mouse constant region. The VelocImmune® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody. As will be appreciated by a person skilled in the art, several other transgenic mouse systems may be used, such as the Trianni mouse from Trianni Inc., the KymouseR mouse from Kymab Limited, the OmniMouseR from OmniAb, or the HuMAb MouseR from Medarex, to cite a few.

### Bioequivalents

The anti-PD-1/anti-4-1BB bispecific antibodies of the present disclosure (or any of their individual constituents) encompass proteins having amino acid sequences that vary from those of the described antibodies, but that retain the ability to bind PD-1 and 4-1BB. Such variant antibodies and antigen-binding fragments thereof comprise one or more additions, deletions, or substitutions of amino acids when compared to parent sequence, but exhibit biological activity that is essentially equivalent to that of the described antibodies. Likewise, the antibody-encoding DNA sequences of the present disclosure encompass sequences that comprise one or more additions, deletions, or substitutions of nucleotides when compared to the disclosed sequence, but that encode an antibody or antibody fragment that is essentially bioequivalent to an antibody or antibody fragment of the disclosure.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single dose or multiple doses. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, or potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and/or in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the antibodies of the disclosure may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include antibody variants comprising amino acid changes, which modify the glycosylation characteristics of the antibodies, e.g., mutations that eliminate or remove glycosylation.

### Anti-PD-1/Anti-4-1BB Antibodies Comprising Fc Variants

According to certain embodiments of the present disclosure, anti-PD-1/anti-4-1BB antibodies are provided comprising an Fc domain comprising one or more mutations which enhance or diminish antibody binding to the FcRn receptor, e.g., at acidic pH as compared to neutral pH. For example, the present disclosure includes anti-PD-1/anti-4-1BB antibodies comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. In the following, and unless otherwise specified, numbering of amino acid residues in the Fc domain or constant region is made according to the so-called EU index, as described in Kabat et al., 1991 (Sequences of proteins of immunological interest. 5th Ed., Bethesda, MD: U.S. Dept. of Health and Human Services, Public Health Service, National Institutes of Health). Non-limiting examples of such Fc modifications include, e.g., a modification at position 234 (e.g., A), 235 (e.g., A), 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., A, W, H, F or Y [N434A, N434W, N434H, N434F or N434Y]); or a modification at position 250 and/or 428; or a modification at position 307 or 308 (e.g., 308F, V308F), and 434. In one embodiment, the modification comprises a 234A (e.g., L234A) and 235A (e.g., L235A) modification, 428L (e.g., M428L) and 434S (e.g., N434S) modification; a 428L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P). In yet another embodiment, the modification comprises a 265A (e.g., D265A) and/or a 297A (e.g., N297A) modification.

For example, the present disclosure includes anti-PD-1/anti-4-1BB antibodies comprising an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); 2571 and 3111 (e.g., P2571 and Q3111); 2571 and 434H (e.g., P2571 and N434H); 376V and 434H (e.g., D376V and N434H); 307A, 380A and 434A (e.g., T307A, E380A and N434A); 433K and 434F (e.g., H433K and N434F); and 234A and 235A (e.g., L234A and L235A). In one embodiment, the present disclosure includes anti-PD-1 antibodies comprising an Fc domain comprising a S108P mutation in the hinge region of IgG4 to promote dimer stabilization. All possible combinations of the foregoing Fc domain mutations, and other mutations within the antibody variable domains disclosed herein, are contemplated within the scope of the present disclosure.

The present disclosure also includes anti-PD-1/anti-4-1BB antibodies comprising a chimeric heavy chain constant (C_{H}) region, wherein the chimeric C_{H} region comprises segments derived from the C_{H} regions of more than one immunoglobulin isotype. For example, the antibodies of the disclosure may comprise a chimeric C_{H} region comprising part or all of a C_{H}2 domain derived from a human IgG1, human IgG2 or human IgG4 molecule, combined with part or all of a C_{H}3 domain derived from a human IgG1, human IgG2 or human IgG4 molecule. According to certain embodiments, the antibodies of the disclosure comprise a chimeric C_{H} region having a chimeric hinge region. For example, a chimeric hinge may comprise an “upper hinge” amino acid sequence (amino acid residues from positions 216 to 227 according to EU numbering) derived from a human IgG1, a human IgG2 or a human IgG4 hinge region, combined with a “lower hinge” sequence (amino acid residues from positions 228 to 236 according to EU numbering) derived from a human IgG1, a human IgG2 or a human IgG4 hinge region. According to certain embodiments, the chimeric hinge region comprises amino acid residues derived from a human IgG1 or a human IgG4 upper hinge and amino acid residues derived from a human IgG2 lower hinge. An antibody comprising a chimeric C_{H} region as described herein may, in certain embodiments, exhibit modified Fc effector functions without adversely affecting the therapeutic or pharmacokinetic properties of the antibody. (See, e.g., U.S. Pat. No. 9,359,437, the disclosure of which is hereby incorporated by reference in its entirety.)

### Biological Characteristics of the Antibodies

In general, the antibodies of the present disclosure function by binding to PD-1 and 4-1BB. The present disclosure includes anti-PD-1/anti-4-1BB bispecific antibodies and antigen-binding fragments thereof (or any of their individual constituents) that bind soluble monomeric or dimeric PD-1 and 4-1BB molecules with high affinity. For example, the present disclosure includes antibodies and antigen-binding fragments of antibodies that bind PD-1 and 4-1BB (e.g., at 25° C. or at 37° C.) with a K_{D} of less than about 50 nM as measured by surface plasmon resonance. In certain embodiments, the antibodies or antigen-binding fragments thereof bind PD-1 and 4-1BB with a K_{D} of less than about 40 nM, less than about 30 nM, less than about 20 nM, less than about 10 nM less than about 5 nM, less than about 2 nM or less than about 1 nM, as measured by surface plasmon resonance.

The present disclosure also includes antibodies and antigen-binding fragments thereof that bind PD-1 and 4-1BB with a dissociative half-life (t½) of greater than about 1.1 minutes as measured by surface plasmon resonance at 25° C. or 37° C. In certain embodiments, the antibodies or antigen-binding fragments of the present disclosure bind PD-1 with a t½ of greater than about 5 minutes, greater than about 10 minutes, greater than about 30 minutes, greater than about 50 minutes, greater than about 60 minutes, greater than about 70 minutes, greater than about 80 minutes, greater than about 90 minutes, greater than about 100 minutes, greater than about 200 minutes, greater than about 300 minutes, greater than about 400 minutes, greater than about 500 minutes, greater than about 600 minutes, greater than about 700 minutes, greater than about 800 minutes, greater than about 900 minutes, greater than about 1000 minutes, or greater than about 1200 minutes, as measured by surface plasmon resonance at 25° C. or 37° C.

### Species Selectivity and Species Cross-Reactivity

According to certain embodiments of the disclosure, the anti-PD-1/anti-4-1BB antibodies (or any of their individual constituents) bind to human PD-1 and human 4-1BB but not to PD-1 and 4-1BB from other species. Alternatively, the anti-PD-1/anti-4-1BB antibodies of the disclosure, in certain embodiments, bind to human PD-1 and human 4-1BB and to PD-1 and 4-1BB from one or more non-human species. For example, the anti-PD-1/anti-4-1BB antibodies of the disclosure may bind to human PD-1 and/or human 4-1BB and may bind or not bind, as the case may be, to one or more of mouse, rat, guinea pig, hamster, gerbil, pig, cat, dog, rabbit, goat, sheep, cow, horse, camel, cynomolgus, marmoset, rhesus or chimpanzee PD-1 and/or 4-1BB. In certain embodiments, the anti-PD-1/anti-4-1BB antibodies of the disclosure may bind to human and cynomolgus PD-1 and/or 4-1BB with the same affinities or with different affinities, but do not bind to rat and mouse PD-1 and/or 4-1BB.

### Therapeutic Administration and Formulations

The disclosure provides therapeutic compositions comprising the anti-PD-1/anti-4-1BB antibodies of the present disclosure (or any of their individual constituents). Therapeutic compositions in accordance with the disclosure will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures s containing carbowax. See also Powell et al., PDA J Pharm Sci Technol. 1998 September-October;52(5):238-311.

The dose of antibody may vary depending upon the age and the size of a subject to be administered, target disease, conditions, route of administration, and the like. When an antibody of the present disclosure is used for treating a disease or disorder in an adult patient, or for preventing such a disease, it is advantageous to administer the antibody of the present disclosure normally at a single dose of about 0.1 to about 60 mg/kg body weight, or about 5 to about 60 mg/kg body weight, about 10 to about 50 mg/kg body weight, or about 20 to about 50 mg/kg body weight. Depending on the severity of the condition, the frequency and the duration of the treatment can be adjusted. In certain embodiments, the antibody or antigen-binding fragment thereof of the disclosure can be administered as an initial dose of at least about 0.1 mg to about 800 mg, about 1 mg to about 500 mg, about 5 mg to about 300 mg, or about 10 mg to about 200 mg, to about 100 mg, or to about 50 mg. In certain embodiments, the initial dose may be followed by administration of a second or a plurality of subsequent doses of the antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., J Biol Chem. 1987 Apr. 5; 262(10):4429-32). Methods of introduction include, but are not limited to, intradermal, transdermal, intramuscular, intraperitoneal, intravenous, subcutaneous, intratumoral, intranasal, epidural and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. The pharmaceutical composition can be also delivered in a vesicle, in particular a liposome (see, for example, Langer. Science. 1990 Sep. 28; 249(4976): 1527-33).

The use of nanoparticles to deliver the antibodies of the present invention is also contemplated herein. Antibody-conjugated nanoparticles may be used both for therapeutic and diagnostic applications. Antibody-conjugated nanoparticles and methods of preparation and use are described in detail by Arruebo et al. (J Nanomat. 2009; pp. 1-24), incorporated herein by reference. Nanoparticles may be developed and conjugated to antibodies contained in pharmaceutical compositions to target tumor cells or virally infected cells. Nanoparticles for drug delivery have also been described in, for example, U.S. Pat. No. 8,257,740, or U.S. Pat. No. 8,246,995, each incorporated herein in its entirety.

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracutaneous, intracranial, intraperitoneal, intramuscular, and intratumoral injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is optionally filled in an appropriate ampule.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present disclosure. Examples include, but certainly are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (Sanofi-Aventis, Frankfurt, Germany), and the like. Examples of disposable pen delivery devices having applications in subcutaneous delivery of a pharmaceutical composition of the present disclosure include, but certainly are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L. P.) and the HUMIRA™ Pen (Abbott Labs, Abbott Park, Ill.), and the like.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the antibody contained is generally about 5 to about 500 mg per dosage form in a unit dose; especially in the form of injection, the antibody is contained in about 5 to about 100 mg and in about 10 to about 250 mg for the other dosage forms.

### Therapeutic Uses of the Antibodies

The antibodies of the disclosure (or any of their individual constituents) are useful, inter alia, for the treatment, prevention and/or amelioration of any disease or disorder associated with or mediated by PD-1 and/or 4-1BB expression, signaling, or activity, or treatable by blocking the interaction between PD-1 and a PD-1 ligand (e.g., PD-L1, or PD-L2), and 4-1BB and a 4-1BB ligand (e.g., 4-1BBL) or otherwise inhibiting PD-1 and activating 4-1BB activity and/or signaling. For example, the present disclosure provides methods for treating cancer (tumor growth inhibition) and/or chronic viral infections by administering an anti-PD-1/anti-4-1BB bispecific binding molecule (or pharmaceutical composition comprising an anti-PD-1/anti-4-1BB bispecific binding molecule) as described herein to a patient in need of such treatment. The antibodies of the present disclosure are useful for the treatment, prevention, and/or amelioration of disease or disorder or condition such as cancer or a viral infection and/or for ameliorating at least one symptom associated with such disease, disorder or condition. In the context of the methods of treatment described herein, the anti-PD-1/anti-4-1BB bispecific binding molecule may be administered as a monotherapy (i.e., as the only therapeutic agent) or in combination with one or more additional therapeutic agents (examples of which are described elsewhere herein).

In some embodiments of the disclosure, the antibodies described herein are useful for treating subjects suffering from primary or recurrent cancer, including, but not limited to, bladder cancer, bone cancer, brain cancer, breast cancer, cervical cancer, colon cancer, esophageal cancer, gastric cancer, head-and-neck cancer, leukemia, liver cancer, lung cancer, lymphoma, melanoma, mesothelioma, multiple myeloma, myelodysplastic syndrome, ovarian cancer, pancreatic cancer, prostate cancer, rectal cancer, renal/kidney cancer, sarcoma, skin cancer, testicular cancer, thyroid cancer, and uterine cancer.

The antibodies may be used to treat early stage or late-stage symptoms of cancer. In one embodiment, an antibody or fragment thereof of the disclosure may be used to treat metastatic cancer. The antibodies are useful in reducing or inhibiting or shrinking tumor growth of both solid tumors and blood cancers. In certain embodiments, treatment with an antibody or antigen-binding fragment thereof of the disclosure leads to more than 50% regression, more than 60% regression, more than 70% regression, more than 80% regression or more than 90% regression of a tumor in a subject. In certain embodiments, the antibodies may be used to prevent relapse of a tumor. In certain embodiments, the antibodies are useful in extending overall survival in a subject with cancer. In some embodiments, the antibodies are useful in reducing toxicity due to chemotherapy or radiotherapy while maintaining long-term survival in a patient suffering from cancer.

In certain embodiments, the antibodies of the disclosure are useful to treat subjects suffering from a chronic viral infection. In some embodiments, the antibodies of the invention are useful in decreasing viral titers in the host and/or rescuing exhausted T-cells. In certain embodiments, an antibody or fragment thereof of the disclosure may be used to treat chronic viral infection by lymphocytic choriomeningitis virus (LCMV). In some embodiments, an antibody or antigen-binding fragment thereof the disclosure may be administered at a therapeutic dose to a patient with an infection by human immunodeficiency virus (HIV) or human papilloma virus (HPV) or hepatitis B/C virus (HBV/HCV). In a related embodiment, an antibody or antigen-binding fragment thereof of the disclosure may be used to treat an infection by simian immunodeficiency virus (SIV) in a simian subject such as cynomolgus.

In certain embodiments, an antibody of the present disclosure may be administered in a therapeutically effective amount to a subject suffering from a cancer or a viral infection.

One or more antibodies of the present disclosure may be administered to relieve or prevent or decrease the severity of one or more of the symptoms or conditions of the disease or disorder.

It is also contemplated herein to use one or more antibodies of the present disclosure prophylactically to patients at risk for developing a disease or disorder such as cancer and chronic viral infection.

In a further embodiment of the disclosure the present antibodies are used for the preparation of a pharmaceutical composition for treating patients suffering from cancer or viral infection. In another embodiment of the disclosure, the present antibodies are used as adjunct therapy with any other agent or any other therapy known to those skilled in the art useful for treating cancer or viral infection.

### Combination Therapies and Formulations

Combination therapies may include an anti-PD-1/anti-4-1BB bispecific binding molecule of the disclosure (or any of their individual constituents) and any additional therapeutic agent that may be advantageously combined with an antibody of the disclosure.

The antibodies of the present disclosure may be combined synergistically with one or more anti-cancer drugs or therapy used to treat cancer, including, for example, bladder cancer, bone cancer, brain cancer, breast cancer, cervical cancer, colon cancer, esophageal cancer, gastric cancer, head-and-neck cancer, leukemia, liver cancer, lung cancer, lymphoma, melanoma, mesothelioma, multiple myeloma, myelodysplastic syndrome, ovarian cancer, pancreatic cancer, prostate cancer, rectal cancer, renal/kidney cancer, sarcoma, skin cancer, testicular cancer, thyroid cancer, and uterine cancer. It is contemplated herein to use anti-PD-1/anti-4-1BB antibodies of the disclosure in combination with immunostimulatory and/or immunosupportive therapies to inhibit tumor growth, and/or enhance survival of cancer patients. The immunostimulatory therapies include direct immunostimulatory therapies to augment immune cell activity by either “releasing the brake” on suppressed immune cells or “stepping on the gas” to activate an immune response. Examples include targeting other checkpoint receptors, adoptive cell therapy, vaccination and adjuvants. The immunosupportive modalities may increase antigenicity of the tumor by promoting immunogenic cell death, inflammation or have other indirect effects that promote an anti-tumor immune response. Examples include radiation, chemotherapy, anti-angiogenic agents, and surgery.

In various embodiments, one or more antibodies of the present disclosure may be used in combination with an antibody to PD-L1; a second antibody to PD-1 (e.g., nivolumab); an antibody to 4-1BBL; a second antibody to 4-1BB; a LAG-3 inhibitor; a CTLA-4 inhibitor (e.g., ipilimumab); a TIM-3 inhibitor; a BTLA inhibitor; a TIGIT inhibitor; a CD47 inhibitor; an antagonist of another T-cell co-inhibitor or ligand (e.g., an antibody to PD-L2, CEACAM, VISTA, LAIR-1, 2B4, B7-H3, B7-H4, KIR, A2aR, GAL9, or TGFR); an agonist of a T-cell co-stimulator (e.g., an antibody or a ligand to CD28, ICOS, OX40, CD27, B7, CD226, CRTAM, GITR, HVEM, BAFFR, BAFF, Light); adenosine; an indoleamine-2,3-dioxygenase (IDO) inhibitor; a vascular endothelial growth factor (VEGF) antagonist (e.g., a “VEGF-Trap” such as aflibercept or other VEGF-inhibiting fusion protein as set forth in U.S. Pat. No. 7,087,411, or an anti-VEGF antibody or antigen binding fragment thereof [e.g., bevacizumab, or ranibizumab] or a small molecule kinase inhibitor of VEGF receptor [e.g., sunitinib, sorafenib, or pazopanib]); an Ang2 inhibitor (e.g., nesvacumab); a transforming growth factor beta (TGFβ) inhibitor; an epidermal growth factor receptor (EGFR) inhibitor (e.g., erlotinib, cetuximab); an agonist to a co-stimulatory receptor (e.g., an agonist to glucocorticoid-induced TNFR-related protein); an antibody to a tumor-specific antigen (e.g., CA9, CA125, melanoma-associated antigen 3 [MAGE3], carcinoembryonic antigen [CEA], vimentin, tumor-M2-PK, prostate-specific antigen [PSA], mucin-1, MART-1, and CA19-9); a vaccine (e.g., Bacillus Calmette-Guerin, a cancer vaccine); an adjuvant to increase antigen presentation (e.g., granulocyte-macrophage colony-stimulating factor); a bispecific antibody (e.g., CD3×CD20 bispecific antibody, PSMA×CD3 bispecific antibody); a cytotoxin; a chemotherapeutic agent (e.g., dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, and vincristine); cyclophosphamide; radiotherapy; an IL-6R inhibitor (e.g., sarilumab); an IL-4R inhibitor (e.g., dupilumab); an IL-10 inhibitor; a cytokine such as IL-2, IL-7, IL-12, IL-21, and IL-15; an antibody-drug conjugate (ADC) (e.g., anti-CD19-DM4 ADC, and anti-DS6-DM4 ADC); an immunocytokine (e.g., an anti-FAP×IL-2v [e.g., RO6874281], anti-tenascin CxIL-2 [e.g., F16-IL2, a.k.a. teleukin], anti-GD2×IL-2 [e.g., hu14.18-IL2], anti-EDBxIL-2 [e.g., L19-IL2, a.k.a. darleukin], anti-EDB×TNF [e.g., L19-TNF, a.k.a. fibromun], anti-histone complexxIL-12 [e.g., NHS-IL12], anti-EDB×IL-12 [e.g., L19-IL12, a.k.a. dodekin], anti-CSPG4×IL-2, anti-EpCAMxIL-2, anti-CD20×IL2, anti-PD-1×IL-2, and anti-TNFαxIL-2); an anti-inflammatory drug (e.g., corticosteroids, and non-steroidal anti-inflammatory drugs); a dietary supplement such as anti-oxidants; or any palliative care to treat cancer. In certain embodiments, the anti-PD-1 antibodies of the present disclosure may be used in combination with cancer vaccines (including dendritic cell vaccines, oncolytic viruses, tumor cell vaccines, etc.), or adoptive cell therapies, to augment the anti-tumor response. Examples of cancer vaccines that can be used in combination with anti-PD-1 antibodies of the present disclosure include MAGE3 vaccine for melanoma and bladder cancer, MUCI vaccine for breast cancer, EGFRv3 (e.g., Rindopepimut) for brain cancer (including glioblastoma multiforme), or ALVAC-CEA (for CEA cancers).

In certain embodiments, the anti-PD-1/anti-4-1BB antibodies of the disclosure may be administered in combination with radiation therapy in methods to generate long-term durable anti-tumor responses and/or enhance survival of patients with cancer. In some embodiments, the anti-PD-1/anti-4-1BB antibodies of the disclosure may be administered prior to, concomitantly or after administering radiation therapy to a cancer patient. For example, radiation therapy may be administered in one or more doses to tumor lesions followed by administration of one or more doses of anti-PD-1/anti-4-1BB antibodies of the disclosure. In some embodiments, radiation therapy may be administered locally to a tumor lesion to enhance the local immunogenicity of a patient's tumor (adjuvinating radiation) and/or to kill tumor cells (ablative radiation) followed by systemic administration of an anti-PD-1/anti-4-1BB bispecific binding molecule of the disclosure. For example, intracranial radiation may be administered to a patient with brain cancer (e.g., glioblastoma multiforme) in combination with systemic administration of an anti-PD-1/anti-4-1BB bispecific binding molecule of the disclosure. In certain embodiments, the anti-PD-1/anti-4-1BB antibodies of the disclosure may be administered in combination with radiation therapy and a chemotherapeutic agent (e.g., temozolomide) or a VEGF antagonist (e.g., aflibercept).

In certain embodiments, the anti-PD-1/anti-4-1BB antibodies of the disclosure may be administered in combination with one or more anti-viral drugs to treat chronic viral infection caused by LCMV, HIV, HPV, HBV or HCV. Examples of anti-viral drugs include, but are not limited to, zidovudine, lamivudine, abacavir, ribavirin, lopinavir, efavirenz, cobicistat, tenofovir, rilpivirine and corticosteroids. In some embodiments, the anti-PD-1/anti-4-1BB antibodies of the disclosure may be administered in combination with a LAG3 inhibitor, a CTLA-4 inhibitor or any antagonist of another T-cell co-inhibitor to treat chronic viral infection.

The additional therapeutically active agent(s)/component(s) may be administered prior to, concurrent with, or after the administration of the anti-PD-1/anti-4-1BB bispecific binding molecule of the disclosure. For purposes of the present disclosure, such administration regimens are considered the administration of an anti-PD-1/anti-4-1BB bispecific binding molecule “in combination with” a second therapeutically active component.

The additional therapeutically active component(s) may be administered to a subject prior to administration of an anti-PD-1/anti-4-1BB bispecific binding molecule of the disclosure. For example, a first component may be deemed to be administered “prior to” a second component if the first component is administered 1 week before, 72 hours before, 60 hours before, 48 hours before, 36 hours before, 24 hours before, 12 hours before, 6 hours before, 5 hours before, 4 hours before, 3 hours before, 2 hours before, 1 hour before, 30 minutes before, 15 minutes before, 10 minutes before, 5 minutes before, or less than 1 minute before administration of the second component. In other embodiments, the additional therapeutically active component(s) may be administered to a subject after administration of anti-PD-1/anti-4-1BB bispecific binding molecule of the disclosure. For example, a first component may be deemed to be administered “after” a second component if the first component is administered 1 minute after, 5 minutes after, 10 minutes after, 15 minutes after, 30 minutes after, 1 hour after, 2 hours after, 3 hours after, 4 hours after, 5 hours after, 6 hours after, 12 hours after, 24 hours after, 36 hours after, 48 hours after, 60 hours after, 72 hours after administration of the second component. In yet other embodiments, the additional therapeutically active component(s) may be administered to a subject concurrent with administration of an anti-PD-1/anti-4-1BB bispecific binding molecule of the disclosure. “Concurrent” administration, for purposes of the present disclosure, includes, e.g., administration of an anti-PD-1/anti-4-1BB bispecific binding molecule and an additional therapeutically active component to a subject in a single dosage form (e.g., co-formulated), or in separate dosage forms administered to the subject within about 30 minutes or less of each other. If administered in separate dosage forms, each dosage form may be administered via the same route (e.g., both the anti-PD-1/anti-4-1BB bispecific binding molecule and the additional therapeutically active component may be administered intravenously, subcutaneously, intratumorally, etc.); alternatively, each dosage form may be administered via a different route (e.g., the anti-PD-1/anti-4-1BB bispecific binding molecule may be administered intravenously, and the additional therapeutically active component may be administered subcutaneously or intratumorally; or the anti-PD-1/anti-4-1BB bispecific binding molecule may be administered intratumorally, and the additional therapeutically active component may be administered intravenously or subcutaneously; etc.). In any event, administering the components in a single dosage from, in separate dosage forms by the same route, or in separate dosage forms by different routes are all considered “concurrent administration,” for purposes of the present disclosure. For purposes of the present disclosure, administration of an anti-PD-1/anti-4-1BB bispecific binding molecule “prior to”, “concurrent with,” or “after” (as those terms are defined herein above) administration of an additional therapeutically active component is considered administration of an anti-PD-1/anti-4-1BB bispecific binding molecule “in combination with” an additional therapeutically active component).

The present disclosure includes pharmaceutical compositions in which an anti-PD-1/anti-4-1BB bispecific binding molecule of the disclosure is co-formulated with one or more of the additional therapeutically active component(s) as described elsewhere herein using a variety of dosage combinations.

### Administrative Regimens

According to certain embodiments of the present disclosure, multiple doses of an anti-PD-1/anti-4-1BB antibody of the disclosure (or any of their individual constituents)—or a pharmaceutical composition comprising a combination of an anti-PD-1 antibody and any of the additional therapeutically active agents mentioned herein—may be administered to a subject over a defined time course. The methods according to this aspect of the disclosure comprise sequentially administering to a subject multiple doses of an anti-PD-1/anti-4-1BB antibody of the disclosure. As used herein, “sequentially administering” means that each dose of anti-PD-1/anti-4-1BB antibody is administered to the subject at a different point in time, e.g., on different days separated by a predetermined interval (e.g., hours, days, weeks or months). The present disclosure includes methods which comprise sequentially administering to the patient a single initial dose of an anti-PD-1/anti-4-1BB antibody, followed by one or more secondary doses of the anti-PD-1/anti-4-1BB antibody, and optionally followed by one or more tertiary doses of the anti-PD-1 antibody. The anti-PD-1/anti-4-1BB antibody may be administered at a dose between 0.1 mg/kg to 100 mg/kg.

The terms “initial dose,” “secondary doses,” and “tertiary doses,” refer to the temporal sequence of administration of the anti-PD-1/anti-4-1BB antibody of the disclosure. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of anti-PD-1/anti-4-1BB antibody, but generally may differ from one another in terms of frequency of administration. In certain embodiments, however, the amount of anti-PD-1/anti-4-1BB antibody contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In certain exemplary embodiments of the present disclosure, each secondary and/or tertiary dose is administered 1 to 26 (e.g., 1, 11/2, 2, 21/2, 3, 31/2, 4, 41/2, 5, 51/2, 6, 61/2, 7, 71/2, 8, 81/2, 9, 91/2, 10, 101/2, 11, 111/2, 12, 121/2, 13, 131/2, 14, 141/2, 15, 151/2, 16, 161/2, 17, 171/2, 18, 181/2, 19, 191/2, 20, 201/2, 21, 211/2, 22, 221/2, 23, 231/2, 24, 241/2, 25, 251/2, 26, 261/2, or more) weeks after the immediately preceding dose. The phrase “the immediately preceding dose,” as used herein, means, in a sequence of multiple administrations, the dose of anti-PD-1/anti-4-1BB antibody which is administered to a patient prior to the administration of the very next dose in the sequence with no intervening doses.

The methods according to this aspect of the invention may comprise administering to a patient any number of secondary and/or tertiary doses of an anti-PD-1/anti-4-1BB antibody. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks or 1 to 2 months after the immediately preceding dose. Similarly, in embodiments involving multiple tertiary doses, each tertiary dose may be administered at the same frequency as the other tertiary doses. For example, each tertiary dose may be administered to the patient 2 to 12 weeks after the immediately preceding dose. In certain embodiments of the invention, the frequency at which the secondary and/or tertiary doses are administered to a patient can vary over the course of the treatment regimen. The frequency of administration may also be adjusted during the course of treatment by a physician depending on the needs of the individual patient following clinical examination.

The present disclosure includes administration regimens in which 2 to 6 loading doses are administered to a patient at a first frequency (e.g., once a week, once every two weeks, once every three weeks, once a month, once every two months, etc.), followed by administration of two or more maintenance doses to the patient on a less frequent basis. For example, according to this aspect of the disclosure, if the loading doses are administered at a frequency of, e.g., once a month (e.g., two, three, four, or more loading doses administered once a month), then the maintenance doses may be administered to the patient once every five weeks, once every six weeks, once every seven weeks, once every eight weeks, once every ten weeks, once every twelve weeks, etc.).

### Diagnostic Uses of the Antibodies

The anti-PD-1/anti-4-1BB bispecific binding molecules of the disclosure (or any of their individual constituents, i.e., an anti-4-1BB immunoglobulin single variable domain, or an anti-PD-1 antibody or antigen-binding fragment thereof) may be used to detect and/or measure PD-1 and/or 4-1BB in a sample, e.g., for diagnostic purposes. Some embodiments contemplate the use of one or more binding molecules of the present invention in assays to detect a disease or disorder such as cancer or chronic viral infection. Exemplary diagnostic assays for PD-1 and/or 4-1BB may comprise, e.g., contacting a sample, obtained from a patient, with an anti-PD-1/anti-4-1BB bispecific binding molecule of the disclosure (or one of its individual constituent), wherein the anti-PD-1/anti-4-1BB bispecific binding molecule (or one of its individual constituents) is labeled with a detectable label or reporter molecule or used as a capture ligand to selectively isolate PD-1 and/or 4-1BB from patient samples. Alternatively, an unlabeled anti-PD-1/anti-4-1BB bispecific binding molecule of the disclosure (or one of its individual constituent) can be used in diagnostic applications in combination with a secondary antibody which is itself detectably labeled. The detectable label or reporter molecule can be a radioisotope, such as ³H, ¹⁴C, ³²P, ³⁵S, or ¹²⁵I; a fluorescent or chemiluminescent moiety such as fluorescein isothiocyanate, or rhodamine; or an enzyme such as alkaline phosphatase, β-galactosidase, horseradish peroxidase, or luciferase. Specific exemplary assays that can be used to detect or measure PD-1 and/or 4-1BB in a sample include enzyme-linked immunosorbent assay (ELISA), radioimmunoassay (RIA), and fluorescence-activated cell sorting (FACS).

Samples that can be used in PD-1 and/or 4-1BB diagnostic assays according to the present disclosure include any tissue or fluid sample obtainable from a patient, which contains detectable quantities of PD-1 protein and/or of 4-1BB protein, or fragments thereof, under normal or pathological conditions. Generally, levels of PD-1 and/or of 4-1BB in a particular sample obtained from a healthy patient (e.g., a patient not afflicted with cancer) will be measured to initially establish a baseline, or standard, level of PD-1 and/or of 4-1BB. This baseline level of PD-1 and/or of 4-1BB can then be compared against the levels of PD-1 and/or of 4-1BB measured in samples obtained from individuals suspected of having a cancer-related condition, or symptoms associated with such condition.

The anti-PD-1/anti-4-1BB bispecific binding molecules of the disclosure (or any of their individual constituents, i.e., an anti-4-1BB immunoglobulin single variable domain, or an anti-PD-1 antibody or antigen-binding fragment thereof) may contain no additional labels or moieties, or they may contain an N-terminal or C-terminal label or moiety. In one embodiment, the label or moiety is biotin. In a binding assay, the location of a label (if any) may determine the orientation of the peptide relative to the surface upon which the peptide is bound. For example, if a surface is coated with avidin, a peptide containing an N-terminal biotin will be oriented such that the C-terminal portion of the peptide will be distal to the surface.

The present disclosure is further illustrated by the following examples which should not be construed as further limiting. The contents of the figures and all references, patents and published patent applications cited throughout this application are expressly incorporated herein by reference for all purposes.

Furthermore, in accordance with the present disclosure there may be employed conventional molecular biology, microbiology, and recombinant DNA techniques within the skill of the art. Such techniques are explained fully in the literature. See, e.g., Green & Sambrook, Molecular Cloning: A Laboratory Manual, Fourth Edition (2012) Cold Spring Harbor Laboratory Press, Cold Spring Harbor, New York; DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

### EXAMPLES

The following examples are put forth so as to provide those of ordinary skill in the art with a complete disclosure and description of how to make and use the methods and compositions featured in the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Anti-4-1BB V_{HH}s

### Material and Methods

### Llama Immunization and Library Construction

Immunization and library construction were conducted at the “VIB Nanobody Core” (Vrije Universiteit Brussel, Brussels, Belgium). A llama was injected intradermally four times, each time with approximately 2 mg of vector harboring the 4-1BB gene. After each injection, the animal was electroporated to introduce the vectors into animal cells. Three weeks after the last vector injection, the animal was subcutaneously boosted with recombinant 4-1BB protein. Four days after the protein boost, anticoagulated blood was collected for V_{HH} library construction.

The V_{HH} library was constructed. Total ribonucleic acid was extracted from peripheral blood lymphocytes and used as a template for first strand complementary DNA synthesis with oligo(dT) primers. V_{HH}-encoding sequences were amplified by polymerase chain reaction from complementary DNA, digested with PstI and NotI, and cloned between the PstI and NotI sites of a phagemid vector pHEN4 upstream of a decapeptide human influenza hemagglutinin-tag. A V_{HH} library of approximately 10⁸ independent transformants was obtained.

### Phage Display Panning

A bacteria library was grown in a 2YTAG medium (2×YT medium, 100 μg/mL ampicillin, 2% glucose) until absorbance at 600 nm (OD₆₀₀) reached 0.5, then infected with M13K07 helper phage (Invitrogen). After centrifugation, bacteria were resuspended in 2YTAK medium (2×YT medium, 100 μg/mL ampicillin, 50 μg/mL kanamycin) and grown overnight. Phage particles were precipitated from the culture supernatant by addition of 20% w/v polyethylene glycol 8000 (PEG8000) and 2.5 M NaCl, centrifuged, and resuspended in phosphate-buffered saline (PBS). Phages were subjected to one more wash and precipitation step, and were finally resuspended in cold PBS/15% v/v glycerol.

### Panning on Cells

Panning was performed at 4° ° C. on 4-1BB-transfected FreeStyle™ HEK293-FS cells. The phage-V_{HH} library was saturated in PBS/2% w/v BSA and incubated with 2×10⁷ cells for 2 hours at 4° C. After two washes with PBS, the cell pellet was resuspended in PBS and loaded on a fetal bovine serum/Percoll gradient. After centrifugation, the cell layer was collected and washed twice with PBS. Recovered cells with bound phages were added into a second fetal bovine serum/Percoll gradient and washed before mechanical lysis using beads (Dynabeads, Invitrogen). Recovered phage-V_{HH}s were used to infect exponentially growing Escherichia coli TG1 bacteria and either amplified overnight in 2YTAG medium for a new round of panning or plated on 2YTAG plates.

### Fab-Like Construction, Production, and Purification

After amplification with polymerase chain reaction, complementary DNA of the anti-4-1BB V_{HH}s were cloned into a proprietary mammalian expression vector in frame with either a human C_{L} domain or a human IgG1 C_{H}1 domain, fused to human influenza hemagglutinin and 6-His tags (both tags for purification purposes). The same was performed with anti-OX40 and anti-CD28 V_{HH}s, as well as with anti-foot-and-mouth disease virus (FMDV) V_{HH}, the later used as control as detailed later.

Plasmids were purified using NucleoBond Macherey-Nagel kits and Sanger sequenced. Bivalent monoparatopic (i.e., twice the same anti-4-1BB V_{HH}), bivalent biparatopic (i.e., two different anti-4-1BB V_{HH}s) and bispecific (i.e., one anti-4-1BB V_{HH} and one V_{HH} directed to another target) Fab-like constructs were produced by co-transfecting FreeStyle™ HEK293-FS cells with a mix of two plasmids encoding two V_{HH}s, with one fused to the human C_{L} domain and the other to the human IgG1 C_{H}1 domain. Supernatants were harvested 7 days later, purified on nickel affinity columns, and analyzed on CALIPER GXII (Perkin Elmer).

### Phage-V_{HH} Production in 96-Well Plates for ELISA

Individual TG1 colonies of 4-1BB V_{HH}s of interest were grown in 2YTA medium at 37° C. until OD₆₀₀ reached 0.5. Cells were then infected with the M13K07 helper phage and grown overnight in 2YTAK at 30° C. Supernatants containing phage-V_{HH}s were harvested and used for testing.

### ELISA Binding Assays

ELISA assays were performed on Nunc® MaxiSorp™ 96-well plates (Sigma) pre-coated overnight with 1 μg/mL of human His-tagged 4-1BB recombinant protein in PBS at 4° C. and further saturated with PBS/2% milk for 1 hour at room temperature. Bacteria supernatants containing phage-V_{HH}s or purified Fab-like constructs or 4-1BBL-Fc were incubated for 1 hour at room temperature. After several washes in PBS/0.1% Tween, the following HRP-conjugated antibodies were added: anti-HA-tag mAb (Sigma) for detection of bound Fab-like constructs, anti-M13 mAb (Santa Cruz Biotechnology) to detect bound phage-V_{HH}s and anti-human Fc mAb to detect bound 4-1BBL-His. Detection of peroxidase activity was performed using TMB (3,3′,5,5′-tetramethylbenzidine, KPL) substrate and OD_{450 nm} was measured on a SpectraMax microplate reader after addition of sulfuric acid stop solution.

### ELISA Competition Assays

ELISA assays were performed on Nunc MaxiSorp™ 96-well plates (Sigma) pre-coated overnight with 1 μg/mL of human His-tagged 4-1BB recombinant protein in PBS at 4° C. and further saturated with PBS/2% milk for 1 hour at room temperature. For epitope binning competition, serial dilutions of bivalent Fab-like constructs were incubated for 1 hour at room temperature then phage-V_{HH}s at their EC₉₀ were added and incubation was extended by 45 minutes at room temperature. Alternatively for competition assays, serial dilutions of a control anti-4-1BB antibody or 4-1BBL-Fc were incubated for 1 hour at room temperature before adding bivalent Fab-like constructs at a concentration corresponding to their EC₉₀ for 45 minutes at room temperature. After several washes in PBS/0.1% Tween, the following HRP-conjugated antibodies were added: anti-HA tag mAb (Sigma) for detection of bound Fab-like constructs and anti-M13 mAb (Santa Cruz Biotechnology) to detect bound phage-V_{HH}s. Detection of peroxidase activity was performed using TMB substrate and OD_{450 nm} was measured on a SpectraMax microplate reader after addition of sulfuric acid stop solution.

### Reporter Functional Assay

### For Cross-Linking Experiments

Test compounds were pre-incubated with a saturating concentration of anti-human Fab (Sigma, I5260) for 30 minutes at room temperature. Jurkat-4-1BB-NF-κB (Promega) cells were harvested during their exponential growth phase, and 25 μL of the cell suspension was added into a 96-well plate (50 000 cells/well) with 25 μL of cross-linked or non-cross-linked test compounds.

For Conditions with OX40-Expressing Cells

Jurkat-4-1BB-NF-κB cells were harvested during their exponential growth phase and mixed with OX40-expressing cells to obtain a final ratio of 1:1 between reporter and accessory cells. 25 μL of the cell suspension was added into a 96-well plate (50 000 cells/well) with 25 μL of test compounds.

### For all Conditions

The plate was incubated for 6 hours in a humidified incubator at 37° ° C. with 5% CO₂. 50 ML of Bio-Glo™ (Promega, G7941) reagent prepared according to the manufacturer's instructions was then added to each well and mixed. At least 5 minutes were allowed for complete cell lysis to occur, following which luminescence was measured using the Envision multimode plate reader (Perkin Elmer).

### Results

### Generation of Anti-4-1BB V_{HH}s

Anti-4-1BB V_{HH}s were obtained from llamas. More specifically, V_{HH}s were selected from llama immune and naive V_{HH} libraries. One llama was immunized with 4-1BB-DNA before building the V_{HH} phage library from PBMCs (i.e., the immune library). Llama immunization was performed by 6 injections of plasmids expressing full-length human 4-1BB; a final boost was administered by one injection of recombinant human 4-1BB (SinoBiological).

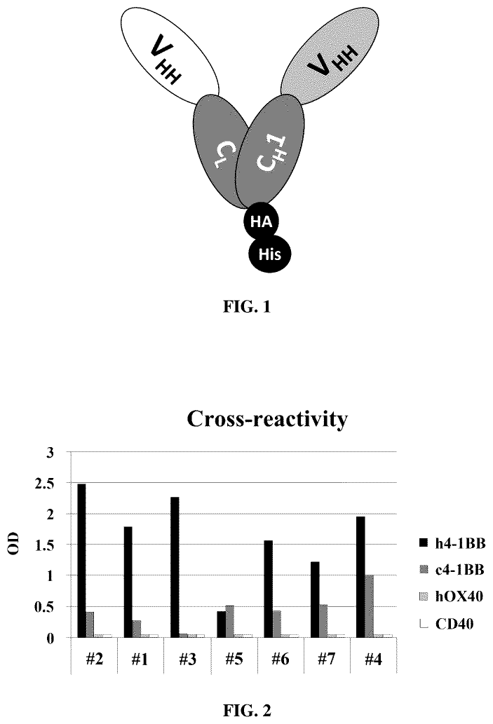

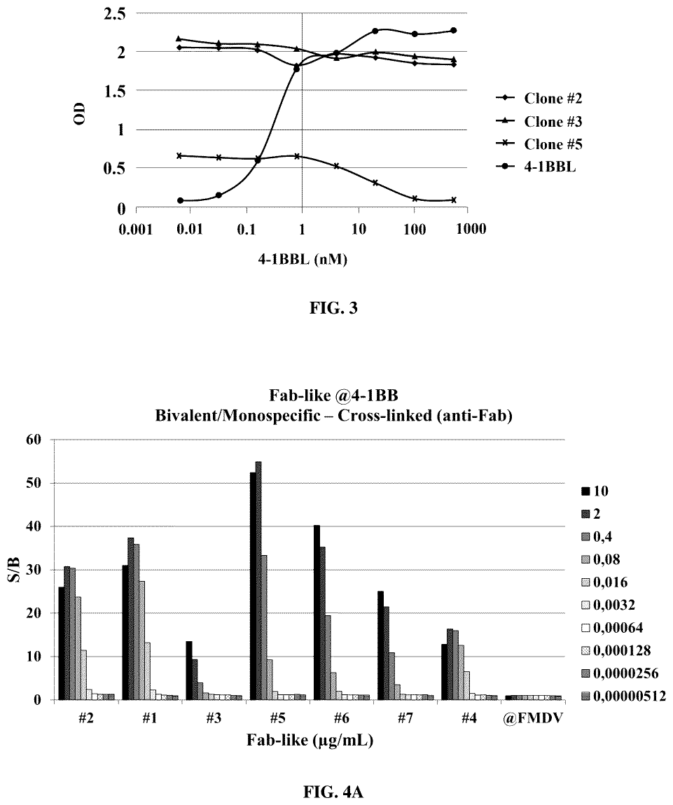

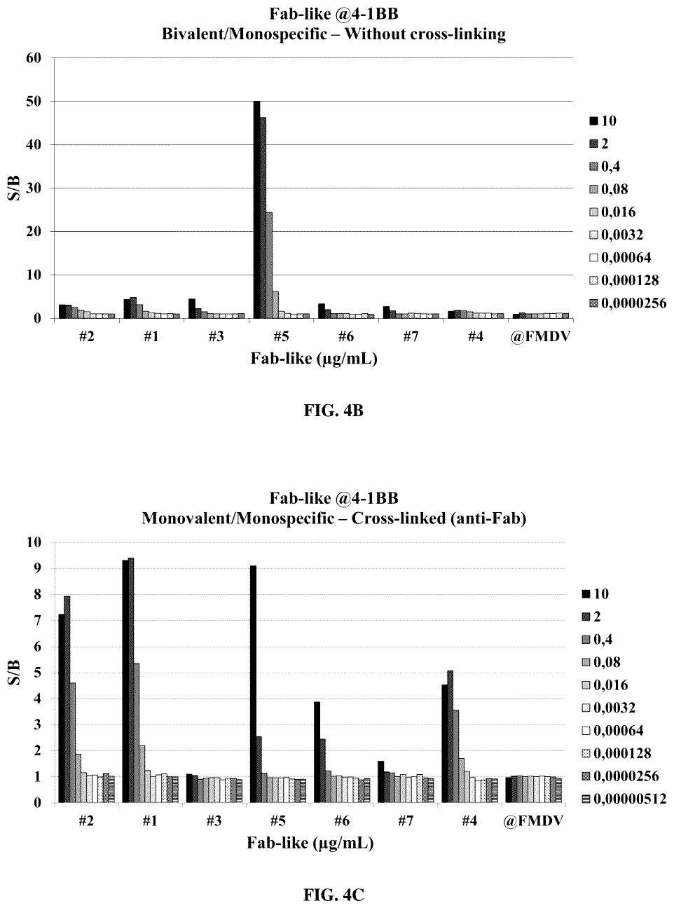

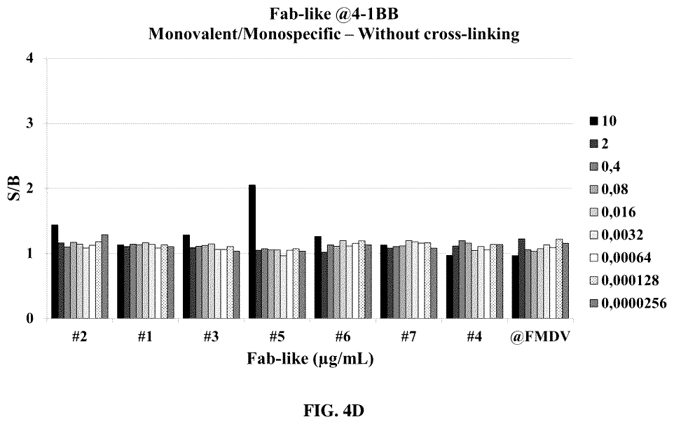

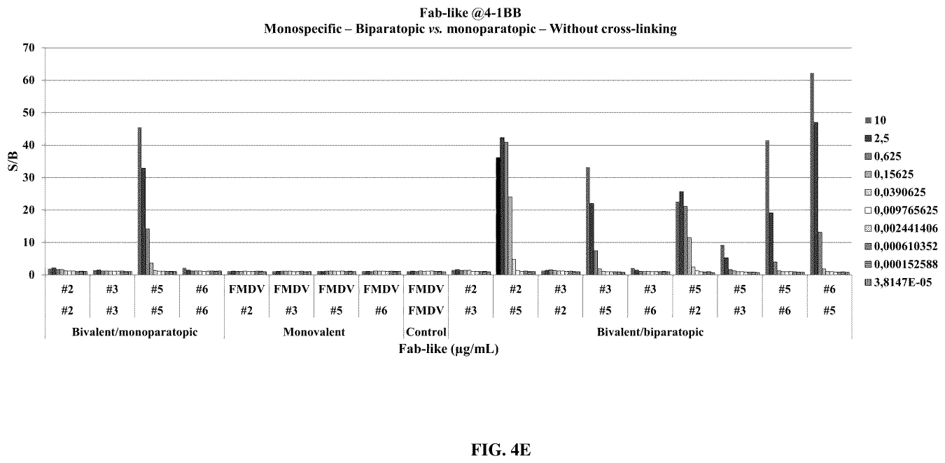

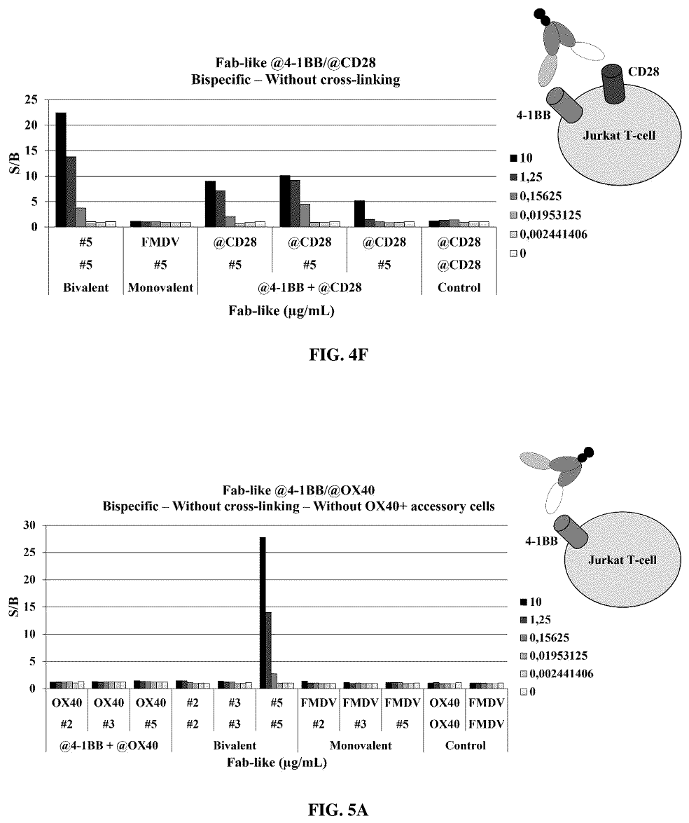

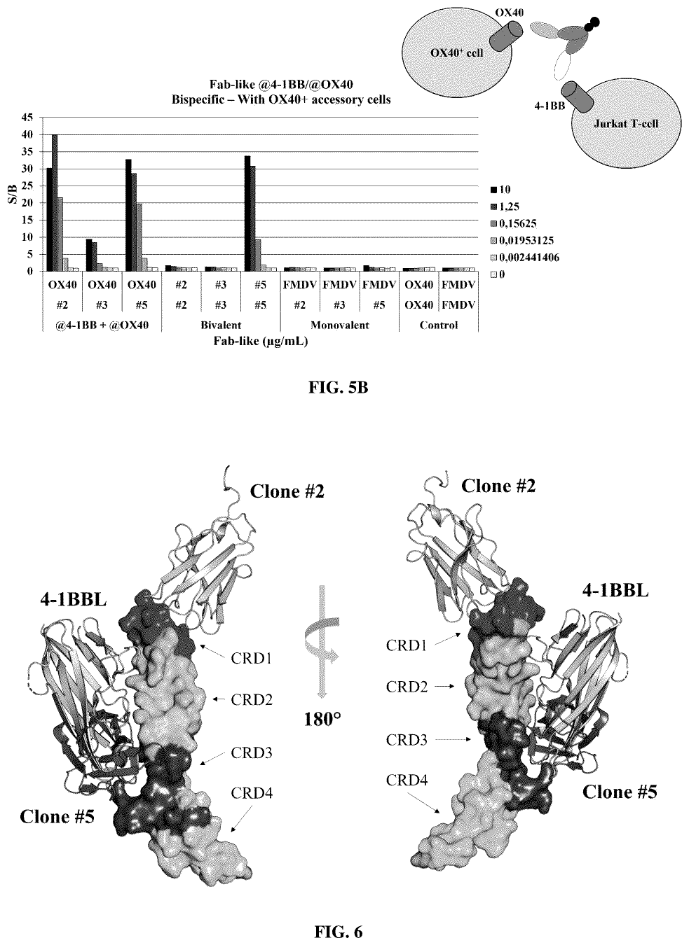

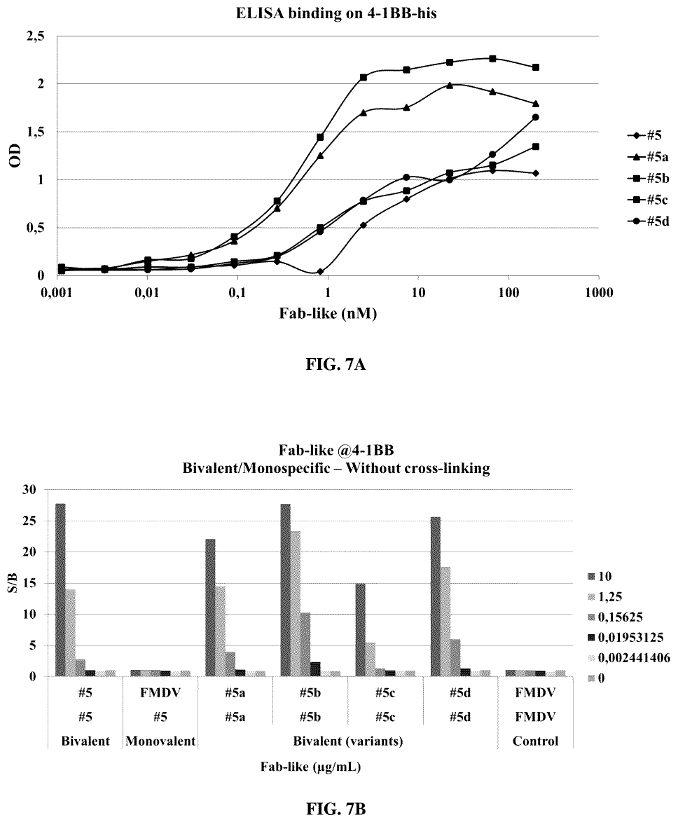

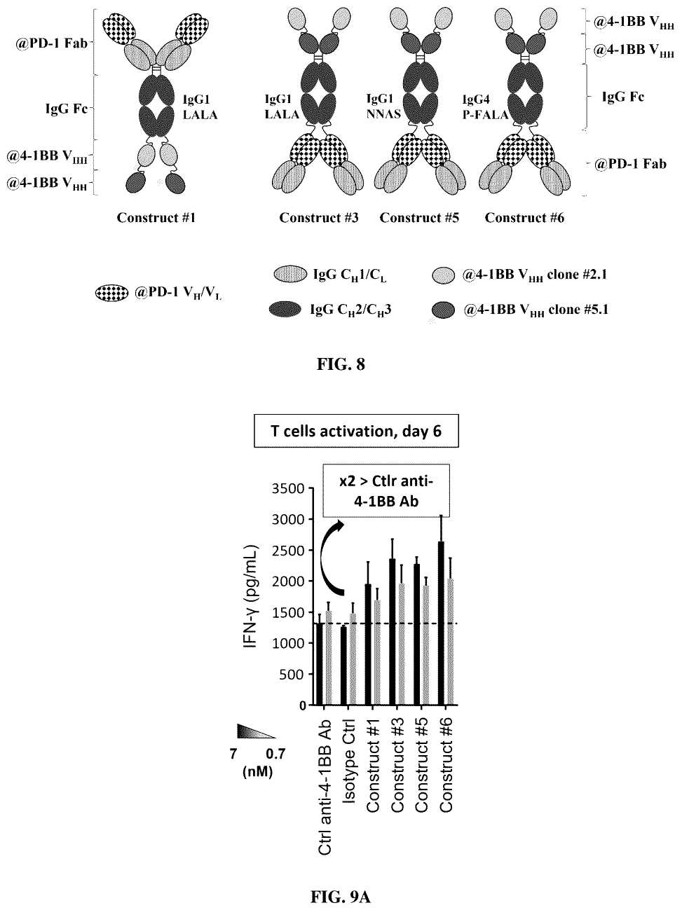

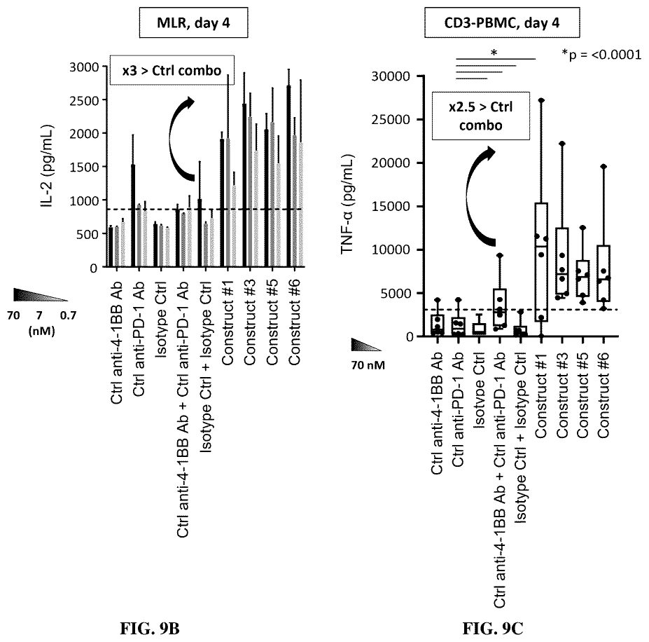

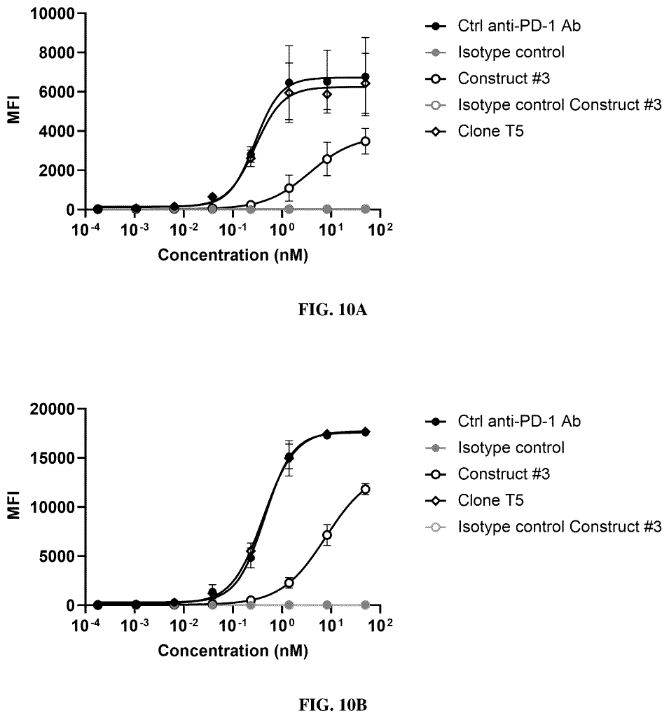

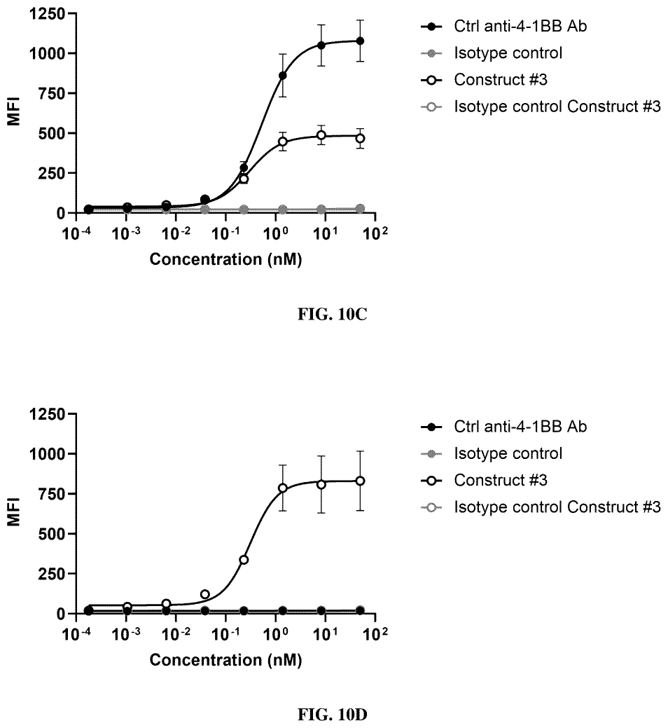

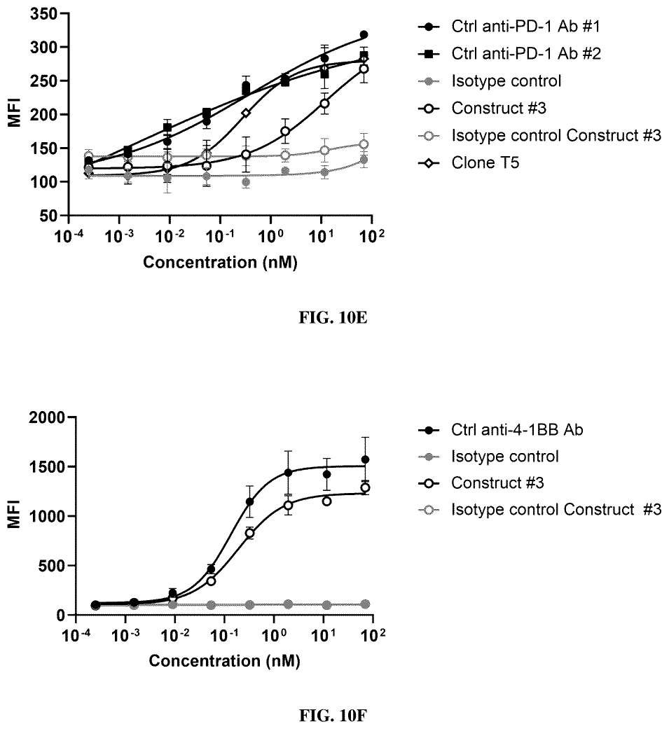

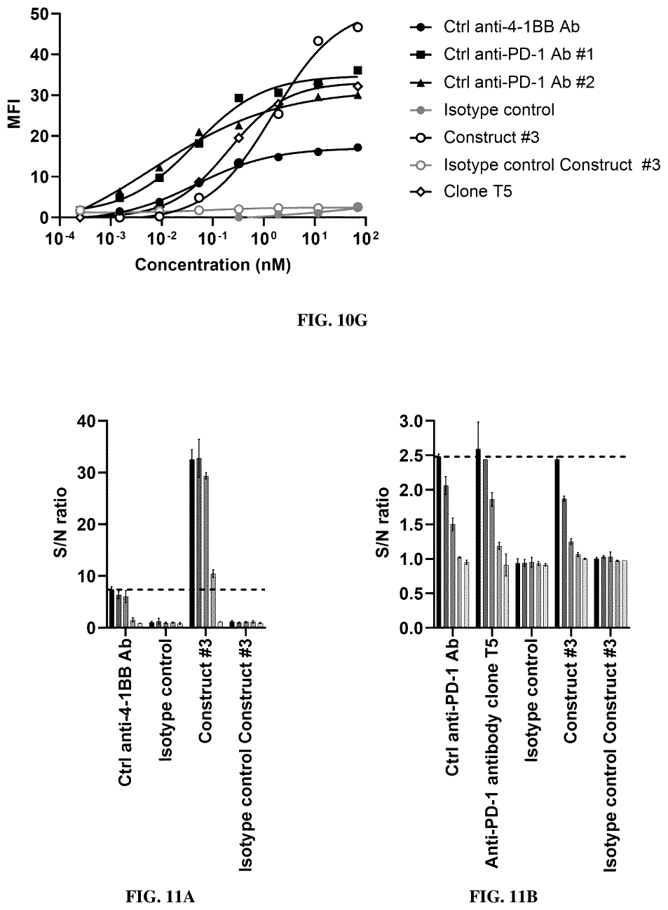

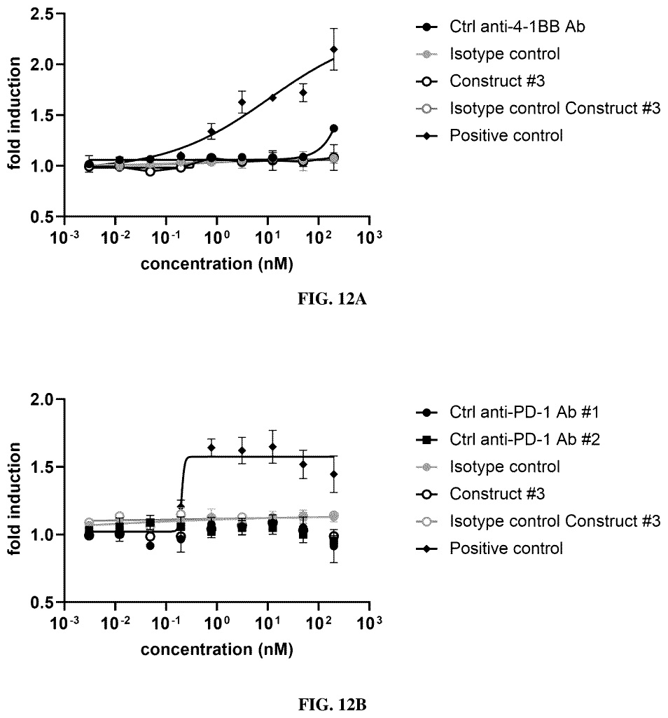

V_{HH}s selected by library panning on recombinant 4-1BB protein or 4-1BB-expressing cells were screened for binding to 4-1BB and further sequenced. Among binders, 7 clones (clones #1 to #7) were further characterized. These seven anti-4-1BB V_{HH}s were reformatted into bivalent monoparatopic (i.e., twice the same anti-4-1BB V_{HH}), bivalent biparatopic (i.e., two different anti-4-1BB V_{HH}s), bispecific (i.e., one anti-4-1BB V_{HH} and one V_{HH} directed to another target), or monovalent (i.e., one anti-4-1BB V_{HH} and one V_{HH} directed to an irrelevant target, e.g., FMDV) molecules, into a “Fab-like” format wherein a first V_{HH} is fused to a C_{H}1 human IgG constant domain and a second V_{HH} is fused to a C_{L} lambda human IgG constant domain; both first and second V_{HH} associate through C_{H}1-C_{L} interaction (FIG. 1). For purification purposes, the molecules comprise a hemagglutinin (HA)-tag and a polyhistidine (His)-tag.

### Specificity of Anti-4-1BB V_{HH}s

The specificity of the seven V_{HH}s (in bivalent monoparatopic “Fab-like” constructs each comprising twice a same anti-4-1BB V_{HH}) was evaluated by testing their binding to human 4-1BB, cynomolgus monkey (“cyno”) 4-1BB, and two TNFRSF members (human OX40 and human CD40).

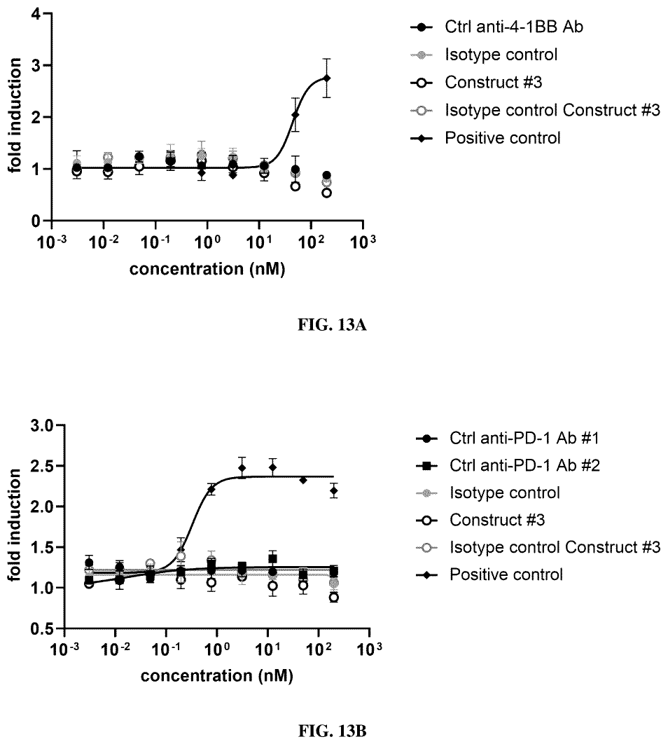

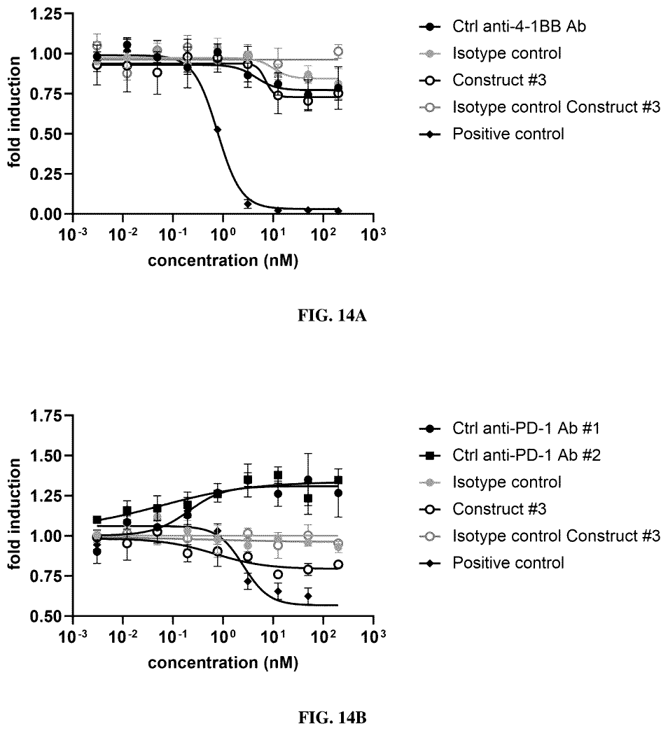

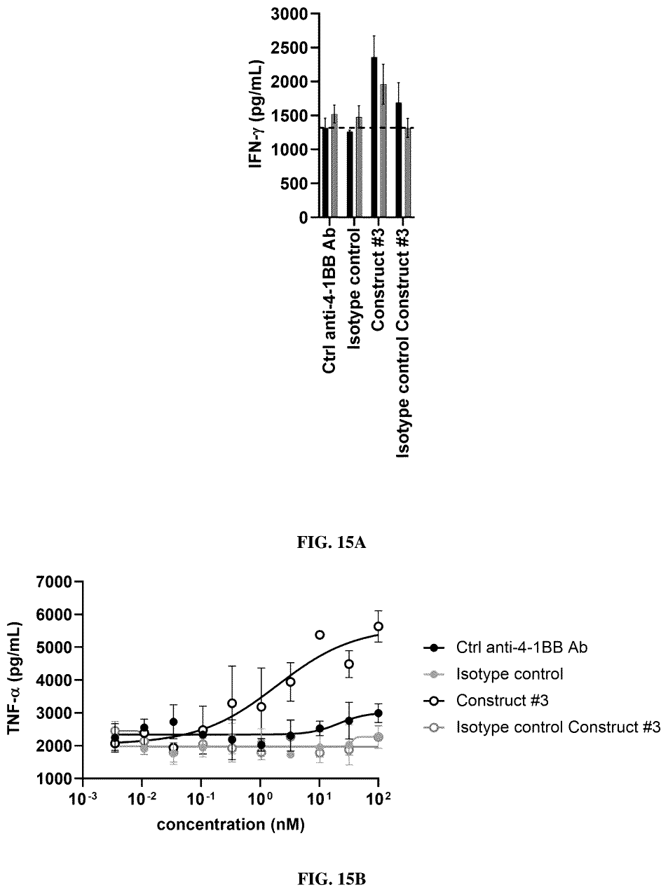

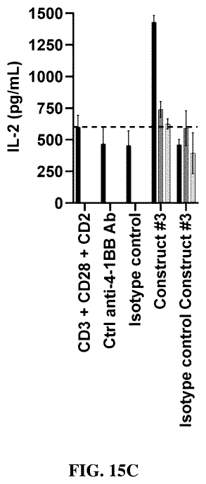

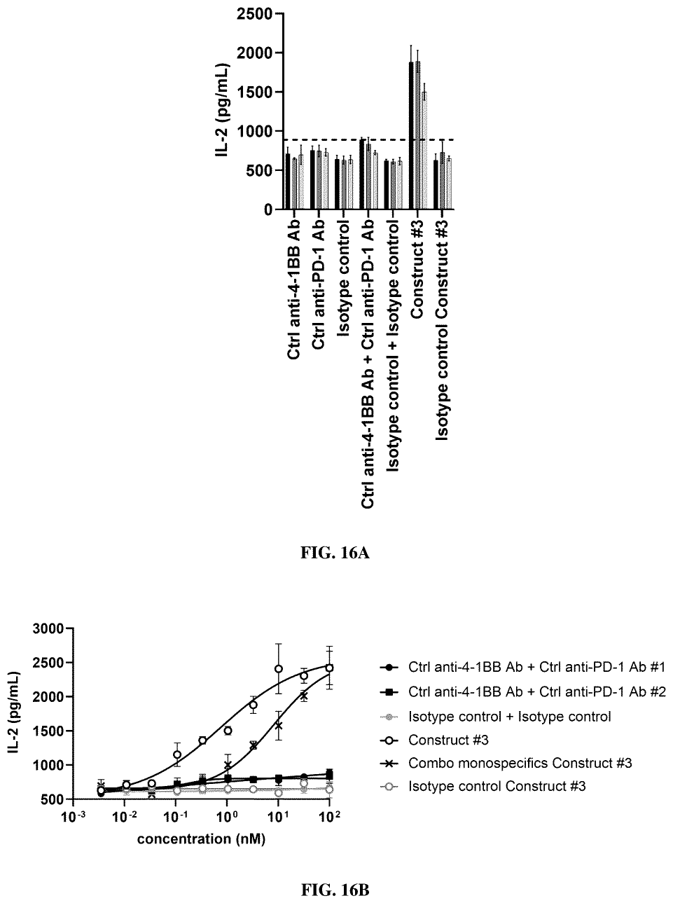

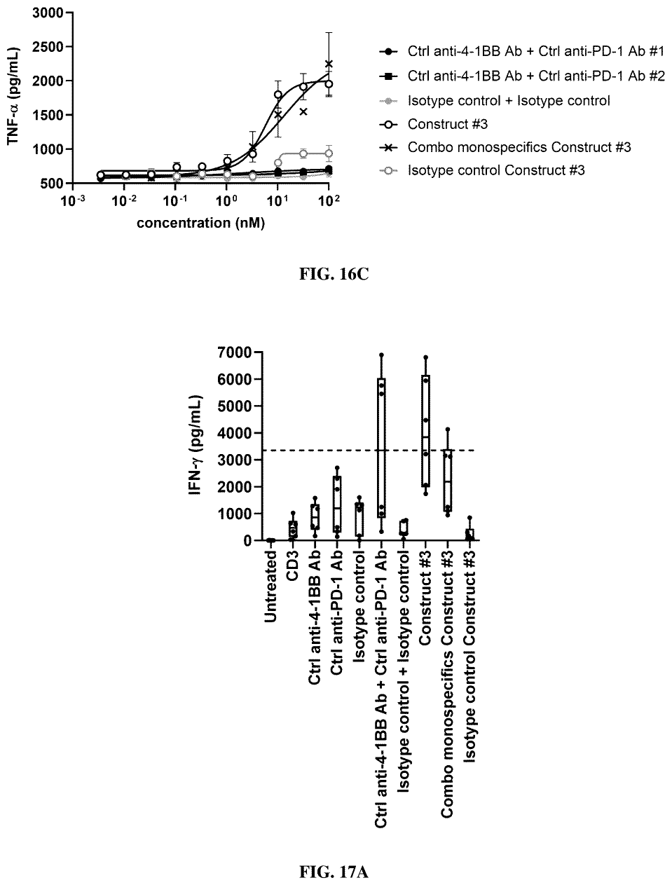

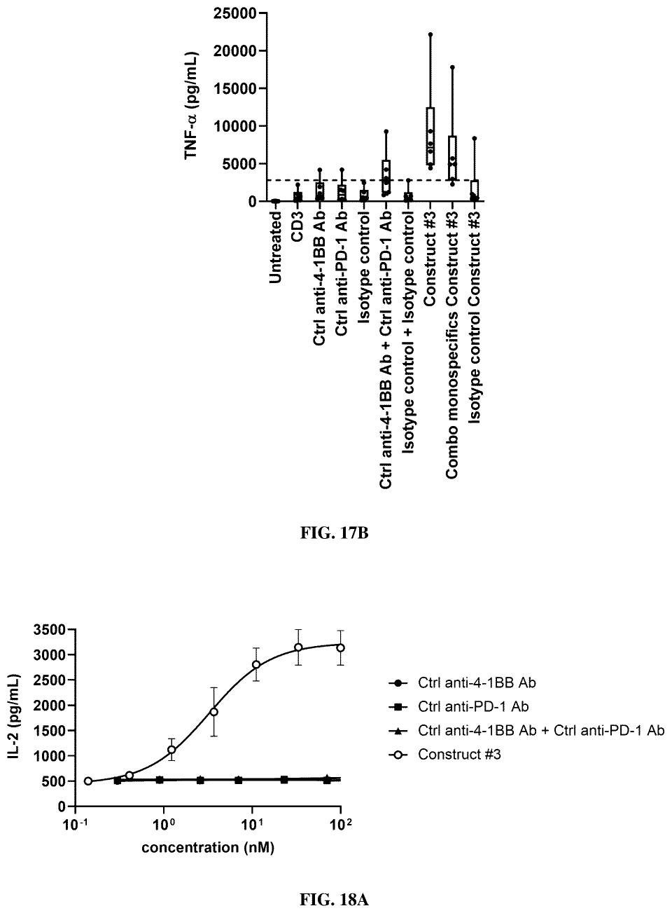

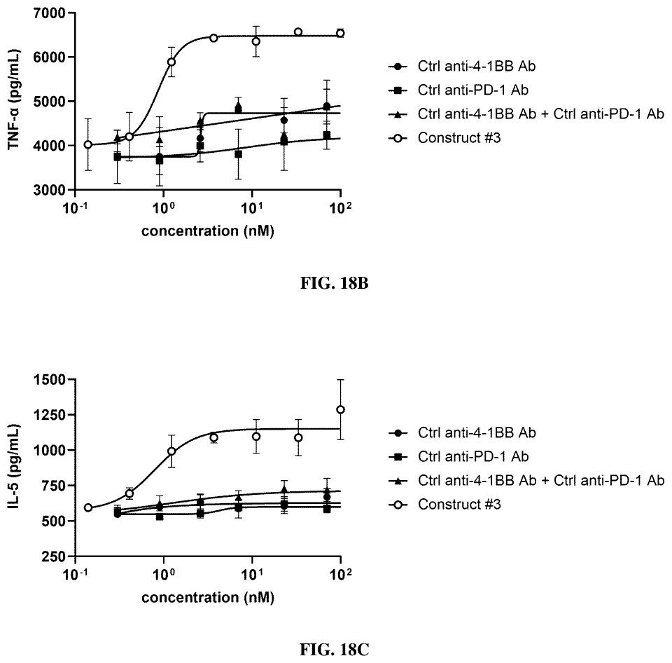

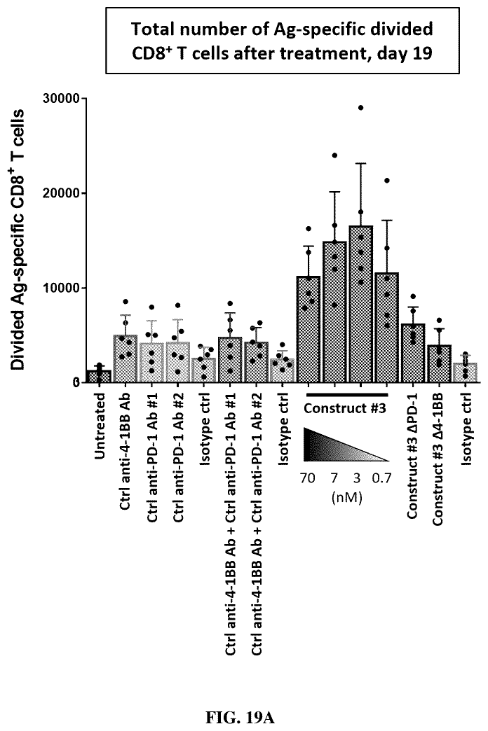

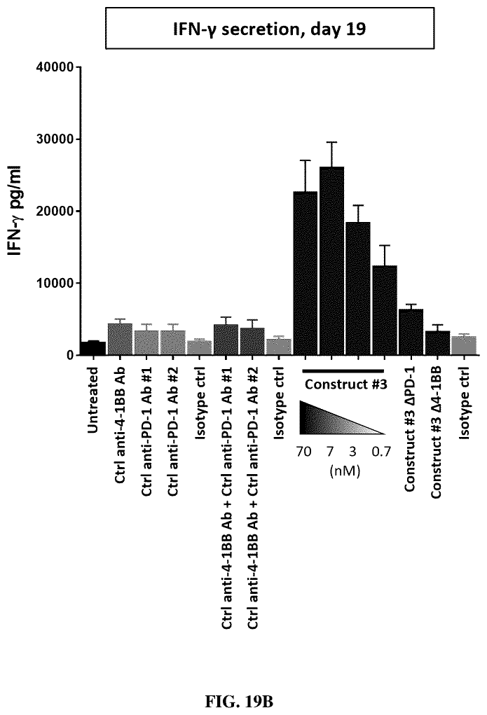

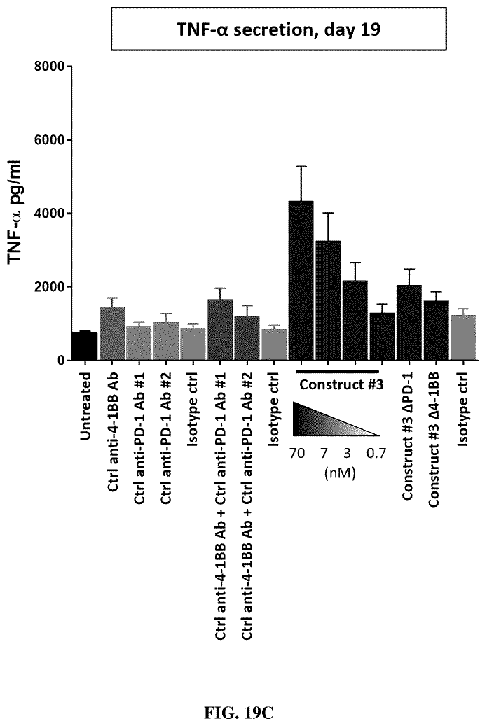

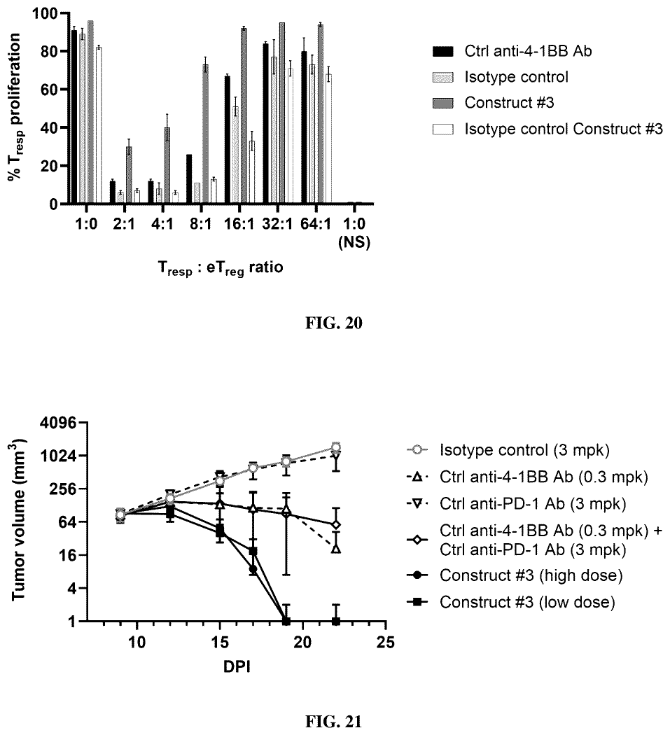

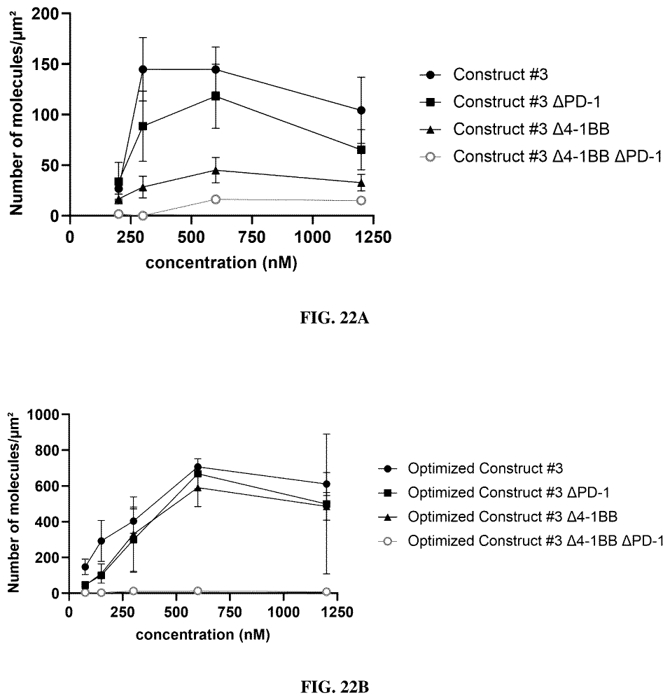

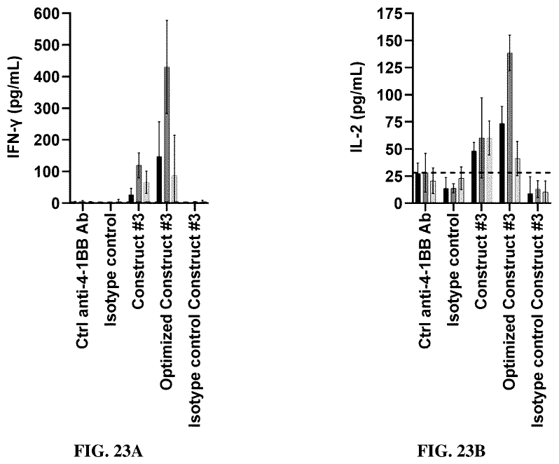

The results are shown in FIG. 2: the seven V_{HH}s showed binding to human 4-1BB and varying levels of cross-reactivity with cyno 4-1BB. None of the seven V_{HH}s showed binding to human OX40 or human CD40.

### Competition and Epitope Binning

The diversity of binding sites to 4-1BB of the seven V_{HH}s (in bivalent monoparatopic “Fab-like” constructs each comprising twice a same anti-4-1BB V_{HH}) was evaluated in competition assays against the natural ligand of 4-1BB, 4-1BBL, and against a control anti-4-1BB antibody.

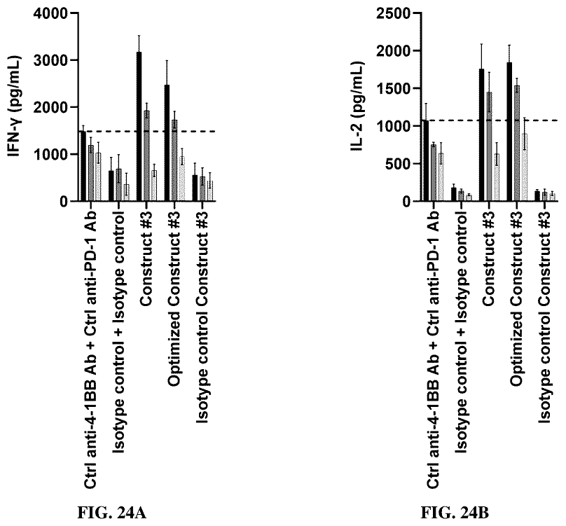

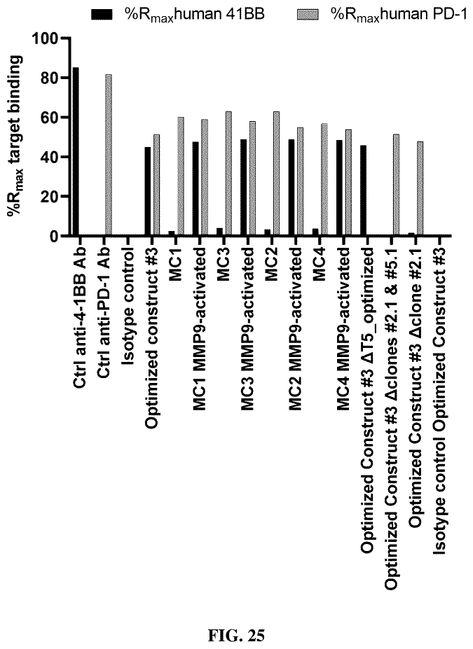

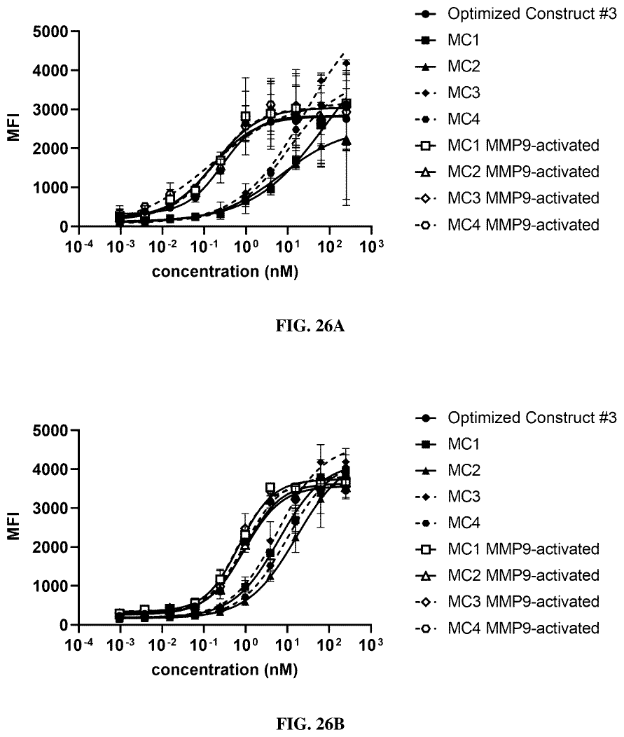

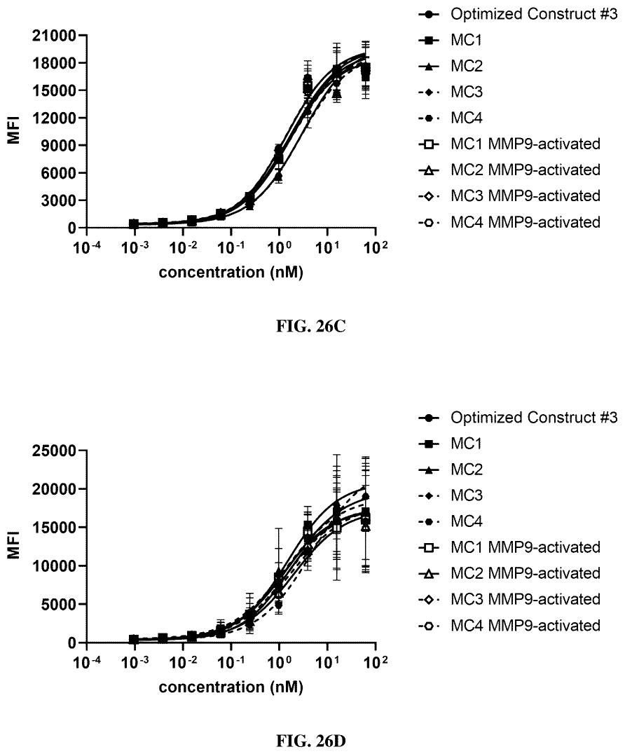

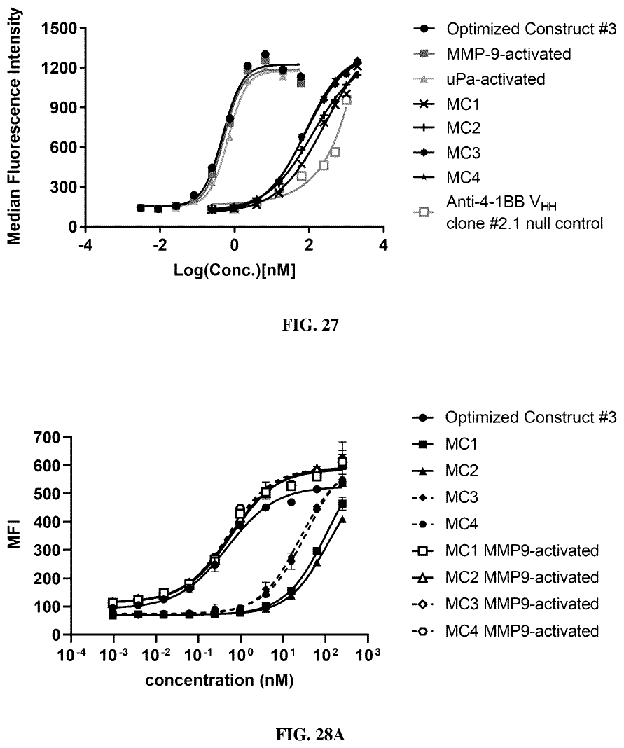

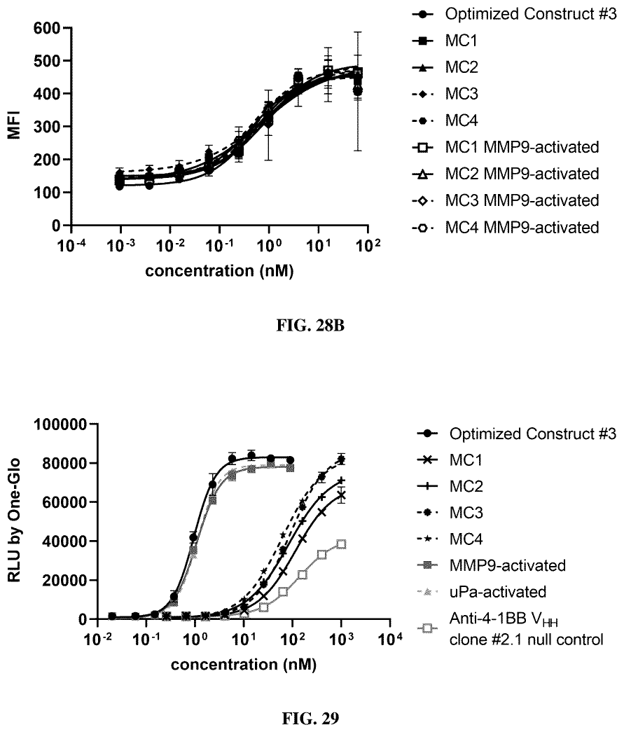

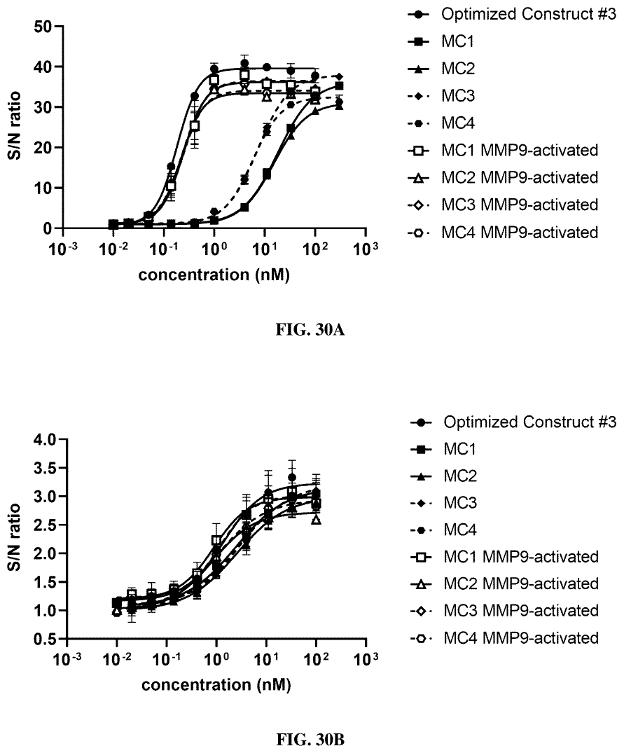

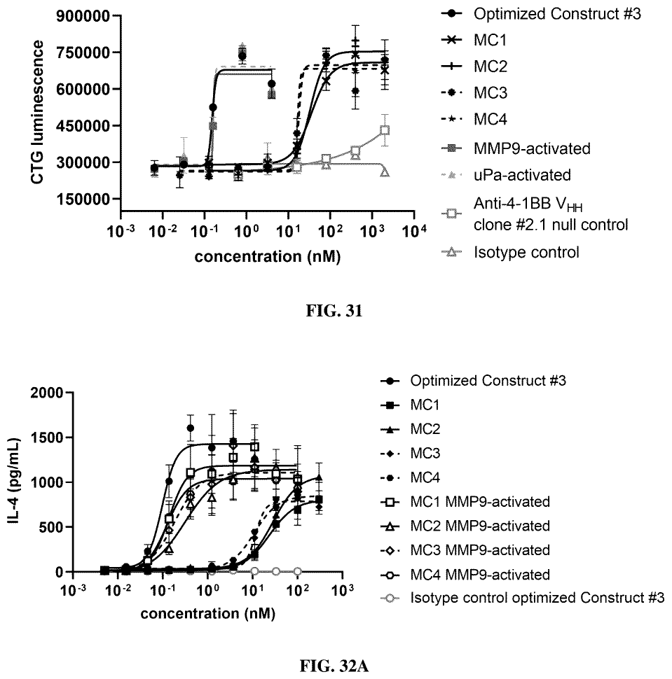

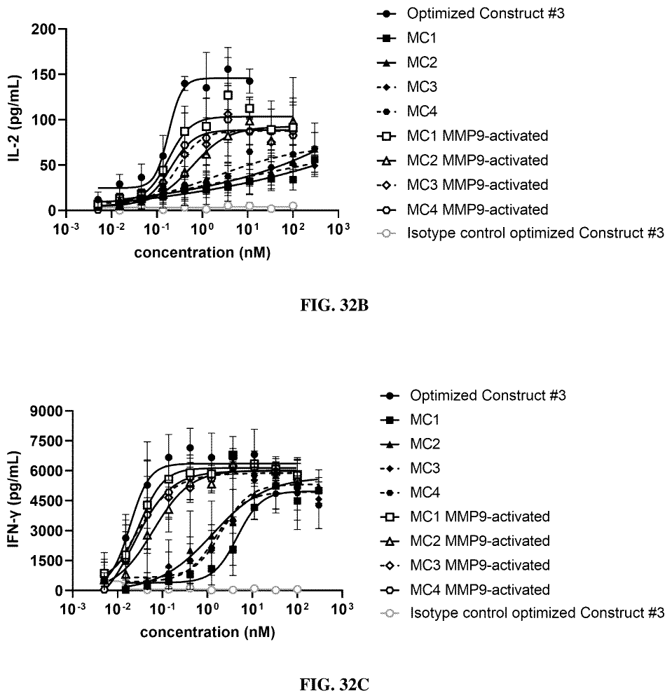

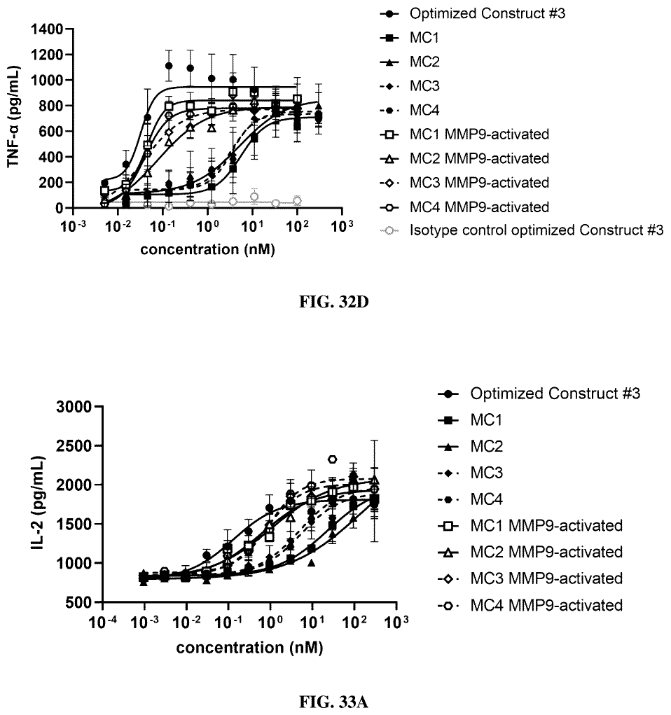

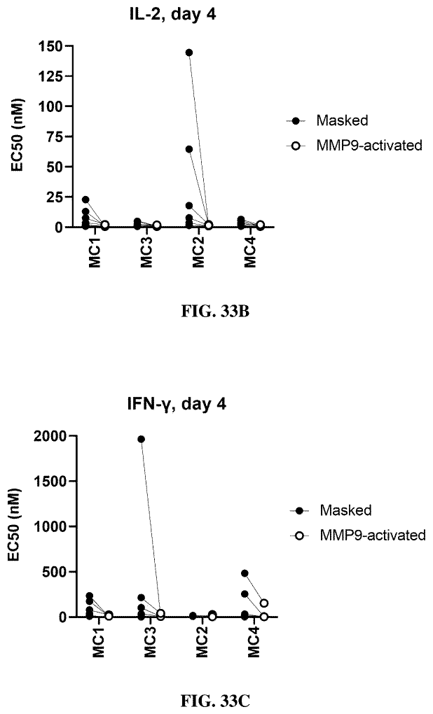

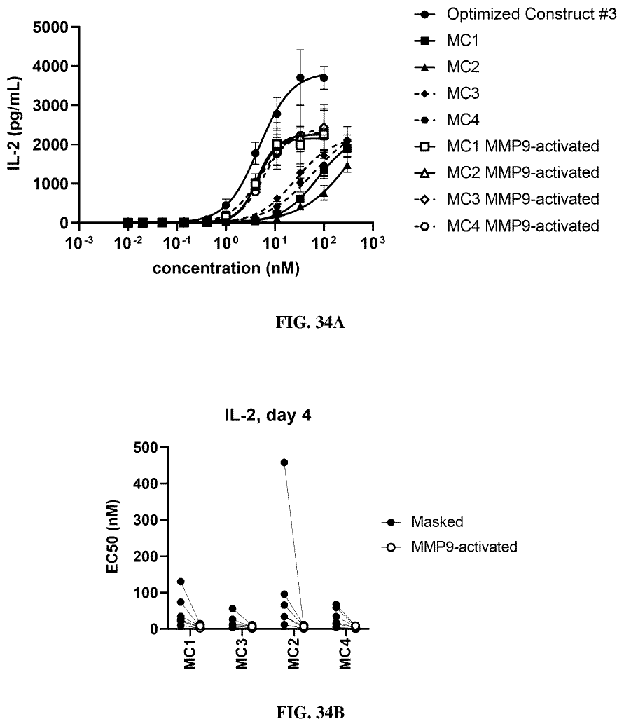

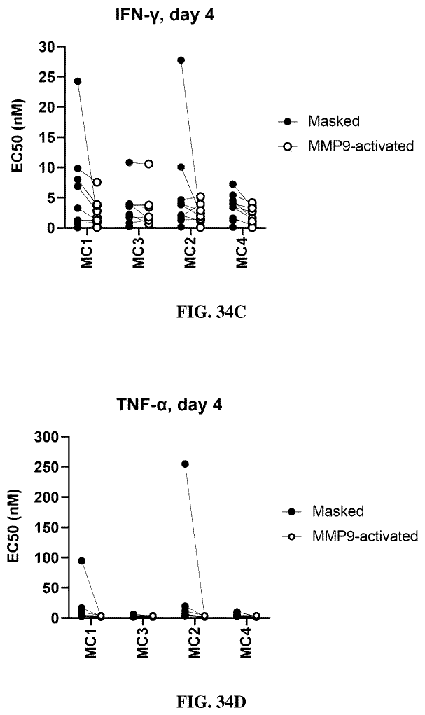

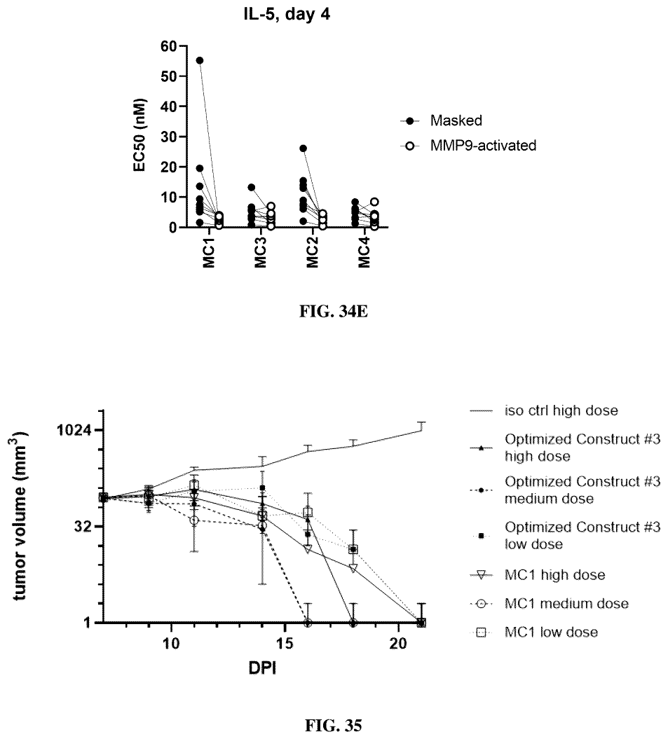

These assays allowed to identify 3 different epitope bins (bin A, bin B and bin C), according to the competition profile of the seven V_{HH}s with 4-1BBL and the anti-4-1BB antibody. The results are shown in Table 3 and FIG. 3.

TABLE 3
epitope binning

| | | Competes | Competes with | |
|---|---|---|---|---|
| | Epitope | with | control anti-4-1BB | |
| V_{HH} | binning | 4-1BBL? | antibody? | V_{HH} origin |
| Clone #1 | Bin A | No | Yes | Llama immune |
| Clone #2 | Bin A | No | Yes | library |
| Clone #3 | Bin B | No | No | |
| Clone #4 | Bin B | No | No | Naïve library |
| Clone #5 | Bin C | Yes | No | |
| Clone #6 | Bin A | No | Yes | |
| Clone #7 | Bin A | No | Yes | |

### T-Cell Activation by Anti-4-1BB V_{HH}s

Activation of 4-1BB results in activation of the NF-κB pathway. An NF-κB reporter assay was performed to detect activation of the 4-1BB signaling pathway mediated by the anti-4-1BB V_{HH}s. NF-κB activation was measured using a bioluminescent cell-based reporter assay (Promega). This assay consists of a genetically engineered Jurkat T-cell line that constitutively expresses 4-1BB and in which luciferase is controlled by NF-κB responsive elements. Thus, 4-1BB activation results in luciferase expression.

The anti-4-1BB V_{HH}s were evaluated in “Fab-like” format described previously, either in a monoparatopic bivalent form (constructs each comprising twice a same anti-4-1BB V_{HH}) or in a monoparatopic monovalent form (constructs comprising one anti-4-1BB V_{HH} and one V_{HH} directed to an irrelevant target, namely a FMDV protein), in the presence of a cross-linker reagent (anti-Fab antibody) or not (soluble condition). The results are shown in FIGS. 4A-D. All bivalent constructs (except for the negative control [anti-FMDV]) were active in a dose-dependent manner when crosslinked (FIG. 4A), but only clone #5 retained high activation potential in absence of cross-linking (FIG. 4B). The crosslinked monovalent constructs showed reduced activation potential compared to the bivalent molecules, ranging from no activation to about 25% of their respective bivalent constructs (FIG. 4C). Finally, monovalent molecules without cross-linking showed no activation in the NF-κB reporter assay (FIG. 4D).

Next, bivalent biparatopic constructs (“Fab-like” constructs each comprising two different anti-4-1BB V_{HH}) were investigated. These molecules were tested in soluble condition (i.e., in absence of cross-linking) and compared to the bivalent monoparatopic and monovalent constructs. The results are shown in FIG. 4E. Bivalent biparatopic constructs comprising clone #5 demonstrated the highest activation in the presence of any other anti-4-1BB V_{HH} tested, in soluble condition.

Altogether, these results demonstrate that a bivalent monoparatopic “Fab-like” construct comprising two anti-4-1BB clone #5 V_{HH}s is highly active in soluble condition (i.e., without cross-linking reagent such as an anti-Fab antibody, making it a “pure agonist”), and competes with 4-1BBL but not with the control anti-4-1BB antibody. Interestingly, clone #5 was shown not to be active when monovalent (combined with an irrelevant V_{HH} such as, e.g., an anti-FMDV V_{HH}); clone #5 was however active in bispecific format when combined with a V_{HH} targeting another antigen expressed by the same cell (e.g., CD28—FIG. 4F). When in bivalent biparatopic format (with another anti-4-1BB V_{HH} targeting another 4-1BB epitope), clone #5 showed an even improved strong agonistic effect of activation of the NF-κB pathway.

Finally, as shown in FIGS. 5A-B, a bispecific “Fab-like” construct comprising the anti-4-1BB clone #5 V_{HH} combined with a V_{HH} targeting an antigen expressed by another cell (e.g., OX40 expressed on an auxiliary cell) turned the anti-4-1BB clone #5 V_{HH} into a cell engager and T-cell activator, where simultaneous binding of 4-1BB on Jurkat T-cells and of the antigen (e.g., OX40) on the auxiliary cell induced immune cell activation. This feature presents interesting perspective, e.g., for targeting T-cells to pathogenic cells.

### Structural Characterization

The 3-D structure of the anti-4-1BB clone #2 and clone #5 V_{HH}s, in complex with the extracellular domain of human 4-1BB (amino acid residues 24-186 of SEQ ID NO: 13), was solved by X-ray crystallography.

The 3-D structure (FIG. 6) highlighted that both V_{HH} clones bind to different portions of 4-1BB:
the anti-4-1BB clone #2 V_{HH} binds predominantly to the first cysteine-rich domain (CRD) of 4-1BB;
the anti-4-1BB clone #5 V_{HH} binds to both the second and third CRDs of 4-1BB and overlaps with 4-1BBL binding epitope.

All three CDR1-3 of the anti-4-1BB clone #2 V_{HH} were found to interact with 4-1BB; however, of all CDRs, only CDR3 of the anti-4-1BB clone #5 V_{HH} was shown to interact with 4-1BB according to the X-ray crystallography 3-D structure.

### Anti-4-1BB Clone #5 V_{HH}s Variants

A few V_{HH}s with sequence similarities to the anti-4-1BB clone #5 V_{HH} were identified among all 4-1BB binders initially isolated. These were also produced in a bivalent monoparatopic “Fab-like” format as described above. Their binding and functional properties were evaluated: anti-4-1BB clones #5a, #5b, #5c and #5d V_{HH}s showed similar if not improved binding EC₅₀ to 4-1BB compared to the anti-4-1BB clone #5 V_{HH} (FIG. 7A), and a high activity in soluble condition (i.e., without cross-linking reagent such as an anti-Fab antibody) comparable to that of the anti-4-1BB clone #5 V_{HH} (FIG. 7B).

### Sequences

The sequences of the anti-4-1BB clone #2 and clone #5 V_{HH}s, and of variants of the anti-4-1BB clone #5 V_{HH} (clones #5a, #5b, #5c and #5d) are indicated below, with their CDR sequences highlighted (IMGT numbering in bold; Kabat numbering underlined; Chothia numbering italicized).

Clone #2 (SEQ ID NO: 1):
QVQLQESGGGLVQPGGSLRLSCAAS [symbol] TGG
WYRQAPGKQRELVATI [symbol] TNYAESVKGRFTLS
RDNAKNTVYLQMNSLKPEDTAVYYCRL [symbol]
WGQGTQVTVSS
Clone #5 (SEQ ID NO: 2):
QVQLQESGGGLVQPGGSLRLSCAAS [symbol] TMT
WVRQAPGKGLEWVSSI [symbol] IIYADSVKGRFTI
SRDNAKNTLYLQMNNLRPEDTAVYFCAR [symbol]
SGPGTQVTVSS
Clone #5a (SEQ ID NO: 58):
QVQLQESGGGWVQPGGSLRLSCAASGFAFR [symbol] TMS
WARQAPGERFEWISSI [symbol] QSYLPSVKGRFTI
SRDNAKNTMFLQMDNLTPEDTAVYFCAR [symbol]
SGPGTQVTVSS
Clone #5b (SEQ ID NO: 59):
QVQLQESGGGWVQPGGSLRLSCAAS [symbol] TMS
WARQAPGERFEWISSI [symbol] QSYLPSVKGRFTI
SRDNAKNTMFLQMDNLTPEDTAVYFCAR [symbol]
SGSGTQVTVSS
Clone #5c (SEQ ID NO: 60):
QVQLQESGGGLVQAGGSLRLSCAAS [symbol] AMG
WFRQAPGERLEWVASI [symbol] QSYHPSVKDRFTI
SRDNGKNILFLQLDKLNPEDTAVYVCAR [symbol]
SGQGTQVTVSS
Clone #5d (SEQ ID NO: 61):
QVQLQESGGGLVQPGGSLRLSCVAS [symbol] RMS
WVRQAPGKGLEWVSSI [symbol] TTYSDSVKGRFTI
SRDNAKNTMFLQMDNLTPEDTAVYFCAR [symbol]
SGPGTQVTVSS

### Example 2

### Anti-PD-1 Antibodies

We aimed at generating antagonist monoclonal anti-PD-1 antibodies binding to human and cyno PD-1, and competing with PD-1 ligand, PD-L1.

In brief, following Trianni® mice immunization, 3 556 IgG were obtained and screened for binding to human and cyno PD-1. Redundant sequences were eliminated, and the remaining IgGs were reformatted into IgG1 LALA. Hits were characterized and 59 clones were selected for further in vitro functional assays (including a PD-1/PD-L1 blockade bioassay and an allogenic MLR assay). Eight functional clones were selected after these in vitro assays; they were reformatted and produced in a Fab format to select anti-PD-1 antibodies active in monomeric format.

Ultimately, we retained one anti-PD-1 antibody, hereafter named clone “T5”, which had a human germinality index (i.e., a percentage of amino acid sequence identity in the framework regions compared to the closest human V_{H} and V_{L} germline) above 95% for both variable regions (Table 4). As evidenced in a PD-1/PD-L1 interaction assay, anti-PD-1 antibody clone T5 was also active in a monomeric Fab format (Table 5).

TABLE 4

| | Heavy chain | | Light chain | |
|---|---|---|---|---|
| | Closest | Germi- | Closest | Germi- |
| | human | nality | human | nality |
| | V_{H} germline | index (%) | V_{H} germline | index (%) |
| Clone T5 | IGHV4-39 | 96.97 | IGKV3D-20 | 95.83 |
| Control anti-PD-1 | IGHV3-33 | 90.82 | IGKV3-11 | 98.95 |
| antibody #1 | | | | |
| Control anti-PD-1 | IGHV1-2 | 79.59 | IGKV3-20 | 84.95 |
| antibody #2 | | | | |

TABLE 5

| | Relative EC₅₀ (M) | | Ratio |
|---|---|---|---|
| | IgG1 LALA | Fab | Fab:IgG1 |
| Clone T5 | 2.78 × 10⁻¹⁰ | 4.88 × 10⁻⁸ | 176 |
| Control anti-PD-1 | 1.81 × 10⁻¹⁰ | >1.00 × 10⁻⁶ | >1.00 × 10⁻⁴ |
| antibody #1 | | | |
| Control anti-PD-1 | 4.95 × 10⁻¹⁰ | 1.48 × 10⁻⁸ | 30 |
| antibody #2 | | | |

### Sequences

The anti-PD-1 clone T5 has a light chain variable region (LCVR) with the amino acid sequence set forth in SEQ ID NO: 5:

(SEQ ID NO: 5)
EIVLTQSPATLSLSPGERATLSCGAS [symbol] LAW
YQQKPGLAPRLLIY [symbol] SRATGIPDRFSGSGSGTDF
TLTISRLEPEDFAVYYC [symbol] FGQGTKLEIK

CDR sequences are highlighted: IMGT numbering in bold; Kabat numbering underlined; Chothia numbering italicized.

The anti-PD-1 clone T5 has a heavy chain variable region (HCVR) with the amino acid sequence set forth in SEQ ID NO: 6:

(SEQ ID NO: 6)
QLQLQESGPGLVKPSETLSLTCTVS [symbol] FWG
WIRQPPGKGLEWIGSI [symbol] TYYNPSLKSRVTISVD
TSKNQFSLKLSSVTAADTAVYYCAR [symbol]
YWGQGTLVTVSS

CDR sequences are highlighted: IMGT numbering in bold; Kabat numbering underlined; Chothia numbering italicized.

### Example 3

### Anti-4-1BB/Anti-PD-1 Bispecific Binding Proteins

The Inventors aimed at developing an anti-4-1BB/anti-PD-1 bispecific binding molecule, comprising a pure agonist anti-4-1BB V_{HH} as described in Example 1 (clone #5) and a proprietary anti-PD-1 binding protein as described in Example 2 (clone “T5”).

Several constructs were designed and tested, comprising from 1 to 2 anti-PD-1 Fabs (+a Fc region) and from 3 to 6 anti-4-1BB V_{HH}s, in various orientations. Out of these constructs, four bispecific constructs were initially selected (FIG. 8).

Construct #1 comprises, from N- to C-terminal:
the anti-PD-1 clone T5 Fab;
an IgG1-LALA* Fc region;
a variant of the anti-4-1BB clone #2 V_{HH} described in Example 1 (namely, clone #2.1); and
a variant of the anti-4-1BB clone #5 V_{HH} described in Example 1 (namely, clone #5.1).

Constructs #3, #5 and #6 comprise, from N- to C-terminal:
a variant of the anti-4-1BB clone #2 V_{HH} described in Example 1 (namely, clone #2.1);
a variant of the anti-4-1BB clone #5 V_{HH} described in Example 1 (namely, clone #5.1);
an IgG1-LALA* Fc region (Construct #3), an IgG1-NNAS** Fc region (Construct #5), or an IgG4-P-FALA*** Fc region (Construct #6); and
an inverted anti-PD-1 clone T5 Fab.
   *LALA refers to an ADCC-silencing mutation (L234A/L235A) impairing IgG1 Fc effector functions.
   **NNAS refers to an ADCC-silencing mutation (S298N/T299A/Y300S) abrogating IgG1 Fc effector functions.
   ***P-FALA refers to an ADCC-silencing mutation (S228P/F234A/L235A) impairing IgG4 Fc effector functions.

### Material and Methods

### Expression and Purification

### HEK293-FS Cells

The expression plasmids encoding the heavy and light chains of the test compounds were propagated in E. coli DH5a. Plasmids used for transfection were prepared from E. coli using the EndoFree® Plasmid Mega kit (Qiagen, ref. 12381).

HEK293-FS cells growing in F17 serum-free suspension culture (Invitrogen) were transfected with heavy and light chain plasmids using polyethylenimine (PEI) transfection reagent. After 7 days of cultivation at 37° C., cells were removed by centrifugation and the supernatant was passed over a 0.22-μm filter to remove particles.

For purification, the test compounds were captured on a HiTrap® MabSelect SuRe™ column (GE Healthcare, Ref. 11-0034-93), eluted with 0.1 M citrate buffer pH 3.0, and directly desalted using a HiPrep 26/10 desalting column (GE Healthcare, Ref. 17-05087-02). After polishing the protein by size exclusion chromatography using a HiLoad® 26/600 Superdex® 200 (GE Healthcare, Ref. 28-9893-36) and a final ultrafiltration concentration step, the test compounds were used for further characterization.

### CHO Cells

The expression plasmids encoding the heavy and light chains of the test compounds were cloned into Evitria's (Zurich, Switzerland) proprietary expression vector system using conventional, non-PCR based, cloning techniques. The expression vectors were gene-synthesized. Plasmid DNA was prepared under low-endotoxin conditions based on anion exchange chromatography. DNA concentration was determined by measuring the absorption at a wavelength of 260 nm. Correctness of the sequences was verified with Sanger sequencing (with up to two sequencing reactions per plasmid depending on the size of the cDNA).

Suspension-adapted CHO K1 cells were used for production. The seed was grown in eviGrow medium (Evitria), a chemically defined, animal-component free, serum-free medium. Cells were transfected with eviFect (Evitria), a custom-made transfection reagent, and cells were grown after transfection in eviMake2 (Evitria), an animal-component free, serum-free medium.

Supernatant was harvested by centrifugation and subsequent filtration on a 0.2-μm filter.

The test compounds were purified using a HiTrap® MabSelect SuRe™ column (GE Healthcare, Ref. 11-0034-93) and a HiLoad® 26/600 Superdex® 200 (GE Healthcare, Ref. 28-9893-36), using the same protocol as for HEK293-FS purification described above.

### Binding Assay on Stable Cell Lines

### Human PD-1 and 4-1BB

Goal: evaluate the affinity of the anti-4-1BB/anti-PD-1 bispecific binding molecule versus control antibodies and isotype controls on their targets (4-1BB and PD-1 arms separately). Calculate the binding EC₅₀ and the Eₘₐₓ values of the test compounds.

The binding assay was performed in 96-well plates on ice using PD-1 NFAT-luc2 Jurkat cells (Promega #J1252), or 4-1BB NF-κB-luc2P Jurkat cells (Promega #J2332), or in-house transfected 300.19 (pre-B) cells expressing either human PD-1 (hPD-1) or human 4-1BB (h4-1BB).

A cell suspension was plated at a density of 50×10³ cells per well in 96-well U bottom plates. Serially diluted concentrations of test compounds were added to the cells for 1 hour. After washing, a fluorescently labeled secondary antibody targeting the Fc portion was added to each well for 30 minutes. The MFI signal was then measured using a flow cytometer.

Data from the flow cytometer was analyzed using FlowJo (V10.8.1), then the binding curves, the Eₘₐₓ, and EC₅₀ values were plotted using GraphPad Prism (V9.1.2).

### Cynomolgus Monkey PD-1 and 4-1BB

Goal: evaluate the affinity of the anti-4-1BB/anti-PD-1 bispecific binding molecule versus control antibodies and isotype controls on their targets (4-1BB and PD-1 arms separately). Calculate the binding EC₅₀ and the Eₘₐₓ values of the test compounds.

The binding assay was performed in 96-well plates on ice using in-house transfected 300.19 (pre-B) cells expressing either cyno PD-1 (cyPD-1) or cyno 4-1BB (cy4-1BB).

A cell suspension was plated at a density of 50×10³ cells per well in 96-well U bottom plates. Serially diluted concentrations of test compounds were added to the cells for 1 hour. After washing, a fluorescently labeled secondary antibody targeting the Fc portion was added to each well for 30 minutes. The MFI signal was then measured using a flow cytometer.

Data from the flow cytometer was analyzed using FlowJo (V10.8.1), then the binding curves, the Eₘₐₓ, and EC₅₀ values were plotted using GraphPad Prism (V9.1.2).

### Binding Assay on Primary Human T-Cells

Goal: evaluate the affinity of the anti-4-1BB/anti-PD-1 bispecific binding molecule versus control antibodies and isotype controls on their targets (4-1BB and PD-1 simultaneously). Calculate the binding EC₅₀ and the Eₘₐₓ values of the test compounds.

Human PBMC were isolated from fresh buffy coats using a Ficoll gradient. Total CD3⁺ T-cells were isolated via negative selection. T-cells were plated at a density of 50×10³ cells/well in 96-well U-bottom culture plates previously coated with 5 μg/mL of anti-human CD3 and cultured in X-Vivo 15 media (Lonza #BE02-061Q) supplemented with 1% penicillin/streptomycin.

Serially diluted concentrations of test compounds were added to the cells for 1 hour. After washing, a fluorescently labeled secondary antibody targeting the Fc portion was added to each well for 30 minutes. The MFI signal was then measured using a flow cytometer.

Data from the flow cytometer was analyzed using FlowJo (V10.8.1), then the binding curves, the Eₘₐₓ, and EC₅₀ values were plotted using GraphPad Prism (V9.1.2).

### Reporter Assays

### 4-1BB

Goal: Assess the 4-1BB arm activity of the anti-4-1BB/anti-PD-1 bispecific binding molecule versus control antibodies and isotype controls. Calculate the EC₅₀ and the Eₘₐₓ for each of the test compounds.

Readout using the 4-1BB reporter Jurkat NF-κB-luc2P Promega cell line (GloResponse NF-κB-luc2P Jurkat cells, Promega #J2332) cultured in RPMI 1640, 10% SVF, 1% glutamine, 1% NEAA, 1 mM sodium pyruvate, 800 μg/mL G418, 500 μg/mL hygromycin following the manufacturer's instructions.

Cells were plated at a density of 50×10³ cells per well in 96-well white flat bottom plates. Serially diluted concentrations of test compounds were added. After an incubation of 6 hours at 37° C., Bio-Glo Reagent was added to each well. Luminescence was measured using an Infinit Pro M1000 or a SPARK TECAN reader.

The binding curves, Eₘₐₓ, and EC₅₀ values were plotted using GraphPad Prism (V9.1.2).

### PD-1

Goal: Assess the PD-1 arm activity of the anti-4-1BB/anti-PD-1 bispecific binding molecule versus control antibodies and isotype controls. Calculate the IC₅₀ and the Imax for each of the test compounds.

Readout using the PD-1 reporter Jurkat NFAT-luc2 Promega cell line (GloResponse PD-1 NFAT-luc2 Jurkat cells, Promega #J1252) cultured in RPMI 1640, 10% SVF, 1% glutamine, 1% NEAA, 1 mM PyNa, 500 μg/mL G418, 200 μg/mL hygromycin in presence of the PD-L1 aAPC/CHO-K1 accessory cells (Promega #J1252) cultured in HAM/F12, 10% SVF, 200 μg/mL hygromycin, 250 μg/mL G418, following the manufacturer's instructions.

PD-L1 aAPC/CHO-K1 were plated at a density of 40×10³ cells per well in 96-wells white flat bottom culture plates and were incubated overnight at 37° C. in Ham/F12 media supplemented with 1% FCS. The following day, media was removed, a PD-1 NFAT-luc2 Jurkat cells suspension at a density of 50×10³ cells/well was added in the wells, and serially diluted concentrations of test compounds were added. After an incubation of 6 hours at 37° C., Bio-Glo Reagent was added to each well. Luminescence was measured using an Infinit Pro M1000 or a SPARK TECAN reader.

The binding curves, Imax, and IC₅₀ values were plotted using GraphPad Prism (V9.1.2).

### ADCC ADCP CDC Assays

### Antibody-Dependent Cellular Cytotoxicity (ADCC) Assay

Goal: evaluate the ADCC effect of the anti-4-1BB/anti-PD-1 bispecific binding molecule versus control antibodies and isotype controls in dose-response experiments. Calculate the EC50 and the Eₘₐₓ values of the test compounds.

For the ADCC assay, NFAT-luc2 Jurkat reporter cells overexpressing the human FcγRIIIa-V158 high sensitivity variant were used as effector cells (Promega #G7102) in co-culture with in-house-generated murine 300.19 (pre-B) target cells expressing either human PD-1 or human 4-1BB. 1.5×10⁵ Jurkat effector cells and 7.5×10⁴ 300.19 target cells (ET ratio=2:1) per well were plated in 96-well U bottom suspension plates together with test compounds in triplicate wells from 200 nM to 0.003 nM concentration in nine serial 1:4 dilution steps. Incubation was performed for 24 hours at 37ºC, 5% CO₂ and 95% rH, followed by readout using the Bio-Glo Luciferase Assay System (Promega #G7940) and a Tecan Spark luminescence microplate reader.

EC₅₀ and Eₘₐₓ values of the test compounds were calculated by applying a Biostat-Speed statistical calculation tool. Results were obtained using the 4-parameter logistic model according to Ratkovsky & Reedy (Biometrics. 1986 September;42(3):575-82). The adjustment was obtained by non-linear regression using the Levenberg-Marquardt algorithm in SAS v9.1.3 software.

### Antibody-Dependent Cellular Phagocytosis (ADCP) Assay

Goal: evaluate the ADCP effect of the anti-4-1BB/anti-PD-1 bispecific binding molecule versus control antibodies and isotype controls in dose-response experiments. Calculate the EC₅₀ and the Eₘₐₓ values of the test compounds.

For the ADCP assay, NFAT-luc2 Jurkat reporter cells overexpressing the human FcγRIIa-H131 high sensitivity variant were used as effector cells (Promega #G9871) in co-culture with in-house-generated murine 300.19 (pre-B) target cells expressing either human PD-1 or human 4-1BB. 3×10⁴ Jurkat effector cells and 1.5×10⁴ 300.19 4-1BB target cells (E:T ratio=2:1) or 105 Jurkat effector cells and 10⁵ 300.19 PD-1 target cells (E:T ratio=1:1) per well were plated in 96-well U bottom suspension plates together with test compounds in triplicate wells from 200 nM to 0.003 nM concentration in nine serial 1:4 dilution steps. Incubation was performed for 24 hours at 37° C., 5% CO₂ and 95% rH, followed by readout using the Bio-Glo Luciferase Assay System (Promega #G7940) and a Tecan Spark luminescence microplate reader.

EC₅₀ and Eₘₐₓ values of the test compounds were calculated by applying a Biostat-Speed statistical calculation tool. Results were obtained using the 4-parameter logistic model according to Ratkovsky & Reedy (Biometrics. 1986 September;42(3):575-82). The adjustment was obtained by non-linear regression using the Levenberg-Marquardt algorithm in SAS v9.1.3 software.

### Complement-Dependent Cytotoxicity (CDC) Assay

Goal: evaluate the CDC effect of the anti-4-1BB/anti-PD-1 bispecific binding molecule versus control antibodies and isotype controls in dose-response experiments. Calculate the IC₅₀ and the Imax values of the test compounds.

For the CDC assay, 1.5×10⁴ in-house generated murine 300.19 (pre-B) target cells expressing either human PD-1 or human 4-1BB were plated per well in 96-well U bottom suspension plates together with 10% human serum complement (Quidel #A113) and test compounds in triplicate wells from 200 nM to 0.003 nM concentration in nine serial 1:4 dilution steps. Incubation was performed for 24 hours at 37ºC, 5% CO₂ and 95% rH, followed by readout using the CellTiter-Glo Luminescent Cell Viability Assay System (Promega #G7571) and a Tecan Spark luminescence microplate reader.

IC₅₀ and the Imax values of the test compounds were calculated by applying a Biostat-Speed statistical calculation tool. Results were obtained using the 4-parameter logistic model according to Ratkovsky & Reedy (Biometrics. 1986 September;42(3):575-82). The adjustment was obtained by non-linear regression using the Levenberg-Marquardt algorithm in SAS v9.1.3 software.

### T-Cell Activation (TCA)

### Human

Goal: Assess the activity of the anti-4-1BB/anti-PD-1 bispecific binding molecule versus control antibodies and isotype controls. Observe the cytokine release from activated human T-cells.

PBMC were isolated from fresh buffy coats using a Ficoll gradient. T-cells were isolated via a negative selection. T-cells were plated at a density of 100×10³ cells/well in 96-flat bottom well plates previously coated with 5 μg/mL of anti-human CD3 and cultured in X-Vivo 15 media (Lonza #BE02-061Q) supplemented with 1% penicillin/streptomycin.

Serially diluted concentrations of test compounds were added to the culture. After 4 or 6 days of incubation, the cytokines secretion was evaluated in the harvested supernatants either with a CBA human Th1/Th2 cytokines kit on a flow cytometer according to the manufacturer's instructions (BD Bioscience #550749), or with a Homogeneous Time Resolved Fluorescence (HTRF) human IFN-γ/TNF-α cytokines kit (Cisbio #62HIFNGPEH and #62HTNFAPEH) on a PHERAstar FSX multimode reader (BMG Labtech) according to the manufacturer's instructions.

The cytokines secretion was analyzed with FCAP array (V3.0.19.2091) for CBA samples, then plotted using GraphPad Prism (V9.1.2). HTRF cytokines secretion was directly plotted using GraphPad Prism (V9.1.2).

### Cynomolgus Monkey

Goal: Assess the activity of the anti-4-1BB/anti-PD-1 bispecific binding molecule versus control antibodies and isotype controls. Observe the cytokine release from activated cyno T-cells.

Frozen cyno PBMC were obtained from Primacyt. Total CD3⁺ T-cells were isolated via a negative selection. T-cells were plated at a density of 50×10³ cells/well in 96-well V-bottom culture plates and cultured in X-Vivo 15 media (Lonza #BE02-061Q) supplemented with 1% penicillin/streptomycin.

Serially diluted concentrations of test compounds were added to the culture in the presence of a NHP T-cell activation/expansion kit (Miltenyi #130-092-919) at a 1:1 ratio of cells:beads. After 2 days of incubation, the cytokines secretion was evaluated in the harvested supernatants by MSD according to the manufacturer's instructions (Meso QuickPlex SQ120 #1300).

The cytokines secretion was analyzed with MSD Discovery Workbench (V4.0.12), then plotted using GraphPad Prism (V9.1.2).

### Mixed Lymphocyte Reaction (MLR) Assay

Goal: Assess the activity of the anti-4-1BB/anti-PD-1 bispecific binding molecule versus control antibodies and isotype controls in a MLR assay. Observe the cytokine release from the co-culture.

PBMC were isolated from fresh buffy coats using a Ficoll gradient. Monocytes were isolated via a positive selection. Monocytes were cultured for 6 days in presence of 50 ng/ml GM-CSF and 10 ng/mL IL-4 in RPMI 1640, 10% FCS, 2 mM glutamine, 1% penicillin/streptomycin to differentiate them into Mo-DCs. In parallel, PBMC were isolated from fresh buffy coats from another donor using a Ficoll gradient. T-cells were isolated via a negative selection.

A mixture of heterologous T-cells and Mo-DCs at a ratio 10:1 T-cells:Mo-DCs was plated in 96-well U bottom plates and cultured in X-Vivo 15 media (Lonza #BE02-061Q) supplemented with 1% penicillin/streptomycin.

Serially diluted concentrations of test compounds were added to the co-culture. After 4 or 6 days of incubation, the cytokines secretion was evaluated in the harvested supernatants either with a CBA human Th1/Th2 cytokines kit on a flow cytometer according to the manufacturer's instructions (BD Bioscience #550749), or with a Homogeneous Time Resolved Fluorescence (HTRF) human IFN-γ/TNF-α cytokines kit (Cisbio #62HIFNGPEH and #62HTNFAPEH) on a PHERAstar FSX multimode reader (BMG Labtech) according to the manufacturer's instructions.

The cytokines secretion was analyzed with FCAP array (V3.0.19.2091) for CBA samples, then plotted using GraphPad Prism (V9.1.2). HTRF cytokines secretion was directly plotted using GraphPad Prism (V9.1.2).

### CD3-PBMC Activation Assay

Goal: Assess the activity of the anti-4-1BB/anti-PD-1 bispecific binding molecule versus control antibodies and isotype controls in a CD3-PBMC activation assay. Observe the cytokine release from PBMC.

PBMC were isolated from fresh buffy coats and plated at a density of 150×10³ cells/well and cultured in presence of 0.04 μg/mL soluble anti-CD3 and serially diluted concentrations of test compounds. After 4 or 6 days of incubation, the cytokines secretion was evaluated in the harvested supernatants either with a CBA human Th1/Th2 cytokines kit on a flow cytometer according to the manufacturer's instructions (BD Bioscience #550749), or with a Homogeneous Time Resolved Fluorescence (HTRF) human IFN-γ/TNF-α cytokines kit (Cisbio #62HIFNGPEH and #62HTNFAPEH) on a PHERAstar FSX multimode reader (BMG Labtech) according to the manufacturer's instructions.

The cytokines secretion was analyzed with FCAP array (V3.0.19.2091) for CBA samples, then plotted using GraphPad Prism (V9.1.2). HTRF cytokines secretion was directly plotted using GraphPad Prism (V9.1.2).

### Modular IMmune In Vitro Construct (MIMIC) CD8⁺ T-Cells Exhaustion Assay

Goal: Assess the activity of the anti-4-1BB/anti-PD-1 bispecific binding molecule versus control antibodies and isotype controls in a CD8⁺ T-cells exhaustion assay. Observe the proliferation of exhausted CD8⁺ T-cells and their cytokines release.

Monocytes were isolated from frozen PBMC via a positive selection and cultured for 6 days in presence of 500 ng/ml GM-CSF and 125 ng/ml IL-4 in CellGro media (CellGenix) to differentiate them into cytokine-derived dendritic cells (CDDCs). CDDCs were harvested and resuspended at a density of 3.34×10⁴ cells/well in 24-well plates in X-Vivo 15 media (Lonza #BE02-061Q). CDDCs were primed for 2-3 hours with HLA-restricted peptides (BioSynthesis) at 1 μg/mL.

CD8⁺ T-cells were isolated from the same autologous PBMC donors via a negative selection. CD8⁺ T-cells were plated at a density of 2×10⁶ cells/well in the plates containing pulsed CDDCs at a CD8⁺ T-cells:CDDCs ratio of 60:1 and cultured in X-Vivo 15 media (Lonza #BE02-061Q) for 12 days.

Functional CD8⁺ T-cells:CDDCs co-cultures remained untouched during the 12-day period. Exhausted CD8⁺ T-cells:CDDCs co-cultures were pulsed again at days 4 and 8 with HLA-restricted peptides at 1 μg/mL.

At day 6, autologous CDDCs were reprepared as above. At day 12, CDDCs were harvested and plated at a density of 5×10⁴ cells/well in 96-well plates in X-Vivo 15 media (Lonza #BE02-061Q). CDDCs were primed for 2-3 hours with HLA-restricted peptides at 1 μg/mL.

CD8⁺ T-cells:CDDCs co-cultures were harvested and CFSE-stained CD8⁺ T-cells were plated at a density of 5×10⁵ cells/well in the plates containing pulsed CDDCs at a CD8⁺ T-cells:CDDCs ratio of 10:1.

Serially diluted concentrations of test compounds were added to the co-culture. After 5 days of incubation, IFN-γ and TNF-α secretion was evaluated in the harvested supernatants with the Milliplex Human Cytokine Custom 10 Plex detection system (EMD Millipore) according to the manufacturer's instructions. IFN-γ and TNF-α secretion was measured on a BioPlex/Luminex system (BioRad). After washing, CFSE-stained CD8⁺ T-cells' MFI was measured by flow cytometry. Pentamer-positive CD8⁺ T-cells were detected according to the manufacturer's instructions (BioSynthesis).

The cytokines secretion was analyzed with BioPlex Manager Software, then plotted with GraphPad Prism (V9.1.2). Flow cytometry results were analyzed using FlowJo (V10.8.1), then plotted using GraphPad Prism (V9.1.2).

### Regulatory T-Cells (T_{reg}) Suppression Assay

Goal: Assess the activity of the anti-4-1BB/anti-PD-1 bispecific binding molecule versus control antibodies and isotype controls in a T_{reg} suppression assay. Observe the CD4+ effector T-cells (T_{eff}) proliferation in presence of suppressive T_{reg}.

PBMC were isolated from fresh buffy coats using a Ficoll gradient. Total CD4⁺ T-cells were isolated via a negative selection, then a CD4⁺CD25⁺ T_{reg} fraction was isolated via positive selection and expanded for 14 days in presence of CD3/CD28 MACSiBeads (Miltenyi #130-095-353) in TexMACS media (Miltenyi #130-097-196) supplemented with 5% FBS and 1% penicillin/streptomycin. The other T_{eff} CD4+CD25 fraction was frozen for later use. Readout: T_{eff} proliferation using carboxyfluorescein succinimidyl ester (CFSE) dilution following the manufacturer's instructions.

Expanded T_{reg} (eT_{reg}) were cultured with CFSE-stained T_{eff} from the same donor at different ratio (2:1, 4:1, 8:1, 16:1, 32:1, 64:1) of T_{eff}:eT_{reg} cells in presence of CD2/CD3/CD28 MACSiBeads (Miltenyi #130-092-909) at a bead:cell ratio of 1:1. Serially diluted concentrations of test compounds were added to the culture. After 5 days of incubation, the CFSE staining's MFI was measured by flow cytometry.

Flow cytometry results were analyzed using FlowJo (v.10.8.1), then plotted using GraphPad Prism (v.9.1.2).

### In Vivo Efficacy Study

C57BL/6-Pdcdl^{tm1(PDCDl)} Tnfrsf9^{tm1(TNFRSF9)}/Bcgen mice (humanized hPD-1/h4-1BB double KI) from Biocytogen (Ref. 120516) were subcutaneously implanted with MC38 tumor cells and randomized when tumors were established (≈100 mm³). Test compounds were administered directly post-randomization by i.p. route in a Q3D regimen.

### Results

As shown in FIG. 9A-9C, all four bispecific binding molecules (Constructs #1, 3, 5 and 6) were capable of activating T-cells to a greater extent than monospecific control antibodies directed to 4-1BB or PD-1 separately, but also more than the combination of the two monospecific control antibodies.

Construct #3 was selected for further experiments.

### Binding Assays on Stable Cell Lines

Table 6 summarizes the EC₅₀ and Eₘₐₓ values of “Construct #3” versus control antibodies measured in in-house transfected 300.19 (pre-B) cells overexpressing either PD-1 (human, FIG. 10A; or cyno, FIG. 10B) or 4-1BB (human, FIG. 10C or cyno, FIG. 10D).

TABLE 6
EC₅₀ and Eₘₐₓ values in 300.19 (pre-B) cells

| | | Pre-B | Pre-B | Pre-B | Pre-B |
|---|---|---|---|---|---|
| | | h4-1BB | cy4-1BB | hPD-1 | cyPD-1 |
| Construct #3 | EC₅₀ (nM) | 0.2994 | 0.3122 | 3.686 | 7.773 |
| | Eₘₐₓ (nM) | 483.5 | 829.6 | 3752 | 13815 |
| Anti-PD-1 antibody clone T5 | EC₅₀ (nM) | nb | nb | 0.2842 | 0.4175 |
| | Eₘₐₓ (nM) | nb | nb | 6241 | 17731 |
| Ctrl anti-4-1BB antibody | EC₅₀ (nM) | 0.5303 | 1.689 | nb | nb |
| | Eₘₐₓ (nM) | 1079 | 18.59 | nb | nb |
| Ctrl anti-PD-1 antibody | EC₅₀ (nM) | nb | nb | 0.2831 | 0.4436 |
| | Eₘₐₓ (nM) | nb | nb | 6722 | 17587 |

nb: not binding

Table 7 summarizes the EC₅₀ and Eₘₐₓ values of “Construct #3” versus control antibodies measured in Jurkat cells expressing either human PD-1 (FIG. 10E) or human 4-1BB (FIG. 10F) at physiological antigen densities.

TABLE 7
EC₅₀ and Eₘₐₓ values in Jurkat cells

| | | Jurkat h4-1BB | Jurkat hPD-1 |
|---|---|---|---|
| Construct #3 | EC₅₀ (nM) | 0.1907 | 12.35 |
| | Eₘₐₓ (nM) | 1232 | 320.8 |
| Anti-PD-1 antibody clone T5 | EC₅₀ (nM) | nb | 0.2776 |
| | Eₘₐₓ (nM) | nb | 280.3 |
| Ctrl anti-4-1BB antibody | EC₅₀ (nM) | 0.1349 | nb |
| | Eₘₐₓ (nM) | 1505 | nb |
| Ctrl anti-PD-1 antibody | EC₅₀ (nM) | nb | 0.3788 |
| | Eₘₐₓ (nM) | nb | 356.6 |

nb: not binding

### Binding Assays on Primary Human T-Cells

Table 8 summarizes the EC₅₀ and Eₘₐₓ values of “Construct #3” versus control antibodies measured in stimulated primary T-cells (FIG. 10G).

TABLE 8
EC₅₀ and Eₘₐₓ values in stimulated primary T-cells

| | | Stimulated T-cells |
|---|---|---|
| Construct #3 | EC₅₀ (nM) | 1.534 |
| | Eₘₐₓ (nM) | 51.52 |
| Anti-PD-1 antibody clone T5 | EC₅₀ (nM) | 0.205 |
| | Eₘₐₓ (nM) | 33.16 |
| Ctrl anti-4-1BB antibody | EC₅₀ (nM) | 0.046 |
| | Eₘₐₓ (nM) | 17.09 |
| Ctrl anti-PD-1 antibody #1 | EC₅₀ (nM) | 0.044 |
| | Eₘₐₓ (nM) | 34.81 |
| Ctrl anti-PD-1 antibody #2 | EC₅₀ (nM) | 0.007 |
| | Eₘₐₓ (nM) | 31.33 |

nb: not binding

### Reporter Assays

Construct #3, used as soluble agent, has similar EC₅₀ and IC₅₀ values as control antibodies in both 4-1BB and PD-1 reporter assays (FIGS. 11A and 11B, respectively; and Table 9).

TABLE 9
EC₅₀ and IC₅₀ values in 4-1BB and PD-1 reporter assays

| | EC₅₀ (nM) | IC₅₀ (nM) |
|---|---|---|
| | 4-1BB reporter | PD-1 reporter |
| Test compound | assay | assay |
| Ctrl anti-4-1BB antibody | 0.4 | nb |
| Ctrl anti-PD-1 antibody | nb | 1.8 |
| Anti-PD-1 antibody clone T5 | nb | 2.8 |
| Construct #3 | 0.1 | 6.7 |

nb: not binding

### ADCC/ADCP/CDC Assays

Construct #3 bears no residual ADCC (FIG. 12A-12B) or ADCP (FIG. 13A-13B) Fc backbone activities, and only weak CDC Fc backbone activity (FIG. 14A-14B).

### T-Cell Activation (TCA)

Construct #3 triggered a superior T-cell activation as soluble agent in a T-cell activation assay (FIGS. 15A-15B for human T-cell activation; FIG. 15C for cyno T-cell activation).

### Mixed Lymphocyte Reaction (MLR) Assay

Construct #3 induced a superior T-cell activation in an MLR assay as soluble agent than the combination of the two monospecific control antibodies but also more than the combination of each respective monospecific arm composing “Construct #3” (FIGS. 16A-16C).

### CD3-PBMC Activation Assay

Construct #3 also triggered multi-cytokine secretion in a CD3-PBMC activation assay (FIG. 17A-17B).

In a dose-response CD3-PBMC activation assay, “Construct #3” appeared to be more potent than the combination of the two monospecific control antibodies (FIG. 18A-18C).

### MIMIC CD8⁺ T-Cell Exhaustion Assay

Construct #3 also reinvigorated exhausted T-cells more than the combination of the two monospecific control antibodies (FIG. 19A). Reinvigorated exhausted CD8⁺ T-cells were again functional, as they could secrete IFN-γ and TNF-α after treatment (FIG. 19B-19C).

### Regulatory T-Cells (T_{reg}) Suppression Assay

Construct #3 induced in vitro T_{reg} suppressive activity, at a ratio of T_{eff}:eT_{reg} cells as low as 2:1, to a higher extent than the control anti-4-1BB antibody (FIG. 20).

### In Vivo Efficacy Study

Outstanding single agent in vivo efficacy was observed in hu 4-1BB+PD-1 dKI mice bearing MC38 tumor model at high (8/9 CR) and at low (6/9 CR) doses (FIG. 21).

### Sequences

The anti-4-1BB clone #5.1 V_{HH} has an amino acid sequence set forth as:

(SEQ ID NO: 3)
EVQLVESGGGVVQPGGSLRLSCAAS [symbol] TMT
WVRQAPGKGLEWVSSI [symbol] IIYADSVKGRFTI
SRDNAKNTLYLQMNSLRPEDTALYYCAR [symbol]
SGQGTLVTVSS

CDR sequences are highlighted: IMGT numbering in bold; Kabat numbering underlined; Chothia numbering italicized.

The anti-4-1BB clone #2.1 V_{HH} has an amino acid sequence set forth as:

(SEQ ID NO: 4)
EVQLVESGGGVVQPGGSLRLSCAAS [symbol] TGG
WYRQAPGKQRELVATI [symbol] TNYAESVKGRFTIS
RDNAKNTVYLQMNSLRPEDTALYYCRL [symbol]
WGQGTLVTVSS

CDR sequences are highlighted: IMGT numbering in bold; Kabat numbering underlined; Chothia numbering italicized.

The light chain (LC) sequence of “Construct #3” is set forth as:

(SEQ ID NO: 9)
EIVLTQSPATLSLSPGERATLSCGASQSVSINF
LAWYQQKPGLAPRLLIYEASSRATGIPDRFSGS
GSGTDFTLTISRLEPEDFAVYYCQQYGSSPYT
FGQGTKLEIKRTVAAPSVFIFPPSDEQLKSGT
ASVVCLLNNFYPREAKVQWKVDNALQSGNSQE
SVTEQDSKDSTYSLSSTLTLSKADYEKHKVYA
CEVTHQGLSSPVTKSFNRGEC

wherein C means inter disulfide bonds; C means intra disulfide bonds; and bolded residue sections are light chain CDRs 1, 2 and 3 of the anti-PD-1 Fab “T5” according to IMGT numbering.

The heavy chain (HC) sequence of “Construct #3” is set forth as:

(SEQ ID NO: 10)
EVQLVESGGGVVQPGGSLRLSCAASGGLFSINT
GGWYRQAPGKQRELVATITHDDRTNYAESVKGR
FTISRDNAKNTVYLQMNSLRPEDTALYYC
RLGSAAIRGYWGQGTLVTVSSGGGGSGGGGSGG
GGSEVQLVESGGGVVQPGGSLRLSCAAS
GFTFSDHTMTWVRQAPGKGLEWVSSISSGGSRI
IYADSVKGRFTISRDNAKNTLYLQMNSLRPEDT
ALYYCARGTRYKLSTSGQGTLVTVSSGGGGSDK
THTCPPCPAPEAAGGPSVFLFPPKPKDTLMISR
TPEVTCVVVDVSHEDPEVKFNWYVDGVEVHNAK
TKPREEQYNSTYRVVSVLTVLHQDWLNGKEYKC
KVSNKALPAPIEKTISKAKGQPREPQVYTLPPS
RDELTKNQVSLTCLVKGFYPSDIAVEWESNGQP
ENNYKTTPPVLDSDGSFFLYSKLTVDKSRWQQG
NVFSCSVMHEALHNHYTQKSLSLSPGGGGGSGG
GGSGGGGSGGGGSGGGGSQLQLQESGPGLVKPS
ETLSLTCTVSGGSISTSSYFWGWIRQPPGKGLE
WIGSIYRSGSTYYNPSLKSRVTISVDTSKNQFS
LKLSSVTAADTAVYYCARGITGDPGDYWGQGTL
VTVSSASTKGPSVFPLAPSSKSTSGGTAALGCL
VKDYFPEPVTVSWNSGALTSGVHTFPAVLQSSG
LYSLSSVVTVPSSSLGTQTYICNVNHKPSNTKV
DKKVEPKSC

wherein C means inter disulfide bonds; C means intra disulfide bonds; the first three bolded residues sections are CDRs 1, 2 and 3 (according to IMGT numbering) of the anti-4-1BB clone #2.1 V_{HH}; the second three bolded residues sections are CDRs 1, 2 and 3 (according to IMGT numbering) of the anti-4-1BB clone #5.1 V_{HH}; and the third three bolded residues sections are heavy chain CDRs 1, 2 and 3 (according to IMGT numbering) of the anti-PD-1 Fab “T5”.

### Example 4

### Optimized Construct #3

The anti-PD-1 Fab “T5” of original “Construct #3” (described in Example 2) was sequence-optimized (“T5_optimized” clone). This T5_optimized clone was inserted in a new “Optimized Construct #3”, in lieu of the original T5 clone.

### Material and Methods

### Expression and Purification

For expression and purification, see Example 3.

### Surface Plasmon Resonance (SPR) Binding Assay

Goal: evaluate the binding affinity of original “Construct #3” versus “Optimized Construct #3” on their targets.

SPR binding assay was carried out on a Biacore 8K instrument using CM5 anti-Fc chips, and either human PD-1 or human 4-1BB as analyte. Original “Construct #3” and “Optimized Construct #3” served as ligand.

The anti-Fc capture antibody was diluted in running buffer 1:20 and coupled to the CM5 chip (Cytiva, Catalog No. 29149603) using standard amine coupling to yield approximately 8000 response units (RU) using the amine coupling kit (Cytiva, Catalog No. BR-100-50).

The ligand was used at a concentration of 0.5 μg/mL, injected for 60 seconds at 10 μg/mL. The analytes, at concentrations of 0.39, 0.78, 1.56, 3.13, 6.25, 12.5, 25, 50 and 100 nM, were injected for 240 seconds at 30 μL/minute followed by a dissociation phase for 1 200 seconds at 30 μL/minute in HBS-EP+ buffer. Regeneration was performed using 3 M MgCl₂ injected for 60 seconds at 30 μL/minute.

Binding kinetics data were evaluated with the Biacore Insight Evaluation Software (Cytiva) using a 1:1 binding model.

### Single Molecule Localization Microscopy (SMLM)

The goal of this experiment was to evaluate and compare the binding of “Construct #3” and of “Optimized Construct #3”, versus constructs devoid of a PD-1 arm, of a 4-1BB arm, or both (i.e., constructs where the relevant arm was replaced by an irrelevant arm).

Jurkat cells expressing 4-1BB and PD-1 were activated during 5 days using flasks precoated with OKT3 (CD3) antibody (Invitrogen 16-0037-85) at 5 μg/mL final for 3 hours at 37° C. Medium was composed of RPMI 1640 (Gibco 21875), 10% fetal bovine serum (Gibco 16629525), 2 mM L-glutamine (Stemcell 07100), 1% non-essential amino acids (Gibco 11140-035), 1 mM sodium pyruvate (Gibco 12539059), 400 μg/mL final of hygromycin B (Invitrogen 10687010) and 600 μg/mL final of geneticin (Gibco 11811-023).

At day 5, 2 assays were run in parallel: a binding assay and a receptor density assay.

Binding assay: 1.10×10⁶ cells were placed in Eppendorf tubes, centrifuged, resuspended with test compounds (at 200 nM, 300 nM, 600 nM and 1200 nM, diluted in RPMI 1640), and incubated for 30 minutes at 37° C. After incubation, cells were fixed with for mol 4% for 20 minutes at 4° C., then labelled after washing with Fcγ fragment specific Alexa Fluor® 647 AffiniPure Fab Fragment Goat anti-human IgG (Jackson 109-607-008) 1/1000 for 1 hour at room temperature. After washing, labelled cells were then spread on MW6 slides (Marienfeld 0117650) precoated with poly-D-lysine (Corning 354210) diluted at 1/400 in PBS for 30 minutes at 37° C. After 10 minutes, the slides were mounted for SMLM acquisition using Smart kit super resolution buffer (Abbelight).

### TCA & MLR Assays

For TCA and MLR assays, see Example 3.

### Results

### Surface Plasmon Resonance (SPR) Binding Assay

As shown in Table 10, the “Optimized Construct #3” demonstrated an improved K_{D} compared to the original “Construct #3” for binding to human PD-1. Binding efficacy to 4-1BB remains unchanged.

TABLE 10
K_{D} values

| Ligand | Analyte | K_{D} (M) | K_{D} (nM) |
|---|---|---|---|
| Original Construct #3 | Human PD-1 | 1.60 × 10⁻⁸ | 16.04 |
| Original Construct #3 | Human 4-1BB | 7.82 × 10⁻⁹ | 7.82 |
| Optimized Construct #3 | Human PD-1 | 4.54 × 10⁻⁹ | 4.54 |
| Optimized Construct #3 | Human 4-1BB | 7.23 × 10⁻⁹ | 7.23 |

### Single Molecule Localization Microscopy (SMLM)

FIGS. 22A-22B show the density of “Construct #3” and “Optimized Construct #3”, respectively (expressed as the number of test compounds bound per μm² of Jurkat cells surface).

Binding of “Construct #3 ΔPD-1” and “Construct #3 Δ4-1BB” (lacking a functional PD-1 arm and 4-1BB arm, respectively) increased in a dose-dependent manner to reach a maximum density at 600 nM. For “Construct #3”, maximum density was reached at 300 nM. Interestingly, there is a significant difference in the density reached at 300 nM with Construct #3 versus the two ΔPD-1 and Δ4-1BB constructs, with “Construct #3” density being higher than the sum of densities of the two ΔPD-1 and Δ4-1BB constructs taken together (FIG. 22A). When comparing binding protein density and receptor (PD-1 and 4-1BB) density on Jurkat cells, we could observe that the 4-1BB arm has a good binding rate on the cell membrane leading to a saturation of 4-1BB antigens, contrary to the PD-1 arm which bound only around 30% of the available PD-1 antigens. We hypothesize that, in “Construct #3”, the 4-1BB arm drives the binding activity.

Binding of “Optimized Construct #3”, “Optimized Construct #3 ΔPD-1” and “Optimized Construct #3 Δ4-1BB” all increased in a dose-dependent manner to reach a maximum density at 600 nM. Contrary to the observation made with “Construct #3” above, we did not observe a significant change in density between these three compounds (FIG. 22B).

When comparing binding protein density and receptor (PD-1 and 4-1BB) density on Jurkat cells, we could observe that both the PD-1 arm and the 4-1BB arm had a good binding rate on the cell membrane leading to a saturation of PD-1 and 4-1BB antigens. We hypothesize that, in “Optimized Construct #3”, the 4-1BB arm was no longer the sole driver of binding activity contrary to “Construct #3”.

### TCA & MLR Assays

The “Optimized Construct #3” outperformed the original “Construct #3” in a T-cell activation assay (FIGS. 23A-23B) and performed substantially similarly in an MLR assay (FIGS. 24A-24B). In both assays, the original “Construct #3” and “Optimized Construct #3” outperformed the monospecific control antibodies.

### Sequences

The optimized anti-PD-1 Fab “T5_optimized” has a light chain variable region (LCVR) amino acid set forth as:

(SEQ ID NO: 7)
EIVLTQSPATLSLSPGERATLSCGAS [symbol] LA
WYQQKPGLAPRLLIY [symbol] SRHTGIPDRFSGSGSGTD
FTLTISRLEPEDFAVYYC [symbol] FGQGTKLEIK

CDR sequences are highlighted: IMGT numbering in bold; Kabat numbering underlined; Chothia numbering italicized.

The optimized anti-PD-1 Fab “T5_optimized” has a heavy chain variable region (HCVR) amino acid set forth as:

(SEQ ID NO: 8)
QLQLQESGPGLVKPSETLSLTCTVS [symbol] FWG
WIRQPPGKGLEWIGSI [symbol] TYYNPSLKSRVTISVD
TSKNQFSLKLSSVTAADTAVYYCAR [symbol] WGQ
GTLVTVSS

CDR sequences are highlighted: IMGT numbering in bold; Kabat numbering underlined; Chothia numbering italicized.

The light chain (LC) sequence of “Optimized Construct #3” is set forth as:

(SEQ ID NO: 11)
EIVLTQSPATLSLSPGERATLSCGASQSVPINFLAWYQ
QKPGLAPRLLIYEASSRHTGIPDRFSGSGSGTDFTLTI
SRLEPEDFAVYYCGQYGSSPYTFGQGTKLEIKRTVAAP
SVFIFPPSDEQLKSGTASVVCLLNNFYPREAKVQWKVD
NALQSGNSQESVTEQDSKDSTYSLSSTLTLSKADYEKH
KVYACEVTHQGLSSPVTKSFNRGEC

wherein C means inter disulfide bonds; C means intra disulfide bonds; and bolded residue sections are light chain CDRs 1, 2 and 3 (according to IMGT numbering) of the anti-PD-1 Fab “T5_optimized”.

The heavy chain (HC) sequence of “Optimized Construct #3” is set forth as:

(SEQ ID NO: 12)
EVQLVESGGGVVQPGGSLRLSCAASGGLFSINTGGWY
RQAPGKQRELVATITHDDRTNYAESVKGRFTISRDNA
KNTVYLQMNSLRPEDTALYYCRLGSAAIRGYWGQGTL
VTVSSGGGGSGGGGSGGGGSEVQLVESGGGVVQPGGS
LRLSCAASGFTFSDHTMTWVRQAPGKGLEWVSS
ISSGGSRIIYADSVKGRFTISRDNAKNTLYLQMNSLR
PEDTALYYCARGTRYKLSTSGQGTLVTVSSGGGGSDK
THTCPPCPAPEAAGGPSVFLFPPKPKDTLMISRTPEV
TCVVVDVSHEDPEVKFNWYVDGVEVHNAKTKPREEQY
NSTYRVVSVLTVLHQDWLNGKEYKCKVSNKALPAPIE
KTISKAKGQPREPQVYTLPPSRDELTKNQVSLTCLVK
GFYPSDIAVEWESNGQPENNYKTTPPVLDSDGSFFLY
SKLTVDKSRWQQGNVFSCSVMHEALHNHYTQKSLSLS
PGGGGGSGGGGSGGGGSGGGGSGGGGSQLQLQESGPG
LVKPSETLSLTCTVSGGSISSSSYFWGWIRQPPGKGL
EWIGSIYRSGSTYYNPSLKSRVTISVDTSKNQFSLKL
SSVTAADTAVYYCARGITGDPGDYWGQGTLVTVSSAS
TKGPSVFPLAPSSKSTSGGTAALGCLVKDYFPEPVTV
SWNSGALTSGVHTFPAVLQSSGLYSLSSVVTVPSSSL
GTQTYICNVNHKPSNTKVDKKVEPKSC

wherein C means inter disulfide bonds; C means intra disulfide bonds; the first three bolded residues sections are CDRs 1, 2 and 3 (according to IMGT numbering) of the anti-4-1BB clone #2.1 V_{HH}; the second three bolded residues sections are CDRs 1, 2 and 3 (according to IMGT numbering) of the anti-4-1BB clone #5.1 V_{HH}; and the third three bolded residues sections are heavy chain CDRs 1, 2 and 3 (according to IMGT numbering) of the anti-PD-1 Fab “T5_optimized”.

### Example 5

### Conditionally-Active Anti-4-1BB/Anti-PD-1 Bispecific Binding Proteins

We generated conditionally-active anti-4-1BB/anti-PD-1 bispecific binding proteins from “Optimized Construct #3” described in Example 3.

Briefly, the conditionally-active bispecific binding proteins comprise, from N- to C-terminal:
a masked anti-4-1BB V_{HH} (clone #2.1);
an anti-4-1BB V_{HH} (clone #5.1);
an IgG1-LALA Fc region; and
an anti-PD-1 Fab (clone “T5_optimized”).

The masked anti-4-1BB V_{HH} clone #2.1 itself comprises, from N-terminal to C-terminal, a masking moiety (MM), a cleavable linker and the anti-4-1BB V_{HH} clone #2.1.

Cleavable linkers typically comprise a short amino acid sequence which is the target for a protease. In the case of cancer treatment, it is preferable that the protease be a tumor-specific protease, i.e., a protease which is, if not exclusively, at least predominantly found in the tumor microenvironment in vivo. Hence, in absence of protease, the masking moiety remains fused to the bispecific binding protein, thereby reducing, inhibiting or abrogating the binding of the bispecific binding protein to its target (here, of the masked anti-4-1BB arm to its target, 4-1BB). However, when the conditionally-active bispecific binding protein colocalizes with a protease capable of cleaving the cleavable linker, e.g., in a tumor microenvironment, the masking moiety is released from the bispecific binding protein and binding of the latter to the target antigen (e.g., 4-1BB) is restored.

Two alternative masking moieties (MM1 and MM2) were identified following Adagene's protocol using a synthetic library as described in WO 2019/149282 A1; MM1 or MM2 were linked to the N-terminus of the anti-4-1BB V_{HH} clone #2.1 through one of two cleavable linkers (cleavable linker #1 or cleavable linker #2). Combining one masking moiety with one cleavable linker led to four different conditionally-active (masked) bispecific binding proteins:
MC1, comprising MM1 and cleavable linker #1;
MC2, comprising MM2 and cleavable linker #1;
MC3, comprising MM1 and cleavable linker #2; and
MC4, comprising MM2 and cleavable linker #2.

### Material and Methods

### Expression and Purification

For expression and purification, see Example 3.

### MMP9-Mediated Cleavage

For experiments requiring cleavage of a masking moiety by MMP9 protease (compounds identified as “MMP9-activated” hereafter).

In a first step, recombinant human MMP9 protein (R&D Systems, Ref. 911-MP-010) was activated using p-aminophenylmercuric acetate (APMA; Calbiochem, Ref. 164610-700MG), added to a final concentration of 1 mM into MMP9 solution at 100 μg/mL and incubated at 37° C. for 24 hours. Activated MMP9 was then aliquoted and stored at −80° C. until further use.

Then, test (masked) compounds were diluted to 1 mg/mL and incubated for 24 hours at 37° C. under gentle shake (300 rpm) with activated MMP9 in a final concentration of 5 nM.

If desired, to remove excess masking peptide, the samples were purified using a HiLoad® 26/600 Superdex® 200 (GE Healthcare, Ref. 28-9893-36).

### Surface Plasmon Resonance (SPR) Binding Assay

Goal: evaluate the binding affinity of “Optimized Construct #3” versus conditionally-active anti-4-1BB/anti-PD-1 bispecific binding proteins MC1-MC4 and demasked versions thereof on their targets.

SPR binding assay was carried out on a Biacore 8K instrument using CM5 anti-Fc chips, and either human PD-1 or human 4-1BB as analyte. The test compounds served as ligand.

Anti-Fc capture antibody was diluted in running buffer 1:20 and coupled to the CM5 chip (Cytiva, Catalog No. 29149603) using standard amine coupling to yield approximately 8000 response units (RU) using the amine coupling kit (Cytiva, Catalog No. BR-100-50).

The ligand was used at a concentration of 0.5 μg/mL, injected for 60 seconds at 10 μg/mL. The analytes, at concentrations of 0.39, 0.78, 1.56, 3.13, 6.25, 12.5, 25, 50 and 100 nM, were injected for 240 seconds at 30 μL/minute followed by a dissociation phase for 1 200 seconds at 30 μL/minute in HBS-EP+ buffer. Regeneration was performed using 3 M MgCl₂ injected for 60 seconds at 30 μL/minute.

Binding kinetics data were evaluated with the Biacore Insight Evaluation Software (Cytiva) using a 1:1 binding model.

### Binding Assay on Stable Cell Lines

### Human PD-1 and 4-1BB

Goal: evaluate the affinity of “Optimized Construct #3” versus conditionally-active anti-4-1BB/anti-PD-1 bispecific binding proteins MC1-MC4 and demasked versions thereof on their targets. Calculate the binding EC₅₀ and the Eₘₐₓ values of the test compounds.

The binding assay was performed in 96-well plates on ice using PD-1 NFAT-luc2 Jurkat cells (Promega #J1252), or 4-1BB NF-κB-luc2P Jurkat cells (Promega #J2332), or in-house transfected 300.19 (pre-B) cells expressing either human PD-1 (hPD-1) or human 4-1BB (h4-1BB).

A cell suspension was plated at a density of 50×10³ cells per well in 96-well U bottom plates. Serially diluted concentrations of test compounds were added to the cells for 1 hour. After washing, a fluorescently labeled secondary antibody targeting the Fc portion was added to each well for 30 minutes. The MFI signal was then measured using a flow cytometer.

Data from the flow cytometer was analyzed using FlowJo (V10.8.1), then the binding curves, the Eₘₐₓ, and EC₅₀ values were plotted using GraphPad Prism (V9.1.2).

### Cynomolgus Monkey PD-1 and 4-1BB

Goal: evaluate the affinity of “Optimized Construct #3” versus conditionally-active anti-4-1BB/anti-PD-1 bispecific binding proteins MC1-MC4 and demasked versions thereof on their targets. Calculate the binding EC₅₀ and the Eₘₐₓ values of the test compounds.

The binding assay was performed in 96-well plates on ice using in-house transfected 300.19 (pre-B) cells expressing either cyno PD-1 (cyPD-1) or cyno 4-1BB (cy4-1BB).

A cell suspension was plated at a density of 50×10³ cells per well in 96-well U bottom plates. Serially diluted concentrations of test compounds were added to the cells for 1 hour. After washing, a fluorescently labeled secondary antibody targeting the Fc portion was added to each well for 30 minutes. The MFI signal was then measured using a flow cytometer.

Data from the flow cytometer was analyzed using FlowJo (V10.8.1), then the binding curves, the Eₘₐₓ, and EC₅₀ values were plotted using GraphPad Prism (V9.1.2).

### Reporter Assays

### 4-1BB

Goal: Assess the 4-1BB arm activity of “Optimized Construct #3” versus conditionally-active anti-4-1BB/anti-PD-1 bispecific binding proteins MC1-MC4 and demasked versions thereof. Calculate the EC50 and the Eₘₐₓ for each of the test compounds.

Readout using the 4-1BB reporter Jurkat NF-κB-luc2P Promega cell line (GloResponse NF-κB-luc2P Jurkat cells, Promega #J2332) cultured in RPMI 1640, 10% SVF, 1% glutamine, 1% NEAA, 1 mM sodium pyruvate, 800 μg/mL G418, 500 μg/mL hygromycin following the manufacturer's instructions.

Cells were plated at a density of 50×10³ cells per well in 96-well white flat bottom plates. Serially diluted concentrations of test compounds were added. After an incubation of 6 hours at 37° C., Bio-Glo Reagent was added to each well. Luminescence was measured using an Infinit Pro M1000 or a SPARK TECAN reader.

The binding curves, Eₘₐₓ, and EC₅₀ values were plotted using GraphPad Prism (V9.1.2).

### PD-1

Goal: Assess the PD-1 arm activity of “Optimized Construct #3” versus conditionally-active anti-4-1BB/anti-PD-1 bispecific binding proteins MC1-MC4 and demasked versions thereof. Calculate the IC₅₀ and the Eₘₐₓ for each of the test compounds.

Readout using the PD-1 reporter Jurkat NFAT-luc2 Promega cell line (GloResponse PD-1 NFAT-luc2 Jurkat cells, Promega #J1252) cultured in RPMI 1640, 10% SVF, 1% glutamine, 1% NEAA, 1 mM PyNa, 500 μg/mL G418, 200 μg/mL hygromycin in presence of the PD-L1 aAPC/CHO-K1 accessory cells (Promega #J1252) cultured in HAM/F12, 10% SVF, 200 μg/mL hygromycin, 250 μg/mL G418, following the manufacturer's instructions.

PD-L1 aAPC/CHO-K1 were plated at a density of 40×10³ cells per well in 96-wells white flat bottom culture plates and were incubated overnight at 37° C. in Ham/F12 media supplemented with 1% FCS. The following day, media was removed, a PD-1 NFAT-luc2 Jurkat cells suspension at a density of 50×10³ cells/well was added in the wells, and serially diluted concentrations of test compounds were added. After an incubation of 6 hours at 37° C., Bio-Glo Reagent was added to each well. Luminescence was measured using an Infinit Pro M1000 or a SPARK TECAN reader.

The binding curves, Imax, and IC₅₀ values were plotted using GraphPad Prism (V9.1.2).

### T-Cell Activation (TCA) Assay

### Human TCA Assay

The in vitro biological activity of “Optimized Construct #3” (without masking moiety), of each of MC1-MC4 (masked), and of protease-activated compounds (de-masked) was measured using CellTiter-Glo (CTG) method.

Briefly, the peripheral blood mononuclear cells (PBMCs) were first isolated from fresh human blood and then the total T-cells were purified with StemCell kit. These cells (1×10⁵ cells per well) were incubated in 96-well tissue culture plates pre-coated with suboptimal concentration (5 μg/mL) of anti-human CD3, in the presence or absence of serial dilutions of test articles. The level of T-cell activation was then measured by T-cell proliferation with CTG method.

### Murine TCA Assay

Goal: Assess the activity of “Optimized Construct #3” versus conditionally-active anti-4-1BB/anti-PD-1 bispecific binding proteins MC1-MC4 and demasked versions thereof, versus isotype control. Observe the cytokine release from activated murine T-cells.

C57BL/6-Tnfrsf9^{tm1(INFRSF9)}/Bcgen mice (humanized h4-1BB KI) from Biocytogen (Ref. 110004) were crossed with hPD-1 KI mice from CIPHE (Center for Immunophenomics, Marseille, France) to generate a novel h4-1BB/hPD-1 double KI (dKI) mouse. This dKI mouse model was validated genotypically and phenotypically. Bridging of the new mouse model was also validated.

Murine T-cells were isolated from splenocytes of the dKI mouse via a negative selection. T-cells were plated at a density of 200×10³ cells/well in 96-flat bottom well plates previously coated with 1 μg/mL of anti-mouse CD3, and cultured in complete media comprising RPMI 1640 (Gibco 31870-025), 2 mM L-glutamine (Gibco 25030-081), 10% FCS (Eurobio CVFSF00-01), 1× non-essential amino acids (Gibco 11140-035), 1 mM sodium pyruvate (Gibco 11360-070), 0.05 mM 2-mercaptoethanol (Gibco 31350-010), supplemented with 1% penicillin/streptomycin.

Serially diluted concentrations of test compounds were added to the culture. After 2 days of incubation, cytokine secretions were evaluated in the harvested supernatants using a mouse Th1/Th2 cytokine cytometric bead array (CBA) kit on a flow cytometer according to manufacturer's instructions (BD Bioscience 551287).

Cytokine secretions were analyzed with FCAP array (v3.0.19.2091), then plotted using GraphPad Prism (v9.5.0).

### Mixed Lymphocyte Reaction (MLR) Assay

Goal: Assess the activity of “Optimized Construct #3” versus conditionally-active anti-4-1BB/anti-PD-1 bispecific binding proteins MC1-MC4 and demasked versions thereof in an MLR assay. Observe the cytokine release from the co-culture.

PBMC were isolated from fresh buffy coats using a Ficoll gradient. Monocytes were isolated via a positive selection. Monocytes were cultured for 6 days in presence of 50 ng/ml GM-CSF and 10 ng/ml IL-4 in RPMI 1640, 10% FCS, 2 mM glutamine, 1% penicillin/streptomycin to differentiate them into Mo-DCs. In parallel, PBMC were isolated from fresh buffy coats from another donor using a Ficoll gradient. T-cells were isolated via a negative selection.

A mixture of heterologous T-cells and Mo-DCs at a ratio 10:1 T-cells:Mo-DCs was plated in 96-well U bottom plates and cultured in X-Vivo 15 media (Lonza #BE02-061Q) supplemented with 1% penicillin/streptomycin.

Serially diluted concentrations of test compounds were added to the co-culture. After 4 or 6 days of incubation, the cytokines secretion was evaluated in the harvested supernatants either with a CBA human Th1/Th2 cytokines kit on a flow cytometer according to the manufacturer's instructions (BD Bioscience #550749), or with a Homogeneous Time Resolved Fluorescence (HTRF) human IFN-γ/TNF-α cytokines kit (Cisbio #62HIFNGPEH and #62HTNFAPEH) on a PHERAstar FSX multimode reader (BMG Labtech) according to the manufacturer's instructions.

The cytokines secretion was analyzed with FCAP array (V3.0.19.2091) for CBA samples, then plotted using GraphPad Prism (V9.1.2). HTRF cytokines secretion was directly plotted using GraphPad Prism (V9.1.2).

### CD3-PBMC Activation Assay

Goal: Assess the activity of “Optimized Construct #3” versus conditionally-active anti-4-1BB/anti-PD-1 bispecific binding proteins MC1-MC4 and demasked versions thereof in a CD3-PBMC activation assay. Observe the cytokine release from PBMC.

PBMC were isolated from fresh buffy coats and plated at a density of 150×10³ cells/well and cultured in presence of 0.04 μg/mL soluble anti-CD3 and serially diluted concentrations of test compounds. After 4 or 6 days of incubation, the cytokines secretion was evaluated in the harvested supernatants either with a CBA human Th1/Th2 cytokines kit on a flow cytometer according to the manufacturer's instructions (BD Bioscience #550749), or with a Homogeneous Time Resolved Fluorescence (HTRF) human IFN-γ/TNF-α cytokines kit (Cisbio #62HIFNGPEH and #62HTNFAPEH) on a PHERAstar FSX multimode reader (BMG Labtech) according to the manufacturer's instructions.

The cytokines secretion was analyzed with FCAP array (V3.0.19.2091) for CBA samples, then plotted using GraphPad Prism (V9.1.2). HTRF cytokines secretion was directly plotted using GraphPad Prism (V9.1.2).

### Plasma Stability

The stability of each of MC1-MC4 (masked) was assessed in mouse, cynomolgus and human plasma over 7 days.

Briefly, each of MC1-MC4 was diluted in mouse, cynomolgus or human EDTA plasma at a final concentration of 100 μg/mL. Samples were incubated for 7 days (i.e., 168 hours) at 37° C., then analyzed by ELISA. Total fraction (cleaved and uncleaved compounds) was detected using an immobilized anti-human IgG Fc antibody and anti-human IgG Fab-HRP conjugate for detection; the cleaved fraction was detected using an immobilized truncated 4-1BB (only capable of binding to the anti-4-1BB clone #2.1 V_{HH}) and anti-human IgG Fab-HRP conjugate for detection.

### In Vivo Efficacy Study

C57BL/6-Tnfrsf9^{tm1(TNFRSF9)}/Bcgen mice (humanized h4-1BB KI) from Biocytogen (Ref. 110004) were crossed with hPD-1 KI mice from CIPHE (Center for Immunophenomics, Marseille, France) to generate a novel h4-1BB/hPD-1 double KI (dKI) mouse model. This dKI mouse model was validated genotypically and phenotypically. These dKI mice were subcutaneously implanted with MC38 tumor cells and randomized when tumors were established (≈100 mm³). Test compounds were administered directly post randomization by i.p. route in a Q3D regimen.

### Results

### Surface Plasmon Resonance (SPR) Binding Assay

As seen in FIG. 25, the four conditionally-active anti-4-1BB/anti-PD-1 bispecific binding proteins MC1-MC4 showed 4-1BB binding only after MMP9-mediated cleavage of their masking moiety (Table 11).

PD-1 binding on the other hand was not affected by the masking moiety (Table 12).

All positive and negative controls showed expected target binding behavior.

TABLE 11
SPR binding on human and cyno 4-1BB

| | K_{D} (nM) | K_{D} (nM) | Ratio K_{D} |
|---|---|---|---|
| Sample | h4-1BB | cy4-1BB | cyno/human |
| Control anti-4-1BB antibody | 6.06 | 296* | 48.8* |
| Construct #3 | 11.84 | 25.1 | 2.1 |
| Optimized Construct #3 | 10.87 | 21.1 | 1.9 |
| MC1 | nb | nb | n/a |
| MC1 MMP9-activated | 6.65 | 29.9 | 4.5 |
| MC2 | nb | nb | n/a |
| MC2 MMP9-activated | 7.57 | 28.7 | 3.8 |
| MC3 | nb | nb | n/a |
| MC3 MMP9-activated | 7.79 | 30.1 | 3.9 |
| MC4 | nb | nb | n/a |
| MC4 MMP9-activated | 6.51 | 21.7 | 3.3 |

*Control anti-4-1BB antibody is not cyno cross-reactive.

h4-1BB: human 4-1BB; cy4-1BB: cynomolgus monkey 4-1BB; nb: not binding; n/a: not applicable.

TABLE 12
SPR binding on human and cyno PD-1

| | K_{D} (nM) | K_{D} (nM) | Ratio K_{D} |
|---|---|---|---|
| Sample | hPD-1 | cyPD-1 | cyno/human |
| Control anti-PD-1 antibody | 7.36 | 3.12 | 0.4 |
| Construct #3 | 7.05 | 18.2 | 2.6 |
| Optimized Construct #3 | 7.29 | 14.9 | 2.0 |
| MC1 | 6.42 | 14.9 | 2.3 |
| MC1 MMP9-activated | 6.89 | 10.7 | 1.6 |
| MC2 | 7.73 | 17.3 | 2.2 |
| MC2 MMP9-activated | 7.40 | 15.0 | 2.0 |
| MC3 | 6.73 | 13.4 | 2.0 |
| MC3 MMP9-activated | 7.07 | 13.0 | 1.8 |
| MC4 | 6.82 | 18.3 | 2.7 |
| MC4 MMP9-activated | 26.5 | 57.4 | 2.2 |

hPD-1: human PD-1;
cyPD-1: cynomolgus monkey PD-1.

### Binding Assay on Stable Cell Lines

### Pre-B Cells

On human 300.19 pre-B-cells, the conditionally-active anti-4-1BB/anti-PD-1 bispecific binding proteins with cleavable linker #1 (MC1 and MC2) had an EC₅₀ towards 4-1BB higher than those with cleavable linker #2 (MC3 and MC4).

Conditionally-active anti-4-1BB/anti-PD-1 bispecific binding proteins bear a 1.5- to 2-log masking efficacy in human 4-1BB+ cells (FIG. 26A) and a 0.5- to 1-log masking efficacy in cyno 4-1BB+ cells (FIG. 26B). After MMP9-mediated cleavage of their masking moiety, all four bispecific binding proteins had an equivalent EC₅₀ to “Optimized Construct #3” (unmasked) (Table 13).

PD-1 binding on the other hand was not affected by the masking moiety (FIGS. 26C-26D; Table 14).

All positive and negative controls showed expected target binding behavior.

TABLE 13
binding on h4-1BB⁺ or cy4-1BB⁺ 300.19 pre-B-cells

| | h4-1BB | | cy4-1BB | | | | h/cy4-1BB |
|---|---|---|---|---|---|---|---|
| | Top | h4-1BB | Top | cy4-1BB | Ratio | h/cy4-1BB | binding |
| | value | EC₅₀ | value | EC₅₀ | EC₅₀ | masking | recovery |
| | (MFI) | (nM) | (MFI) | (nM) | cy/h | efficacy** | (%)*** |
| Control | 5807 | 0.25 | 125 | unstable | n/a | n/a | n/a |
| anti-4-1BB | | | | | | | |
| antibody* | | | | | | | |
| Optimized | 2841 | 0.27 | 3566 | 0.91 | 0.30 | 1/1 | 100/100 |
| Construct #3 | | | | | | | |
| MC1 | 5139 | 83.79 | 4183 | 6.24 | 0.07 | 310/7 | n/a |
| MC1 | 3043 | 0.18 | 3741 | 0.70 | 3.89 | n/a | 100/100 |
| MMP9-activated | | | | | | | |
| MC2 | 2537 | 6.81 | 4366 | 16.64 | 2.44 | 25/18 | n/a |
| MC2 | 2824 | 0.14 | 3636 | 0.94 | 6.71 | n/a | 99/100 |
| MMP9-activated | | | | | | | |
| MC3 | 5744 | 25.57 | 4631 | 5.90 | 0.23 | 95/6 | n/a |
| MC3 | 3050 | 0.18 | 3581 | 0.61 | 3.39 | n/a | 100/100 |
| MMP9-activated | | | | | | | |
| MC4 | 3775 | 9.70 | 4254 | 9.81 | 1.01 | 36/11 | n/a |
| MC4 | 3245 | 0.15 | 3810 | 0.94 | 6.27 | n/a | 100/100 |
| MMP9-activated | | | | | | | |

*Control anti-4-1BB antibody is not cyno cross-reactive.
**Masking efficacy corresponds to the ratio of masked (i.e., MC1-MC4) EC₅₀/naked (i.e., “Optimized Construct #3”) EC₅₀ values.
***Binding recovery corresponds to the % difference between naked (i.e., “Optimized Construct #3”) and demasked (i.e., MC1-MC4 MMP9-activated) top values.
h4-1BB: human 4-1BB; cy4-1BB: cynomolgus monkey 4-1BB; n/a: not applicable.

TABLE 14
binding on hPD-1⁺ or cyPD-1⁺ 300.19 pre-B-cells

| | hPD-1 | hPD-1 | cyPD-1 | cyPD-1 | Ratio |
|---|---|---|---|---|---|
| | Top value | EC₅₀ | Top value | EC₅₀ | EC₅₀ |
| | (MFI) | (nM) | (MFI) | (nM) | cy/h |
| Control anti-PD-1 | 22620 | 0.15 | 38204 | 0.71 | 4.73 |
| antibody | | | | | |
| Optimized | 19725 | 1.40 | 17494 | 1.45 | 1.04 |
| Construct #3 | | | | | |
| MC1 | 19295 | 1.65 | 19869 | 1.67 | 0.99 |
| MC1 | 19833 | 1.74 | 17368 | 1.18 | 0.68 |
| MMP9-activated | | | | | |
| MC2 | 20165 | 2.97 | 20915 | 1.50 | 0.51 |
| MC2 | 18618 | 1.62 | 17342 | 1.98 | 1.22 |
| MMP9-activated | | | | | |
| MC3 | 19188 | 1.76 | 18962 | 1.40 | 0.80 |
| MC3 | 19481 | 1.36 | 17882 | 1.55 | 1.14 |
| MMP9-activated | | | | | |
| MC4 | 19166 | 2.68 | 23131 | 4.80 | 1.79 |
| MC4 | 18733 | 1.56 | 18174 | 1.87 | 1.20 |
| MMP9-activated | | | | | |

hPD-1: human PD-1;
cyPD-1: cynomolgus monkey PD-1.

### Jurkat T-Cells

As shown in Table 15 and in FIG. 27, the protease-activated (i.e., de-masked) forms (MMP9-activated and uPA-activated) exhibit similar binding activity to Jurkat/NF-κB-4-1BB cells compared to the parental “Optimized Construct #3” based on EC₅₀ values. Binding is reduced by at least 166-fold for all masked compounds MC1-MC4 relative to “Optimized Construct #3”, and up to 553-fold for MC1.

TABLE 15
EC₅₀ and masking efficacy values

| | | Relative |
|---|---|---|
| Test articles | EC₅₀ (nM) | masking efficacy |
| Optimized Construct #3 (unmasked) | 0.48 | 1 |
| MC1 | 266.2 | 553 |
| MC2 | 123.0 | 256 |
| MC3 | 79.6 | 166 |
| MC4 | 79.7 | 166 |
| MMP9-activated (MC1/MC2) | 0.52 | 1.1 |
| uPA-activated (MC3/MC4) | 0.67 | 1.4 |
| VHH null control | n.a. | n.a. |

In a further experiment on Jurkat T-cells, the conditionally-active anti-4-1BB/anti-PD-1 bispecific binding proteins with cleavable linker #1 (MC1 and MC2) had an EC₅₀ towards 4-1BB higher than those with cleavable linker #2 (MC3 and MC4).

Conditionally-active anti-4-1BB/anti-PD-1 bispecific binding proteins bear a 1.5- to 2-log masking efficacy in human 4-1BB+ cells (FIG. 28A). After MMP9-mediated cleavage of their masking moiety, all four bispecific binding proteins had an equivalent EC₅₀ to “Optimized Construct #3” (unmasked) (Table 16).

PD-1 binding on the other hand was not affected by the masking moiety (FIG. 28B; Table 17).

All positive and negative controls showed expected target binding behavior.

TABLE 16
binding on 4-1BB⁺ Jurkat T-cells

| | Top | EC50 | | Binding |
|---|---|---|---|---|
| | value | value | Masking | recovery |
| Test articles | (MFI) | (nM) | efficacy* | (%)** |
| Control anti-4-1BB antibody | 645 | 0.22 | n/a | n/a |
| Optimized Construct #3 | 525 | 0.48 | 1 | 100% |
| MC1 | 661 | 111.6 | 233 | n/a |
| MC1 MMP9-activated | 587 | 0.54 | n/a | 100% |
| MC2 | 575 | 114.0 | 238 | n/a |
| MC2 MMP9-activated | 594 | 0.61 | n/a | 100% |
| MC3 | 604 | 24.41 | 51 | n/a |
| MC3 MMP9-activated | 590 | 0.55 | n/a | 100% |
| MC4 | 649 | 36.09 | 75 | n/a |
| MC4 MMP9-activated | 592 | 0.50 | n/a | 100% |

*Masking efficacy corresponds to the ratio of masked (i.e., MC1-MC4) EC₅₀/naked (i.e., “Optimized Construct #3”) EC₅₀ values.
**Binding recovery corresponds to the % difference between naked (i.e., “Optimized Construct #3”) and demasked (i.e., MC1-MC4 MMP9-activated) top values.

TABLE 17
binding on PD-1⁺ Jurkat T-cells

| Test articles | Top value (MFI) | EC₅₀ value (nM) |
|---|---|---|
| Control anti-PD-1 antibody | 488 | 0.06 |
| Optimized Construct #3 | 457 | 0.50 |
| MC1 | 483 | 0.70 |
| MC1 MMP9-activated | 493 | 0.85 |
| MC2 | 466 | 0.78 |
| MC2 MMP9-activated | 457 | 0.46 |
| MC3 | 502 | 0.89 |
| MC3 MMP9-activated | 472 | 0.55 |
| MC4 | 476 | 0.72 |
| MC4 MMP9-activated | 451 | 0.40 |

### Reporter Assay

As shown in Table 18 and in FIG. 29, the protease-activated (i.e., cleaved) forms (MMP9-activated and uPA-activated) exhibit similar reporter gene activities compared to the parental “Optimized Construct #3” based on EC₅₀ values. EC₅₀ values are shifted relative to “Optimized Construct #3” by at least 67-fold with masked compounds MC1-MC4, and up to 121-fold for MC1.

TABLE 18
EC₅₀ and masking efficacy values

| | | Relative |
|---|---|---|
| Test articles | EC₅₀ (nM) | masking efficacy |
| Optimized Construct #3 (unmasked) | 0.95 | 1 |
| MC1 | 114.3 | 121 |
| MC2 | 83.3 | 88 |
| MC3 | 89.7 | 95 |
| MC4 | 63.0 | 67 |
| MMP9-activated (MC1/MC2) | 1.1 | 1.1 |
| uPA-activated (MC3/MC4) | 1.1 | 1.2 |
| V_{HH} null control | 139 | 147 |

In a further experiment, “Optimized Construct #3” showed a clear 4-1BB activity superior to that of the control anti-4-1BB antibody; and a clear PD-1 activity superior to that of the control anti-PD-1 antibody.

The conditionally-active anti-4-1BB/anti-PD-1 bispecific binding proteins with cleavable linker #1 (MC1 and MC2) had an EC₅₀ higher in the 4-1BB reporter assay than those with cleavable linker #2 (MC3 and MC4), although in an equivalent nM range.

Conditionally-active anti-4-1BB/anti-PD-1 bispecific binding proteins bear a 1.5- to 2-log masking efficacy (FIG. 30A). After MMP9-mediated cleavage of their masking moiety, all four bispecific binding proteins had an equivalent EC₅₀ to “Optimized Construct #3” (unmasked) (Table 19).

On the other hand, the masking moieties and cleavable linkers did not affect the PD-1 arm functionality (FIG. 30B; Table 20).

All positive and negative controls showed expected target binding behavior.

TABLE 19
4-1BB reporter assay

| Test articles | EC₅₀ value (nM) | Masking efficacy* |
|---|---|---|
| Control anti-4-1BB antibody | 0.33 | n/a |
| Optimized Construct #3 | 0.18 | 1 |
| MC1 | 18.11 | 101 |
| MC1 MMP9-activated | 0.25 | n/a |
| MC2 | 15.13 | 84 |
| MC2 MMP9-activated | 0.22 | n/a |
| MC3 | 7.28 | 40 |
| MC3 MMP9-activated | 0.24 | n/a |
| MC4 | 5.79 | 32 |
| MC4 MMP9-activated | 0.23 | n/a |

*Masking efficacy corresponds to the ratio of masked (i.e., MC1-MC4) EC₅₀/naked (i.e., “Optimized Construct #3”) EC₅₀ values.

TABLE 20
PD-1 reporter assay

| Test articles | Top value (S/N) | EC₅₀ value (nM) |
|---|---|---|
| Control anti-PD-1 antibody | 3.44 | 0.38 |
| Optimized Construct #3 | 3.20 | 1.39 |
| MC1 | 3.15 | 2.43 |
| MC1 MMP9-activated | 2.99 | 0.88 |
| MC2 | 2.99 | 2.54 |
| MC2 MMP9-activated | 2.72 | 0.84 |
| MC3 | 3.25 | 2.62 |
| MC3 MMP9-activated | 3.01 | 1.16 |
| MC4 | 3.13 | 2.81 |
| MC4 MMP9-activated | 2.95 | 1.01 |

### Human T-Cell Proliferation and Activation Assay

As shown in Table 21 and in FIG. 31, at least a 105-fold shift in EC₅₀ was observed for each masked compounds MC1-MC4 relative to the parental “Optimized Construct #3” in a T-cell proliferation assay, and up to 210-fold for MC1.

TABLE 21
EC₅₀ and masking efficacy values

| | | Masking | |
|---|---|---|---|
| Test article | EC₅₀ (nM) | efficacy | Max RLU |
| Optimized Construct #3 (unmasked) | 0.15 | 1 | 680000 |
| MC1 | 35.5 | 210 | 710000 |
| MC2 | 32.5 | 193 | 750000 |
| MC3 | 17.7 | 105 | 680000 |
| MC4 | 19.8 | 117 | 700000 |
| MMP9-activated (MC1/MC2) | 0.17 | 1.1 | n.a. |
| uPA-activated (MC3/MC4) | 0.17 | 1.1 | n.a. |
| V_{HH} null control | n.a. | n.a. | n.a. |

Comparable results were obtained in a T-cell activation assay (IFN-γ release), as shown in Table 22.

TABLE 22
EC₅₀ and masking efficacy values

| Test article | EC₅₀ (nM) | Masking efficacy |
|---|---|---|
| Optimized Construct #3 (unmasked) | 0.22 | 1 |
| MC1 | 140 | 650 |
| MC2 | nd | >1500 |
| MC3 | nd | >1500 |
| MC4 | 96 | 430 |
| MMP9-activated (MC1/MC2) | 0.47 | 2.1 |
| uPA-activated (MC3/MC4) | 0.32 | 1.5 |
| V_{HH} null control | n.a. | n.a. |

### Murine T-Cell Activation (TCA) Assay

All test compounds induced the secretion of IL-4 with a dose-dependent effect (FIG. 32A). Similar observations were shown for IL-2 (FIG. 32B), IFN-γ (FIG. 32C), and TNF-α (FIG. 32D).

The conditionally-active anti-4-1BB/anti-PD-1 bispecific binding proteins with cleavable linker #1 (MC1 and MC2) had an EC₅₀ higher than those with cleavable linker #2 (MC3 and MC4), although in an equivalent nM range (Table 23).

After MMP9-mediated cleavage of their masking moiety, all four bispecific binding proteins were more active than their masked counterparts MC1-MC4, with an EC₅₀ similar to “Optimized Construct #3” (unmasked). Overall, we observed a donor- and cytokine-dependent 1.5- to 2-log masking shift.

Masking shift was similar between human and mouse T-cells, granting species translatability.

TABLE 23
murine T-cell activation assay, IL-4 readout

| Compound | EC₅₀ (nM) | Masking efficacy |
|---|---|---|
| Optimized Construct #3 | 0.10 | n/a |
| MC1 | 23.59 | 236 |
| MC1 MMP9-activated | 0.14 | n/a |
| MC2 | 28.25 | 283 |
| MC2 MMP9-activated | 0.31 | n/a |
| MC3 | 11.53 | 115 |
| MC3 MMP9-activated | 0.18 | n/a |
| MC4 | 10.94 | 109 |
| MC4 MMP9-activated | 0.12 | n/a |

### Mixed Lymphocyte Reaction (MLR) Assay

All test compounds induced the secretion of IL-2 with a dose-dependent effect (FIG. 33A). A similar observation was made for IFN-γ.

After MMP9-mediated cleavage of their masking moiety, all four bispecific binding proteins were more active than their masked counterparts MC1-MC4, with an EC₅₀ similar to “Optimized Construct #3” (unmasked) (Table 24). Overall, we observed a donor- and cytokine-dependent 1- to 2-log masking shift (IL-2: FIG. 33B; IFN-γ: FIG. 33C).

TABLE 24
MLR assay - EC₅₀ (nM) - median of 6 donors

| Test articles | IFN-γ | IL-2 |
|---|---|---|
| Optimized Construct #3 | 28.62 | 0.96 |
| MC1 | 90.98 | 8.27 |
| MC1 MMP9-activated | 8.10 | 1.24 |
| MC2 | 8.06 | 39.94 |
| MC2 MMP9-activated | 9.01 | 1.37 |
| MC3 | 385.66 | 2.72 |
| MC3 MMP9-activated | 10.16 | 0.98 |
| MC4 | 130.86 | 3.31 |
| MC4 MMP9-activated | 29.72 | 1.17 |

### CD3-PBMC Activation Assay

All test compounds induced the secretion of IL-2 with a dose-dependent effect (FIG. 34A). A similar observation was made for IFN-γ, TNF-α, and IL-5.

The conditionally-active anti-4-1BB/anti-PD-1 bispecific binding proteins with cleavable linker #1 (MC1 and MC2) had an EC₅₀ higher than those with cleavable linker #2 (MC3 and MC4), although in an equivalent nM range (Table 25).

After MMP9-mediated cleavage of their masking moiety, all four bispecific binding proteins were more active than their masked counterparts MC1-MC4, with an EC₅₀ similar to “Optimized Construct #3” (unmasked) (Table 25). Overall, we observed a donor- and cytokine-dependent 1- to 2-log masking shift (IL-2: FIG. 34B; IFN-γ: FIG. 34C; TNF-α: FIG. 34D; IL-5: FIG. 34E).

TABLE 25
CD3-PBMC activation assay - EC₅₀ (nM) - median of 9 donors

| Test articles | IFN-γ | TNF-α | IL-5 | IL-2 |
|---|---|---|---|---|
| Optimized Construct #3 | 1.12 | 0.97 | 1.54 | 3.46 |
| MC1 | 6.86 | 4.98 | 7.50 | 30.28 |
| MC1 MMP9-activated | 1.75 | 1.72 | 2.27 | 3.95 |
| MC2 | 3.85 | 5.03 | 8.95 | 50.20 |
| MC2 MMP9-activated | 1.92 | 1.81 | 2.47 | 5.53 |
| MC3 | 3.53 | 2.29 | 3.78 | 10.29 |
| MC3 MMP9-activated | 2.62 | 1.60 | 3.35 | 5.81 |
| MC4 | 3.43 | 2.41 | 4.96 | 25.83 |
| MC4 MMP9-activated | 1.59 | 1.97 | 3.12 | 5.17 |

### Plasma Stability

As shown in Table 26, all four masked compounds MC1-MC4 have a very good stability in mouse, cynomolgus and human plasma over time. MC3 and MC4 masked compounds are slightly more sensitive to cleavage than MC1 and MC2 as they comprise a uPA cleavage site in addition to the MMP cleavage site.

TABLE 26
stability in mouse, cynomolgus and human plasma

| | Percentage (%) of cleaved compound | | |
|---|---|---|---|
| | after 168 hours incubation in plasma | | |
| Test article | Mouse plasma | Cynomolgus plasma | Human plasma |
| MC1 | 0.34 | 0.15 | <1 |
| MC2 | 0.23 | 0.17 | <1 |
| MC3 | 3.0 | 8.1 | 2.6 |
| MC4 | 1.7 | 4.9 | 4.2 |

### In Vivo Efficacy Study

A robust anti-tumor activity of “Optimized Construct #3” (unmasked) was observed at low, medium and high doses; test compound MC1 showed equivalent activity versus the unmasked “Optimized Construct #3”, at the same doses (Table 27; FIG. 35).

TABLE 27
efficacy study on hPD-1/h4-1BB dKI mice bearing MC38 tumors

| | | | | Regressions | |
|---|---|---|---|---|---|
| Test articles | | DPI | ΔT/ΔC % | PR | CR |
| Isotype control | High dose | 21 | 100 | 0/11 | 0/11 |
| Optimized | Low dose | 21 | <0 | 6/11 | 6/11 |
| Construct #3 | Medium dose | 21 | <0 | 10/11 | 9/11 |
| | High dose | 21 | <0 | 8/11 | 8/11 |
| MC1 | Low dose | 21 | <0 | 7/11 | 6/11 |
| | Medium dose | 21 | <0 | 10/11 | 9/11 |
| | High dose | 21 | <0 | 10/11 | 10/11 |

DPI: days post-implant; PR/partial regression; CR: complete regression; ΔT/ΔC %: percentage ratio of the median tumor volume for the treated vs control group.

In conclusion, the masking moiety did not impact the anti-tumor efficacy of MC1 in vivo.

### Sequences

Masking moiety #1 (MM1) has an amino acid sequence set forth as:

(SEQ ID NO: 44)
EVGSLFANVEVCPELQGIFCYR

Masking moiety #2 (MM2) has an amino acid sequence set forth as:

(SEQ ID NO: 45)
EVGSSVLDVEVCPELQGIFCYR

Cleavable linker #1 has an amino acid sequence set forth as:

(SEQ ID NO: 46)
GGGPLGLAGSGGS

wherein PLGLAG (SEQ ID NO: 56, in bold) is a matrix metalloproteinase-9 (MMP-9) substrate.

Cleavable linker #2 has an amino acid sequence set forth as:

(SEQ ID NO: 47)
SGRSAGGGGPLGLAGSGGS

wherein SGRSA (SEQ ID NO: 57, underlined) is a urokinase-type plasminogen activator (uPa) substrate, and PLGLAG (SEQ ID NO: 56, in bold) is a matrix metalloproteinase-9 (MMP-9) substrate.

The amino acid sequence of the masked anti-4-1BB clone #2.1 V_{HH} is as set forth in any of SEQ ID NO: 48 to 51.

(SEQ ID NO: 48)
EVGSLFANVEVCPELQGIFCYRGGGPLGLAGSGGSEVQLVESGGGVVQPG
GSLRLSCAASGGLFSINTGGWYRQAPGKQRELVATITHDDRTNYAESVKG
RFTISRDNAKNTVYLQMNSLRPEDTALYYCRLGSAAIRGYWGQGTLVTVS
S
(SEQ ID NO: 49)
EVGSSVLDVEVCPELQGIFCYRGGGPLGLAGSGGSEVQLVESGGGVVQPG
GSLRLSCAASGGLFSINTGGWYRQAPGKQRELVATITHDDRTNYAESVKG
RFTISRDNAKNTVYLQMNSLRPEDTALYYCRLGSAAIRGYWGQGTLVTVS
S
(SEQ ID NO: 50)
EVGSLFANVEVCPELQGIFCYRSGRSAGGGGPLGLAGSGGSEVQLVESGG
GVVQPGGSLRLSCAASGGLFSINTGGWYRQAPGKQRELVATITHDDRTNY
AESVKGRFTISRDNAKNTVYLQMNSLRPEDTALYYCRLGSAAIRGYWGQG
TLVTVSS
(SEQ ID NO: 51)
EVGSSVLDVEVCPELQGIFCYRSGRSAGGGGPLGLAGSGGSEVQLVESGG
GVVQPGGSLRLSCAASGGLFSINTGGWYRQAPGKQRELVATITHDDRTNY
AESVKGRFTISRDNAKNTVYLQMNSLRPEDTALYYCRLGSAAIRGYWGQG
TLVTVSS

All four conditionally-active candidates MC1-MC4 comprise the same light chain (LC) sequence set forth as SEQ ID NO: 11.

The heavy chain (HC) sequence of conditionally-active candidate MC1 is set forth as:

(SEQ ID NO: 52)
EVGSLFANVEVCPELQGIFCYRGGGPLGLAGSGGSEVQLVESGGGVVQPG
GSLRLSCAASGGLFSINTGGWYRQAPGKQRELVATITHDDRTNYAESVKG
RFTISRDNAKNTVYLQMNSLRPEDTALYYCRLGSAAIRGYWGQGTLVTVS
SGGGGSGGGGSGGGGSEVQLVESGGGVVQPGGSLRLSCAASGFTFSDHTM
TWVRQAPGKGLEWVSSISSGGSRIIYADSVKGRFTISRDNAKNTLYLQMN
SLRPEDTALYYCARGTRYKLSTSGQGTLVTVSSGGGGSDKTHTCPPCPAP
EAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEAL
HNHYTQKSLSLSPGGGGGSGGGGSGGGGSGGGGSGGGGSQLQLQESGPGL
VKPSETLSLTCTVSGGSISSSSYFWGWIRQPPGKGLEWIGSIYRSGSTYY
NPSLKSRVTISVDTSKNQFSLKLSSVTAADTAVYYCARGITGDPGDYWGQ
GTLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNT
KVDKKVEPKSC

The heavy chain (HC) sequence of conditionally-active candidate MC2 is set forth as:

(SEQ ID NO: 53)
EVGSSVLDVEVCPELQGIFCYRGGGPLGLAGSGGSEVQLVESGGGVVQPG
GSLRLSCAASGGLFSINTGGWYRQAPGKQRELVATITHDDRTNYAESVKG
RFTISRDNAKNTVYLQMNSLRPEDTALYYCRLGSAAIRGYWGQGTLVTVS
SGGGGSGGGGSGGGGSEVQLVESGGGVVQPGGSLRLSCAASGFTFSDHTM
TWVRQAPGKGLEWVSSISSGGSRIIYADSVKGRFTISRDNAKNTLYLQMN
SLRPEDTALYYCARGTRYKLSTSGQGTLVTVSSGGGGSDKTHTCPPCPAP
EAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEAL
HNHYTQKSLSLSPGGGGGSGGGGSGGGGSGGGGSGGGGSQLQLQESGPGL
VKPSETLSLTCTVSGGSISSSSYFWGWIRQPPGKGLEWIGSIYRSGSTYY
NPSLKSRVTISVDTSKNQFSLKLSSVTAADTAVYYCARGITGDPGDYWGQ
GTLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNT
KVDKKVEPKSC

The heavy chain (HC) sequence of conditionally-active candidate MC3 is set forth as:

(SEQ ID NO: 54)
EVGSLFANVEVCPELQGIFCYRSGRSAGGGGPLGLAGSGGSEVQLVESGG
GVVQPGGSLRLSCAASGGLFSINTGGWYRQAPGKQRELVATITHDDRTNY
AESVKGRFTISRDNAKNTVYLQMNSLRPEDTALYYCRLGSAAIRGYWGQG
TLVTVSSGGGGSGGGGSGGGGSEVQLVESGGGVVQPGGSLRLSCAASGFT
FSDHTMTWVRQAPGKGLEWVSSISSGGSRIIYADSVKGRFTISRDNAKNT
LYLQMNSLRPEDTALYYCARGTRYKLSTSGQGTLVTVSSGGGGSDKTHTC
PPCPAPEAAGGPSVFLFPPKPKDTLMISRTPEVTCVVVDVSHEDPEVKFN
WYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCS
VMHEALHNHYTQKSLSLSPGGGGGSGGGGSGGGGSGGGGSGGGGSQLQLQ
ESGPGLVKPSETLSLTCTVSGGSISSSSYFWGWIRQPPGKGLEWIGSIYR
SGSTYYNPSLKSRVTISVDTSKNQFSLKLSSVTAADTAVYYCARGITGDP
GDYWGQGTLVTVSSASTKGPSVFPLAPSSKSTSGGTAALGCLVKDYFPEP
VTVSWNSGALTSGVHTFPAVLQSSGLYSLSSVVTVPSSSLGTQTYICNVN
HKPSNTKVDKKVEPKSC

The heavy chain (HC) sequence of conditionally-active candidate MC4 is set forth as:

(SEQ ID NO: 55)
EVGSSVLDVEVCPELQGIFCYRSGRSAGGGGPLGLAGSGGSEVQLVESGG
GVVQPGGSLRLSCAASGGLFSINTGGWYRQAPGKQRELVATITHDDRTNY
AESVKGRFTISRDNAKNTVYLQMNSLRPEDTALYYCRLGSAAIRGYWGQG
TLVTVSSGGGGSGGGGSGGGGSEVQLVESGGGVVQPGGSLRLSCAASGFT
FSDHTMTWVRQAPGKGLEWVSSISSGGSRIIYADSVKGRFTISRDNAKNT
LYLQMNSLRPEDTALYYCARGTRYKLSTSGQGTLVTVSSGGGGSDKTHTC
PPCPAPEAAGGPSVFLFPPKPKDTLMISRTPEVTCVVVDVSHEDPEVKFN
WYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCS
VMHEALHNHYTQKSLSLSPGGGGGSGGGGSGGGGSGGGGSGGGGSQLQLQ
ESGPGLVKPSETLSLTCTVSGGSISSSSYFWGWIRQPPGKGLEWIGSIYR
SGSTYYNPSLKSRVTISVDTSKNQFSLKLSSVTAADTAVYYCARGITGDP
GDYWGQGTLVTVSSASTKGPSVFPLAPSSKSTSGGTAALGCLVKDYFPEP
VTVSWNSGALTSGVHTFPAVLQSSGLYSLSSVVTVPSSSLGTQTYICNVN
HKPSNTKVDKKVEPKSC

1. A multispecific antigen-binding protein comprising at least one immunoglobulin single variable domain (ISV) specifically binding to 4-1BB, wherein the at least one ISV specifically binding to 4-1BB has pure agonist activity.

2. The multispecific antigen-binding protein according to claim 1, wherein pure agonist activity means that the ISV is capable of activating a T-cell via 4-1BB signaling (i) in soluble conditions, and/or (ii) in the absence of a cross-linking reagent, and/or (iii) in an FcγR-independent manner, and/or (iv) in the absence of target-mediated crosslinking of 4-1BB;
optionally wherein pure agonist activity is determined by an NF-κB pathway activation assay in the absence of a cross-linking reagent.

3. The multispecific antigen-binding protein according to claim 1, wherein the at least one ISV specifically binding to 4-1BB competes with 4-1BBL for 4-1BB binding.

4. The multispecific antigen-binding protein according to claim 1, wherein the at least one ISV specifically binding to 4-1BB interacts with the cysteine-rich domain 2 (CRD2) and/or cysteine-rich domain 3 (CRD3) domain of 4-1BB;
preferably the at least one ISV specifically binding to 4-1BB interacts with the CRD2 and CRD3 domains of 4-1BB.

5. The multispecific antigen-binding protein according to claim 1, wherein the at least one ISV specifically binding to 4-1BB interacts with one or several amino acid residues of 4-1BB selected from the group consisting of residues K69, G70, V71, F72, R73, F92, L95, S100, M101, C102, E103, Q104, K114, K115 and G116 of SEQ ID NO: 13.

6. The multispecific antigen-binding protein according to claim 1, wherein the at least one ISV specifically binding to 4-1BB comprises three complementary determining regions CDR1, CDR2 and CDR3; and
wherein CDR3 comprises or consists of the amino acid sequence ARTERIALS' (SEQ ID NO: 14), ARGTRYKMST (SEQ ID NO: 15), or ARGTRYKIFA (SEQ ID NO: 62).

7. The multispecific antigen-binding protein according to claim 6, wherein CDR1 comprises or consists of the amino acid sequence GFTFSDHT (SEQ ID NO: 16), GFAFRDFT (SEQ ID NO: 66), GDTFSSYA (SEQ ID NO: 67), or GFTFANYR (SEQ ID NO: 68).

8. The multispecific antigen-binding protein according to claim 6, wherein CDR2 comprises or consists of the amino acid sequence ISSGGSRI (SEQ ID NO: 17), INPSGGSQ (SEQ ID NO: 77), or IKKSGNRT (SEQ ID NO: 78).

9. The multispecific antigen-binding protein according to claim 1, wherein the at least one ISV specifically binding to 4-1BB comprises or consists of:
(i) an amino acid sequence selected from the group consisting of SEQ ID NOs: 2, 3, 58, 59, 60 and 61; or
(ii) an amino acid sequence sharing at least 70% of sequence identity over the non-CDR regions of SEQ ID NOs: 2, 3, 58, 59, 60 or 61.

10. The multispecific antigen-binding protein according to claim 1, wherein the at least one ISV specifically binding to 4-1BB comprises or consists of an amino acid sequence of SEQ ID NO: 2 or 3;
preferably wherein the at least one ISV specifically binding to 4-1BB comprises or consists of an amino acid sequence of SEQ ID NO: 3.

11.-15. (canceled)

16. The multispecific antigen-binding protein according to claim 1, comprising at least two ISVs, wherein one of the at least two ISVs specifically binds to 4-1BB, and one of the at least two ISVs specifically binds to another target antigen.

17. The multispecific antigen-binding protein according to claim 16, wherein the other target antigen is a T-cell antigen, a tumor-associated or tumor-specific antigen, or a non-self antigen.

18.-22. (canceled)

23. The multispecific antigen-binding protein according to claim 1, further comprising at least one Fab fragment.

24. The multispecific antigen-binding protein according to claim 1, wherein the multispecific antigen-binding protein comprises:
a) a first polypeptide comprising, preferably from N-terminus to C-terminus:
i. a first ISV specifically binding to 4-1BB;
ii. a second ISV specifically binding to 4-1BB, preferably wherein the second ISV is different from the first ISV;
iii. at least one C_{H} domain of a Fc region; and
iv. a variable and constant domain of a Fab fragment;
b) a second polypeptide comprising a variable and constant domain of a Fab fragment; wherein the variable and constant domains of the first and second polypeptide form a Fab fragment.

25.-37. (canceled)

38. The multispecific antigen-binding protein according to claim 23, wherein the at least one Fab fragment is an antigen-binding protein that specifically binds to PD-1, and comprises a light chain variable region with SEQ ID NO: 7 and a heavy chain variable region with SEQ ID NO: 8.

39.-41. (canceled)

42. A conditionally-active multispecific antigen-binding protein, comprising:
(i) a multispecific antigen-binding protein according to claim 1, and
(ii) at least one masking moiety reducing or inhibiting the binding of the multispecific antigen-binding protein to at least one of its target antigens.

43.-53. (canceled)

54. An immunoglobulin single variable domain (ISV) specifically binding to 4-1BB, wherein the ISV has pure agonist activity.

55.-66. (canceled)

67. An immunoglobulin single variable domain (ISV) specifically binding to 4-1BB, wherein the ISV comprises or consists of:
(i) an amino acid sequence selected from the group consisting of SEQ ID NOs: 1 and 4, or
(ii) an amino acid sequence sharing at least 70% sequence identity over the non-CDR regions of SEQ ID NOs: 1 or 4.

68.-72. (canceled)

73. A conditionally-active immunoglobulin single variable domain (ISV), comprising:
(i) an ISV according to claim 67, and
(ii) at least one masking moiety reducing or inhibiting the binding of the ISV to its target antigen.

74.-83. (canceled)

84. A conditionally-active immunoglobulin single variable domain (ISV) specifically binding to 4-1BB, comprising:
a. an ISV specifically binding to 4-1BB; and
b. at least one masking moiety reducing or inhibiting the binding of the ISV to 4-1BB.

85.-94. (canceled)

95. A conditionally-active immunoglobulin single variable domain (ISV) specifically binding to a target antigen, comprising:
c. an ISV specifically binding to a target antigen; and
d. at least one masking moiety reducing or inhibiting the binding of the ISV to its target antigen.

96.-105. (canceled)

106. An antibody or antigen-binding fragment thereof comprising:
(i) three light chain complementarity determining region (CDR) sequences found in SEQ ID NO: 7 or 5, and
(ii) three heavy chain CDR sequences found in SEQ ID NO: 8 or 6.

107.-112. (canceled)

113. A composition comprising the multispecific antigen-binding protein according to claim 1, and a pharmaceutically acceptable carrier or excipient.

114.-121. (canceled)

122. A method of treating a subject in need thereof, comprising administering an effective amount of the composition according to claim 113 to the subject.

123.-128. (canceled)

129. An isolated polynucleotide encoding the multispecific antigen-binding protein according to claim 1.

130.-137. (canceled)

138. A vector comprising the polynucleotide according to claim 129.

139. A host cell comprising the polynucleotide according to claim 129.

140. A method of making the multispecific antigen-binding protein comprising expressing the polynucleotide of claim 129 in a cell.

141.-148. (canceled)
